(19) United States
(12) Patent Application Publication
(10) Pub. No.: US 2026/0337688 A1
(43) Pub. Date: Oct. 29, 2026

(54) METHODS AND SYSTEMS FOR DIAGNOSING A PLURALITY OF MOTOR NEURON DISEASES AND/OR NEURODEGENERATIVE DISORDERS

(71) Applicant: BRAIN CHEMISTRY LABS, Jackson, WY (US)

(72) Inventors: Paul Alan COX, Jackson, WY (US); Sandra Anne BANACK, Jackson, WY (US); Rachael DUNLOP, Jackson, WY (US)

(21) Appl. No.: 19/651,198

(22) Filed: Apr. 17, 2026

Related U.S. Application Data
(60) Provisional application No. 63/791,303, filed on Apr. 18, 2025.
(60) Provisional application No. 63/912,489, filed on Nov. 6, 2025.

(51) Int. Cl. C12Q 1/6883; C12Q 1/6809

(52) U.S. Cl. CPC C12Q 1/6883; C12Q 1/6809; C12Q 2600/106; C12Q 2600/178

(57) ABSTRACT

Presented herein are methods, systems, and a model useful in identifying a subject who has, or is at risk of developing a motor neuron disease and/or neurodegenerative disorder. Certain embodiments also relate to development and use of a motor neuron disease and/or neurodegenerative disorder miRNA panel containing specific miRNAs, or subsets thereof, that are predictive and/or diagnostic of any motor neuron disease and/or neurodegenerative disorder, or that are predictive and/or diagnostic of one or more motor neuron diseases and/or neurodegenerative disorders selected from at least amyotrophic lateral sclerosis (ALS), primary lateral sclerosis (PLS), Alzheimer's Disease (AD), Mild Cognitive Impairment (MCI), Parkinson's Disease (PD), Multiple Sclerosis (MS), Huntington's Disease (HD), prion diseases such as Creutzfeld-Jakob disease (CJD), Lewy Body Disease (LBD), Multiple System Atrophy (MSA), Frontotemporal Dementia (FTD), Progressive Supranuclear Palsy (PSP), and controls without neurological disease as well as methods of monitoring treatments, methods of treatment, and kits for diagnostic purposes.

### FIELD OF THE EMBODIMENTS

This application claims priority to U.S. Provisional Application Ser. No. 63/791,303, filed on Apr. 18, 2025, and U.S. Provisional Application Ser. No. 63/912,489, filed on Nov. 6, 2025, the disclosures of each of which are incorporated herein in their entireties.

Certain embodiments relate to methods of developing a multi-disorder detection method and system/model, and to methods of detecting the presence of, the absence of, or the amount of one or more miRNAs in bodily fluids, including circulating blood, and determining the presence or absence of one or more neurological disorders using the techniques described herein. Certain embodiments also relate to development and use of a motor neuron disease and/or neurodegenerative disorder miRNA panel containing specific miRNAs, or subsets thereof, that are predictive, prognostic, and/or diagnostic of having a neurodegenerative disease and/or neurodegenerative disorders in general, or that are predictive, prognostic, and/or diagnostic of one or more specific motor neuron diseases and/or neurodegenerative disorders selected from at least amyotrophic lateral sclerosis (ALS), primary lateral sclerosis (PLS), Alzheimer's Disease (AD), Mild Cognitive Impairment (MCI), Parkinson's Disease (PD), Multiple Sclerosis (MS), Huntington's Disease (HD), prion diseases such as Creutzfeld-Jakob disease (CJD), Progressive Supranuclear Palsy (PSP), Lewy body disease (LBD), Multiple System Atrophy (MSA), Frontotemporal Dementia (FTD), and controls without neurological disease as well as methods of monitoring treatments, methods of treatment, and kits for diagnostic purposes. Other embodiments include a model useful in determining the presence or absence of one or more of the above motor neuron diseases and/or neurodegenerative disorders that includes a database of miRNA, coefficients assigned to each miRNA, and software configured to analyze miRNA data from a sample to predict the presence or absence of the one or more of the above motor neuron diseases and/or neurodegenerative disorders.

Other embodiments include a method for determining which miRNA are useful in diagnosing, predicting a disease outcome, or measuring severity of disease.

### INTRODUCTION

The need for biomarkers for all neurodegenerative diseases is well established. See, ALS Strategic Plan 2023, available on nih.gov; Vignaroli, et al., “The Need for Biomarkers in the ALS-FTD Spectrum: A Clinical Point of View on the Role of Proteomics,” Proteomes 11(1):1-18 (2023). Biomarkers are essential for reducing diagnostic delays and improving disease outcomes as well as for therapeutic drug development. To improve reproducibility and ultimately to achieve biomarker adoption, every biomarker should undergo robust validation (U.S. Department of Health and Human Services. Biomarker Qualification: Evidentiary Framework Guidance for Industry and Staff 2018, available at fda.gov). The most effective biomarkers are reliable measures of disease (or disease state), applicable across drug development trials, and independent of the drug being tested.

Applicant has previously filed an application for detecting and/or diagnosing ALS by relying on determining the presence of, or absence of, certain miRNA in bodily fluids that were either present in exosomes, or in circulating bodily fluids. U.S. Patent Application Publication No. 2021/0164051, and U.S. patent application Ser. No. 18/974,425, filed on Dec. 9, 2024, docket No. 042733-0579307, previously filed by applicant, are hereby incorporated by reference in their entireties. Applicant also has previously disclosed an application entitled: “Blood Indicators of Alzheimer's Disease,” U.S. Patent Application Publication No. 2022/0252620, the disclosure of which is incorporated by reference herein in its entirety. This prior application discloses methods of detecting the presence, absence or amount of 2-aminoethyl dihydrogen phosphate and/or taurine in a sample.

Amyotrophic lateral sclerosis (ALS) is a rare neurological disease with an estimated 30,000 active cases per year in the United States. Incident rates in 2018 were 1.6 per 100,000 USA population and prevalence was 9.1 per 100,000 population (Mehta P, et al., “Prevalence of amyotrophic lateral sclerosis in the United States,” Amyotroph Lateral Scler Frontotemporal Degener, August 30:1-7 (2023); doi: 10.1080/21678421.2023.22458584). The disease typically develops during mid-to-late life, progresses rapidly, and is terminal within two to five years but some patients survive much longer. See also, Shefner J M et al., “A proposal for new diagnostic criteria for ALS,” Clin. Neurophysiol, 113: pp 1975-1978 (2020); Brown R H, et al., “Amyotrophic lateral sclerosis,” N Engl J Med., 377(2): 162-172 (2017). The clinical presentation of ALS is also variable with distal muscle limb weakness more common than bulbar onset (Verma A., “Clinical Manifestation and Management of Amyotrophic Lateral Sclerosis in Amyotrophic Lateral Sclerosis, In: Toshiyuki A, ed. “Amyotrophic Lateral Sclerosis,” Exon Publication; 1-14.7 (2021). Familial ALS represents approximately 10% of all cases with the remaining 90% of sporadic cases occurring in individuals with no known genetic mutations (Masrori P, et al., “Amyotrophic lateral sclerosis: a clinical review,” Eur J Neurol., 27(10):1918-1929 (2020). Care for ALS patients remains supportive and palliative, while a cure, and even meaningfully effective therapy, remains elusive. See, Verma A, above.

An ALS diagnosis typically is made by a clinician after observation of progressive degeneration of upper and lower motor neurons using standardized clinical criteria. However, patients frequently experience diagnostic uncertainty resulting in delayed diagnosis, an increased number of physician consultations, and sometimes unnecessary medical procedures (Richards D, et al., “Time to diagnosis and factors affecting diagnostic delay in amyotrophic lateral sclerosis,” J Neurol Sci., 417:117054 (2020), doi: 10.1016/j.jns.2020.117054.9. Misdiagnosis can be as high as 68%, partly because family physicians and some general neurologists outside of large metropolitan areas do not observe many cases in their lifetimes. A robust ALS diagnostic biomarker would give patients a chance at earlier treatment and novel therapeutic intervention. It could also accelerate the testing of new drug candidates in clinical trials (Kiernan M C, et al., “Improving clinical trial outcomes in amyotrophic lateral sclerosis,” Nature Reviews Neurology, 17(2):104-118.10 (2021).

ALS biomarker research, both diagnostic (Gomes B C, et al., “Differential Expression of miRNAs in Amyotrophic Lateral Sclerosis Patients,” Mol Neurobiol., August 2:1-14 (2023)), and prognostic, (Magen I, et al., “Circulating miR-181 is a prognostic biomarker for amyotrophic lateral sclerosis,” Nature neuroscience, 24(11):1534-1541 (2021)) is on-going, with recent developments in neuroimaging, electrophysiology, and fluid-based markers. See Ilieva H, Vullaganti M, Kwan J., “Advances in molecular pathology, diagnosis, and treatment of amyotrophic lateral sclerosis,” BMJ, 383:e075037 (2023). Imaging biomarkers can be difficult for ALS patients as symptom-related complications make these procedures uncomfortable (Sturmey E, Malaspina A. “Blood biomarkers in ALS: Challenges, applications and novel frontiers,” Acta Neurologica Scandinavica, 146(4):375-388 (2022)). Blood or urine-based biomarkers are generally considered preferable to cerebrospinal fluid markers because they are less invasive. Two candidates, neurofilament and p75 neurotrophin receptor, are being investigated as pharmacological markers in conjunction with ALS clinical trials. See also, Shepheard S R, et al., “The extracellular domain of neurotrophin receptor p75 as a candidate biomarker for amyotrophic lateral sclerosis,” PLoS One, 9:e87398 (2014); Jourdi G, et al., “Soluble p75 neurotrophic receptor as a reliable biomarker in neurodegenerative diseases: what is the evidence?”, Neural Regen Res, 19(3):536-541 (2024). RNA biomarkers are also of interest as ALS biomarkers and in combination with neurofilament, may have promise in survival prognostication. See Joilin G, et al., “An Overview of MicroRNAs as Biomarkers of ALS,” Front Neurol, 10:186 (2019); Zhu Y, et al., “Evaluating the causal association between microRNAs and amyotrophic lateral sclerosis,” Neurol Sci., 44(10):3567-3575 (2023); Shen D, et al., “Single-Cell RNA Sequencing Analysis of Microglia Dissected the Energy Metabolism and Revealed Potential Biomarkers in Amyotrophic Lateral Sclerosis,” Mol Neurobiol. (2023); and Grima N, et al., “RNA sequencing of peripheral blood in amyotrophic lateral sclerosis reveals distinct molecular subtypes: Considerations for biomarker discovery,” Neuropathol Appl Neurobiol. 49(6):e12943 (2023). A recent study has demonstrated the potential of HDGFL2, a cryptic neoepitope, as a marker of presymptomatic ALS (Irwin K E, et al., “A fluid biomarker reveals loss of TDP-43 splicing repression in presymptomatic ALS-FTD,” Nat Med., 30(2):382-393 (2024); doi: 10.1038/s41591-023-02788-5. Erratum in: Nat Med. 2024 Apr. 5: PMID: 38278991; PMCID: PMC10878965.

While PLS and ALS have overlapping symptoms, PLS involves only the upper motor neurons (UMN) while ALS involves both UMN and lower motor neurons. PLS also progresses more slowly than ALS resulting in a longer life expectancy after diagnosis. It would be desirable to develop a blood-based diagnostic ALS and PLS biomarker, or biomarker set that could reliably identify patients who had previously been diagnosed using standardized clinical criteria. If such a diagnostic could be discovered, the inventors hypothesized that patterns and concentrations of certain miRNA within the circulating blood system might be useful with sensitivity and specificity to aid in ALS and/or PLS diagnosis, in ALS and/or PLS monitoring, and in assessing efficacies of therapies for ALS and/or PLS with greater speed and accuracy than previous methods that involved isolating exosomes.

Ninety-five percent of Alzheimer's disease (AD) cases occur in people over 60-65 years old. This is typically a time in life when individuals are looking forward to their golden years and are poised to make significant contributions to their families and communities. AD robs them not only of life, but prior to death burdens them with inability to form or access short-term memories, increasing confusion, disorientation, social withdrawal, and increasing dementia which intensifies during their last years of life. Neuropathologically, onset of AD symptoms are sometimes correlated with brain neuropathology, particularly amyloid-β-42 (Aβ42) plaques and a dense tauopathy of neurofibrillary tangles (NFTs) composed of hyperphosphorylated tau deposits in specific brain regions.

There is likely a significant latency period between the initiation of AD-type neuropathology in the brain and onset of clinical symptoms. A study using ¹⁴C decay from detonation of the 50 megaton Tsara bomba by the Soviet Union on Oct. 31, 1961, which injected a tremendous amount of ¹⁴C into the atmosphere, allowed precise dating of NFTs and Aβ42 plaques extracted from postmortem brain tissues from AD patients. In two of the six patients studied, the NFTs were dated seven years prior to onset of symptoms and in one patient the Aβ42 plaque dated nine years prior to symptom onset. The advantage of discovering and using biomarkers for AD, particularly during the latency period, could be used to explore new types of treatment or even test previously failed drugs, which could eventually be prescribed at a much earlier period of disease progression.

A recent meta-analysis suggested that several CSF biomarkers resulting from amyloid deposition, neuronal damage and loss, and the formation of NFTs, notably phosphorylated-tau (P-tau), amyloid-beta (Aβ42), total-tau (T-tau), as well as neurofilament light protein (NFL), were strongly associated with AD. Blood plasma concentration of T-tau was also indicated as a potentially good indicator of AD, requiring further research, while plasma concentrations of Aβ40 and Aβ42 did not appear to be good candidates for AD diagnosis. Imaging biomarkers assessing amyloid-beta plaques (PiB-PET scans), tau deposits (tau-PET), brain atrophy (structural MRI), memory-related activity patterns (fMRI), and decreased glucose metabolism (FDG-PET) are also currently in use as AD biomarkers.

Current diagnosis of AD remains uncertain (Dubois et al Lancet Neurol. 2014; 13(6):614-29) and it is based on combination of battery of clinical and neuropsychological tests and neuroimaging, such as structural imaging using Magnetic Resonance Imaging (MRI) and/or glucose metabolism using Positron emission tomography (PET) of fluorodeoxyglucose F18 (¹⁸F-FDG), with an accuracy of less than 70%-85% depending on the method and the severity stage of the disease (Frisoni et al., Nat Rev Neurol. 2010; 6(2):67-77; Tahmasian et al., J Nucl Med. 2016; 57(3):410-5. More recently a plasma analyses by mass spectrometry is being used to determine the ratio of plasma phosphorylated tau 217 (p-tau217) to non-p-tau217 and a ratio of amyloid-β 42 and amyloid-β 40 (Palmqvist et al. 2024).

Some research has been conducted on the use of micro RNA (miRNA) and long non-coding RNA (lncRNA) as diagnostic biomarkers for AD because they are known to circulate and tissue specific profiles can be identified in body fluids such as serum, plasma, CSF, and urine. U.S. Pat. No. 11,718,879 claims a method of detecting levels of lncRNAs in a lncRNA signature that includes 13 lncRNAs of Inc-DLGS5-1:1, Inc-EBLN1-1:4, Inc-FAT1-7:2, Inc-PRR5-5:1, Inc-RBKS-6:1, Inc-FOXD4L5-35:1, Inc-TENM3-3:3, Inc-FAM133B-2:1, Inc-ZNF726-1:3, Inc-AP3M1-1:1, Inc-DUSP10-6:1, Inc-TPPP-1:2, and LINC01206:20. This patent discloses a number of additional signatures including miRNA signatures that include miR.99a.5p, and signatures of 3, 7, 11, 15, 28, and 74 miRNAs.

Others have disclosed measuring levels of or determining the presence or absence of miRNA in fluids to diagnose various neurodegenerative diseases. For example, U.S. Pat. No. 8,648,017 claims measuring levels of normalizer small RNA not expressed in the brain and selected from miR-181a, miR-491-5p, miR-10b, and miR-141. US Patent Application Publication No. 2023/0041359 discloses diagnosing a disease or disorder by measuring in a biological sample one or more miRNAs selected from over 47,972 miRNAs.

Parkinson's disease (PD) is the one of the most common neurodegenerative diseases, with a prevalence close to 1% in people over the age of 60. With the aging of the population, the incidence of PD patients is increasing. PD is a chronic progressive disease caused by the degeneration and severe loss of dopaminergic neurons in the in the substantia nigra pars compacta. Capriotti, et al., “Parkinson disease,” Home Health Now, Vol. 34, pp. 300-307 (2016). Its onset involves the combination of genetic and high-risk environmental factors. The most prominent clinical feature is motor dysfunction, including rigidity, resting tremor, bradykinesia, and postural instability. Tysnes, et al., “Epidemiology of Parkinson's disease,” J. Neur. Transm., Vol. 124 pp. 901-905 (2017). The clinical diagnosis of PD mainly depends on clinical manifestations and neurological examination. However, clinical symptoms and signs may vary from patient to patient and may overlap with other neurodegenerative diseases, so PD diagnosis remains a great challenge. The main reason for the difficulty of PD diagnosis is the complex pathogenesis, such as oxidative stress, neurotrophic factor deficiency, excitotoxicity, mitochondrial dysfunction, gene mutation, immune dysregulation and apoptosis. Yang, et al., “Circulating miRNAs and long non-coding RNAs as potential diagnostic biomarkers for Parkinson's disease,” Front Mol. Neurosci., Vol. 14:631553 (2021). Although imaging methods can aid in the clinical diagnosis of PD and the unified Parkinson's Hoehn-Yahr scale can help assess PD severity, but PD is highly heterogeneous, so there is still a lack of objective laboratory diagnostic methods.

Yang and others have proposed the use of circulating miRNAs as potential biomarkers for the diagnosis of PD. Yang, et al., “Circulating miRNAs and long non-coding RNAs as potential diagnostic biomarkers for Parkinson's disease,” Front Mol. Neurosci., Vol. 14:631553 (2021); Zhang, et al., “Circulating microRNAs as potential diagnostic biomarkers for the diagnosis of Parkinson's disease: A meta-analysis,” Neurologia, Vol. 39, pp. 573-583 (2024). These documents disclose a number of miRNA reported in the literature as allegedly useful as a biomarker for PD, either individually, or some in combination with one or more miRNA, and/or the use of miRNA clusters.

The first symptomatic stage of AD that is manifested by mild clinical symptoms is Mild Cognitive Impairment (MCI), which is usually defined as an intermediate state between normal aging and dementia (DeCarli, Lancet Neurol., 2003, 2:15-21; Stephan et al., Alzheimer's Res Therapy, 2009, 1:1-9; Apostolova et al., Human Brain Mapping, 2010, 31:786-797). On average, MCI patients convert to dementia at a rate of 10-15% annually (Petersen et al., Arch Neurol. 2001, 58:1985-1992; Apostolova et al., Human Brain Mapping, 2010, 31:786-797). However, currently the MCI outcome is not reliably predictable. First, up to 40% of MCI patients revert to normal status (Larrieu et al., Neurology, 2002, 59:1594-1599; Brooks, Loewenstein, Alzheimer's Res Therapy, 2010, 2:28-36), and autopsy studies demonstrate that a substantial percentage of MCI patients do not have evidence of AD pathology (Jicha et al., Arch Neurol, 2006, 63:674-681; Khan, Alkon, Neurobiol. Aging, 2010, 31:889-900). Second, about 20% of MCI patients who convert to dementia are diagnosed not with AD but other neurodegenerative diseases, such as vascular, Lewy body, Huntington, Parkinson, and other dementias (Jicha et al., Arch Neurol, 2006, 63:674-681; Stephan et al., Alzheimer's Res Therapy, 2009, 1:1-9). Third, disease progression varies for AD patients from slow to intermediate and rapid (Doody et al., Alzheimer's Res Therapy, 2010, 2:2-10). Even clinically MCI is not a homogeneous pathology and can be described as two conditions, with amnestic symptoms (aMCI) and without amnestic symptoms (Dlugaj et al., Dement Geriatr Cogn Disord., 2010, 30:362-373; Brooks, Loewenstein, Alzheimer's Res Therapy, 2010, 2:28-36). Some publications have demonstrated that aMCI converts to dementia much more often and is a better predictor of AD (Mariani et al., J Alzheimer's Dis., 2007, 12:23-35; Luck et al., Psychiatr Prax., 2008, 35:331-336; Koivunen et al., Neurology, 2011, 76:1085-1099). However, other authors have not found significant difference in the conversion rate for two MCI forms (Rountree et al., Dement Geriatr Cogn Disord., 2007, 24:476-482).

Exosomes have been shown to comprise nucleic acids, including messenger RNA (mRNA), microRNA (miRNA), and small interfering RNA (siRNA), which can be transferred from one cell to another. Exosomes that are released into the extracellular matrix and taken up by adjacent cells can potentiality transfer information from one cell to another. Such information can be therapeutic or pathogenic. Identifying, detecting and analyzing miRNAs associated with neural-derived exosomes (or neural-enriched extracellular vesicles (NEE), and use of such identification, detection, and analysis in diagnosing and early detection of motor neuron diseases, such as ALS, is described in U.S. Patent Application Publication No. 2021/0164051, the disclosure of which is incorporated herein by reference in its entirety.

As described therein, neural-derived exosome fractions, and/or fractions comprising portions or components of exosomes of neural cell or neural tissue origin (individually and collectively referred to interchangeably as “a neural-derived exosome fraction” or “neural-enriched exosome fractions” or “neural-enriched extracellular vesicles”), can be detected, isolated, enriched, or prepared from a sample, for example, a sample obtained from a subject. Thus, a sample may be obtained from a subject, neural derived exosomes isolated and enriched using the techniques described herein, and then the exosomes are analyzed for the presence, absence, and/or amount of miRNAs present in the neural-derived exosomes. Identifying the miRNA present in neural-derived exosomes by disease state is important for selecting the combination of miRNA useful to differentiate disease states and for selecting the combination of miRNA useful for prediction, diagnosis, and identifying disease progression. While these methods are useful in early diagnoses and early detection of motor neuron diseases, such as ALS, the method requires at least the isolation of neural derived exosomes from the sample. The use of neural-derived exosomes also is useful in determining correlations between one or more miRNA, typically 2 or 3, and the presence of indication of a neurodegenerative disorder, such that the presence, absence, and/or amount of a group of miRNAs selected using neural-derived exosomes can be used to diagnose, monitor, and assess treatment efficacy for certain neurodegenerative disorders. The inventors did not heretofore believe that analyzing circulating bodily fluids for the presence, absence, and/or amount of miRNAs without exosome isolation would or could provide an accurate detection or diagnosis of motor neuron diseases. It was not until a model was recently created by the inventors using exosome-derived miRNA that the inventors confirmed that detection of miRNA in circulating bodily fluids, such as blood or plasma, and then subjecting that analysis to the exosome-derived miRNA model was useful in diagnosing a variety of neurodegenerative disorders and/or motor neuron diseases.

While some of the afore-discussed subject matter is discussed with reference to known literature and publications, the embodiments described herein may include one or more known features, without suffering from drawbacks and/or problems previously encountered. In addition, information presented in this Introduction section is not an admission that the information is prior art to the embodiments described herein, expressly including the preceding paragraph.

### SUMMARY

Presented herein, in certain embodiments, are methods of developing a screening panel of miRNAs to detect and/or diagnose the presence of, or risk of developing a motor neuron disease or neurodegenerative disorder, or to detect and/or diagnose the presence of, or risk of developing one or more motor neuron diseases or neurodegenerative disorders, and identifying one or more motor neuron diseases or neurodegenerative disorders or combinations thereof. The one or more motor neuron diseases or neurodegenerative disorders may be selected from at least the following: amyotrophic lateral sclerosis (ALS), primary lateral sclerosis (PLS), Alzheimer's Disease (AD), Mild Cognitive Impairment (MCI), Parkinson's Disease (PD), Multiple Sclerosis (MS), Huntington's Disease (HD), prion diseases such as Creutzfeld-Jakob disease (CJD), Lewy body disease (LBD), Multiple System Atrophy (MSA), Frontotemporal Dementia (FTD), and Progressive Supranuclear Palsy (PSP). The method may comprise identifying a plurality of miRNA that are differentially expressed by comparing the amount of miRNA in certain test samples from subjects who were diagnosed as having at least one motor neuron disease or neurodegenerative disorder, to the amount of the same miRNA in a sample from a healthy subject, manipulating the expression levels by calculating the geometric mean (delta Cq) for each miRNA, determining the gene-fold expression for each miRNA from the delta Cq values, and/or assigning coefficients to the respective miRNA using one or more regression analyses, wherein the coefficients are indicative of each miRNA's relative contribution to the diagnosis of the particular motor neuron disease or neurodegenerative disorder.

In one embodiment, a model suitable for use in diagnosing the presence or absence of one or more motor neuron disease and/or neurodegenerative disorder is provided, along with a method of making the model. The model can be prepared by a method that includes generating a database comprising a screening panel comprising delta Cq values for sets of miRNA and coefficients corresponding to each miRNA in the respective sets, each set corresponding to a specific motor neuron disease and/or neurodegenerative disorder. The model may further be prepared by creating software configured to: access the database; process qPCR generated quantification data for each miRNA in each set of miRNA; generate normalized qPCR values for each miRNA in the list, and process the normalized qPCR values for each miRNA by computing and outputting a predicted diagnosis of the presence or absence of a motor neuron disease and/or neurodegenerative disorder using the software. The predictive certainty uses parameters pre-set into the software to provide the computation.

In accordance with the model and method, an miRNA screening panel can be created by isolating neural-enriched extracellular vesicles (NEEs) or exosomes from test samples from subjects who were diagnosed as having at least one motor neuron disease or neurodegenerative disorder, detecting and/or quantifying the amount of exosome-derived miRNAs for each disease or disorder that the respective subject was previously diagnosed with, comparing the presence and/or amount of exosome-derived miRNAs from the test samples from subjects who were diagnosed as having at least one motor neuron disease or neurodegenerative disorder with the same miRNAs from a sample of exosome-derived miRNAs from a healthy subject, and selecting those exosome-derived miRNAs for each disease or disorder whose gene-fold expressions (determined by normalizing the quantification cycle (Cq) values for each miRNA against three or more reference miRNA) are statistically significantly different from the gene-fold expressions of the same miRNA of healthy subjects, typically by being from about 1.1 to about 20 fold higher or lower than the gene-fold expressions of the respective miRNA of a healthy subject. In accordance with this method, a plurality of miRNAs will be separately identified for each of the motor neuron diseases and/or neurodegenerative disorders, such as but not limited to ALS, PLS, AD, MCI, PD, MS, HD, CJD, LBD, MSA, FTD, and PSP, thereby providing an exosome-derived miRNA screening panel that comprises a database of sets of miRNAs for each motor neuron diseases and/or neurodegenerative disorders.

The exosome-derived miRNA screening panel comprising the sets of miRNAs then can be used to create a database comprising the exosome-derived miRNA screening panel and coefficients corresponding to each miRNA in the panel. The coefficients associated with each miRNA in the database can be created by conducting logistic regression analyses on the initial miRNA panel (typically by carrying out logistic regression analyses for each set of miRNA for a specific motor neuron disease and/or neurodegenerative disorder) by subjecting the delta Cq values for each of the miRNA in the initial list to an iterative regression analysis using a Lasso selection feature by comparing delta Cq values of miRNA from known disease subjects (e.g., samples from subjects known to have AD, or MCI, or PD, as the case may be) to the delta Cq values of miRNA from known healthy controls. The iterative regression analysis with the Lasso selection feature can determine which miRNA contribute the most to the specific motor neuron disease and/or neurodegenerative disorder, and which miRNA contribute the most to healthy controls, and will generate a series of second, smaller lists of miRNA from the above initial lists present in the exosome-derived miRNA screening panel, along with a coefficient associated with each miRNA in the second list (each list of miRNA and coefficient corresponding to a specific motor neuron disease and/or neurodegenerative disorder). The coefficients represent the relative contribution of each miRNA in separating the specific motor neuron disease and/or neurodegenerative disorders from healthy control subjects.

The iterative regression analysis with the Lasso selection feature may end when the predictive accuracy of motor neuron disease and/or neurodegenerative disorders on the one hand, or healthy on the other hand, is at or above about 65% (or above about 70%, or above about 75%, or above about 80%, or above about 85%, or above about 90%), thereby generating a database that includes a series of second lists of miRNA and coefficients corresponding to each miRNA for each respective motor neuron disease and/or neurodegenerative disorder. The completion of the regression analysis also establishes the parameters in the software for carrying out subsequent regression analyses on samples from unknown subjects, thereby creating the software configured to process miRNA data from a sample from a subject and generate a diagnosis of whether the subject has, is susceptible to having, or does not have one or more motor neuron disease and/or neurodegenerative disorders selected from, for example, ALS, PLS, AD, MCI, PD, MS, HD, CJD, LBD, MSA, FTD, and PSP. The exosome-derived miRNA model can be continuously updated by repeating the above method with additional samples from subjects known to have one of the above motor neuron diseases and/or neurodegenerative disorders, or from healthy controls, where the updated model may create minor modifications to the miRNA in the second list along with their respective coefficients, and may modify the parameters in the software

Another embodiment includes a non-exosome-derived miRNA model, methods of making the model, and methods of using the model to diagnose the presence or absence of one or more motor neuron diseases and/or neurodegenerative disorders. A method of developing a non-exosome-derived miRNA model (including the screening panel, and subsequent database and software) may include carrying out the same method above, except the miRNA are isolated and detected from a test sample of a subject's blood, the subject having been previously diagnosed directly, without isolation of NEEs or exosomes, comparing the amounts or presence of each miRNA with control or healthy subject's miRNA, manipulating the miRNA as discussed above (by calculating Cq values, geometric means of the Cq values, determining gene-fold expression levels and differences between healthy and controls, subjecting the gene-fold expression to logistic regression analysis and assigning coefficients to each miRNA). In accordance with this method, a plurality of miRNAs will be separately identified for each of ALS, PLS, AD, MCI, PD, MS, HD, CJD, LBD, MSA, FTD, and PSP, thereby providing a non-exosome-derived or circulating miRNA model. In an embodiment, the miRNAs are initially identified using NEEs or exosomes and next generation sequencing. Once the miRNAs are identified, the diagnostic/prognostic test will be conducted directly using circulating blood.

In an embodiment, a method may also include comparing the plurality of miRNAs in the exosome-derived miRNA model with the plurality of miRNAs in the non-exosome-derived or circulating miRNA model, and selecting a plurality of miRNA for any neurodegenerative disease and/or neurodegenerative disorder in general, or selecting a plurality of miRNA for each of ALS, PLS, AD, MCI, PD, MS, HD, CJD, LBD, MSA, FTD, and PSP that are similarly detected and differentially expressed in each model. Similarly detected and differentially expressed denotes the respective miRNA have gene-fold expression amounts of from about 1.1 to about greater than 20-fold higher or lower than the control, and the respective miRNA are both either higher or lower. The selection of the plurality of miRNA for any neurodegenerative disease and/or neurodegenerative disorder in general, or selection a plurality of miRNA for each of ALS, PLS, AD, MCI, PD, MS, HD, CJD, LBD, MSA, FTD, and PSP provides a final miRNA model that can be used in the methods described further hereinbelow. Using the guidelines provided herein, the final miRNA model can be periodically updated to include additional motor neuron diseases, and/or neurodegenerative disorders.

Some embodiments include detection and analysis of miRNAs in circulating bodily fluids without exosome isolation in methods of detection and diagnosis of a motor neuron disease and/or neurodegenerative disorder, or in methods of detection and diagnosis of one or more of identified motor neuron diseases, and/or neurodegenerative disorders such as ALS, PLS, AD, MCI, PD, MS, HD, CJD, LBD, MSA, FTD, and PSP, and others. In some aspects, presented herein is method of identifying a subject who has, is at risk of developing a motor neuron disease and/or neurodegenerative disorder, or is being tested to exclude a possible future motor neuron disease and/or neurodegenerative disorder diagnosis comprising: (a) determining a presence or amount of one or more micro RNAs (miRNAs) in a sample obtained from the subject without determining the presence or amount in neural derived exosomes, wherein the one or more miRNA are selected from the group consisting of at least two, three, four, five, or more miRNAs in a final miRNA panel that contains a plurality of miRNAs for any motor neuron disease and/or neurodegenerative disorder in general, or a plurality of miRNAs for each of at least ALS, PLS, AD, MCI, PD, MS, HD, CJD, LBD, MSA, FTD, and PSP, and (b) determining if the subject has, or is at risk of developing any motor neuron disease and/or neurodegenerative disorder, or determining if the subject has, or is at risk of developing an identified motor neuron disease and/or neurodegenerative disorder according to the presence or amount of the one or more miRNAs in the sample.

In accordance with this method, the embodiments include using the model to diagnose a subject as either having or being susceptible of developing motor neuron disease and/or neurodegenerative disorder, or as not having or being susceptible of developing a motor neuron disease and/or neurodegenerative disorder. The method includes obtaining a sample from a subject, extracting miRNA from the sample, synthesizing cDNA for each miRNA listed in the model (e.g., those miRNA from the second list), amplifying the miRNA using, for example, qPCR and determining the quantification cycle (Cq) values for each miRNA. The raw cycle quantitation (Cq) numbers for the miRNA developed in the model can then be normalized by the geomean of three or more endogenous miRNAs, thus generating a delta Cq value for each miRNA, using the techniques discussed above. These delta Cq values are then input into the software created in developing the model in which they are subjected to a mathematical comparison to the logistic regression analysis with its predetermined parameters generated in developing the model.

The system may then determine how each miRNA value contributes to the prediction of disease state incrementally and will generate a coefficient for each miRNA that is indicative of the log-odds of the subject having or being susceptible of developing, or as not having or being susceptible of developing a motor neuron disease and/or neurodegenerative disorder. The method may further include combining the log-odds for each miRNA in the model to generate the final prediction that a person does or does not have, or is not susceptible to having a motor neuron disease and/or neurodegenerative disorder using a linear regression model used in predictive modeling. For example, the software may add up each of the log odds ratios using the coefficients assigned to each miRNA in the model, and then generate a prediction number that indicates that the person is predicted to have the disease with a certain percentage certainty, (e.g., person has ALS, or AD) with 95% certainty.

In some aspects, presented herein is a method of preventing or treating a motor neuron disease and/or neurodegenerative disorder in a subject who has, or is at risk of developing the motor neuron disease and/or neurodegenerative disorder, the method comprising: (a) determining if the subject has, or is at risk of developing the motor neuron disease and/or neurodegenerative disorder using the method described above; and (b) administering a motor neuron disease and/or neurodegenerative disorder treatment to the subject when the determining of (a) determines that the subject has, or is at risk of developing the motor neuron disease and/or neurodegenerative disorder.

In some aspects, presented herein is one or more kits useful in carrying out a method of identifying a subject who has, or is at risk of developing any motor neuron disease and/or neurodegenerative disorder, or identifying a subject who has, or is at risk of developing an identified motor neuron disease and/or neurodegenerative disorder that includes one or more multi-well plates, with each well containing one or more miRNA primers selected from the group consisting of at least two, three, four, five, or more miRNAs in a final miRNA panel that contains a plurality of miRNAs, and/or a final miRNA panel that contains a plurality of miRNAs for each of at least ALS, PLS, AD, MCI, PD, MS, HD, CJD, LBD, MSA, FTD, and PSP, and a mechanism to calculate the likelihood of a positive or negative diagnosis, and/or probability of having or not having a motor neuron disease and/or neurodegenerative disorder. In one embodiment, the kit includes two well-plates in which one or both well plates contain one or more miRNA primers selected from the list, and Quality Control (QC)/spike-in/calibration controls in which one or more wells contain QC primers, no-template controls, and/or spike-in primers designed to monitor the success of the method. In another embodiment, the kit includes one well-plate in which the wells include one or more miRNA primers selected from the list above, QC primers, no-template controls, and/or spike-in primers designed to monitor the success of the method. In a further embodiment, the mechanism to calculate the likelihood of a positive or negative diagnosis, and/or probability of having or not having a motor neuron disease and/or neurodegenerative disorder includes software configured to carry out the calculation using random forest machine learning algorithms, logistic regression algorithms, and/or other algorithms developed for classification of known disease and healthy control plasma samples.

Certain aspects of the technology are described further in the following description, examples, claims and drawings.

### BRIEF DESCRIPTION OF THE DRAWINGS

The drawings illustrate embodiments of the technology and are not to be construed as limiting thereof. For clarity and ease of illustration, the drawings are not made to scale, and, in some instances, various aspects may be shown exaggerated or enlarged to facilitate an understanding of particular embodiments.

FIG. 1 shows a box-plot representation of variability in gene fold expression [2^{-(ΔΔCt)}] in between amyotrophic lateral sclerosis (ALS, n=119), Parkinson's disease (PD, n=20), primary lateral sclerosis (PLS, n=42), and control samples (n=150) represented by boxplot for eight-miRNAs. Center lines show the medians; box limits indicate the 25th and 75th percentiles; whiskers extend 1.5 times the interquartile range from the 25th and 75th percentiles, outliers are represented by circles. Two extreme values (282 and 275) from control samples were removed from miR-4454 to best illustrate the data. See Table 3 for statistical analyses.

FIG. 2 is a Receiver Operator Characteristics (ROC) curve calculated with a logistic regression model on 269 total observations with 119 ALS and 150 control samples. Area under the curve (AUC)=0.98.

FIG. 3 shows a comparison between real-time PCR (qPCR) and digital PCR (dPCR) for the eight miRNAs of Example 2.

FIG. 4 shows the box and whisker plots of 8-miRNA included as variables in a cross-validation logistic regression model for an ALS diagnostic test based on 788 ALS patients and controls. The center line represents the median of each group. The box represents the 25th and 75th percentiles. The whiskers depict 1.5 times each inter-quartile range. The outliers are shown as circles

### DETAILED DESCRIPTION

As described herein, the miRNA content of circulating bodily fluids has diagnostic and therapeutic utility. The identification of the specific miRNA useful in detecting the presence of, or the risk of developing any motor neuron disease and/or neurodegenerative disorder or certain motor neuron diseases and/or neurodegenerative disorders, such as ALS, PLS, AD, MCI, PD, MS, HD, CJD, LBD, MSA, FTD, and PSP can be determined by methods disclosed in, for example, U.S. Patent Application Publication No. 2021/0164051, entitled: “Methods of Detection and Analysis of Nucleic Acid in Neural-Derived Exosomes,” the disclosure of which is incorporated by reference herein in its entirety. While the use of exosomes derived from neural cells is useful in the initial identification of miRNA and analyzing the relationships between healthy and un-healthy individuals, using exosomes derived from neural cells in a diagnostic method is complex and cumbersome. The inventors discovered, quite unexpectedly, that once the plurality of miRNA were identified using the NEE method, detecting the onset or progression or risk of developing a motor neuron disease and/or neurodegenerative disorder by determining a presence or amount of one or more micro RNAs (miRNAs) in a circulating bodily fluid sample (e.g., blood) without isolating and assessing the presence or amount of one or more miRNAs in neural-derived exosomes, was more robust and accurate than determining a presence or amount of one or more miRNAs in exosomes derived from neural cells. Thus, the diagnostic methods described herein, once developed using the methods described herein which include isolating and assessing the presence or amount of one or more miRNAs in neural-derived exosomes as an initial process, are not only simpler and easier than using exosomes derived from neuronal cells, but they are unexpectedly more robust, accurate and predictive.

As discussed above, the techniques described in U.S. Patent Application Publication No. 2021/0164051, can be useful in determining the one or more miRNAs that could be used to identify a subject who has, or is at risk of developing a motor neuron disease and/or neurodegenerative disorder. The abundance of, for example, tetraspanins and cell adhesion molecules (CAMs) expressed on or in exosomes derived from neural cells or neural tissues makes it possible to detect, enrich, prepare, and/or isolate neural-derived exosomes. Such neural-derived exosomes may be employed to detect and/or quantify the amount of certain exosome-derived miRNAs. The presence or amount of certain exosome-derived miRNAs, or sets of such miRNAs, provides insight as to whether a subject has, or is at risk of developing any motor neuron disease and/or neurodegenerative disorder, or whether a subject has, or is at risk of developing certain motor neuron diseases and/or neurodegenerative disorders. For example, the presence or amount of certain exosome-derived miRNAs in combination can also be used to provide early diagnosis of a motor neuron disease and/or neurodegenerative disorders and/or to identify subjects who are at risk of developing the motor neuron disease and/or neurodegenerative disorders. Once one or more miRNAs have been identified using exosomes as being useful in identifying a subject who has or is at risk of developing a motor neuron disease and/or neurodegenerative disorders, using the guidelines provided herein, the one or more miRNAs so identified, then can be detected (presence and/or amount) in circulating bodily fluids such as blood without isolating exosomes, and the presence or amount of the miRNAs used to identify such subjects. Such diagnostic methods are far simpler to carry out using circulating bodily fluids, and can be more robust and more accurate, when compared to using exosomes derived from neuronal cells.

Amyotrophic Lateral Sclerosis (ALS), primary lateral sclerosis (PLS), Alzheimer's Disease (AD), Mild Cognitive Impairment (MCI), Parkinson's Disease (PD), Huntington's Disease (HD), prion diseases such as Creutzfeld-Jakob disease (CJD), and Progressive Supranuclear Palsy (PSP), Lewy Body Disease (LBD), Multiple System Atrophy (MSD, and Frontotemporal Dementia (FTD), non-limiting examples of motor neuron diseases and/or neurodegenerative disorders, are typically progressive diseases and disorders that are not readily diagnosed. There are a variety of genetic and environmental risk factors that may lead to ALS, PLS, AD, MCI, PD, MS, HD, CJD, PSP, LBD, MSA, and FTD, and others, which are believed to result from gene/environment interactions and these diseases and/or disorders likely represent syndromes rather than a single disease. The onset of ALS, PLS, AD, MCI, PD, MS, HD, CJD, PSP, LBD, MSA, and/or FTD, and others may present a crisis for patients and their families. Loss of functionality due to progressive motor neuron loss leads to ataxia, aphasia, muscle spasticity, muscle fasciculations, and progressive paralysis. Some patients also suffer cognitive deficits.

One of the problems in current motor neuron disease and/or neurodegenerative disorder (including, for example, ALS, PLS, AD, MCI, PD, MS, HD, CJD, PSP, LBD, MSA, and FTD) therapies is protracted diagnosis. In their initial presentations, patients are often misdiagnosed. Few general practitioners feel comfortable in making a possible or probable diagnosis of these types of diseases and disorders, and often refer patients to neurologists, who in turn, typically use progressive deterioration of upper and lower motor neuron function, increasing muscle atrophy as measured through time, and other methodologies as indicative of ALS, PLS, AD, MCI, PD, MS, HD, CJD, PSP, LBD, MSA, and FTD. As a result, diagnosis of a motor neuron disease and/or neurodegenerative disorder, including specific diagnosis of ALS, PLS, AD, MCI, PD, MS, HD, CJD, PSP, LBD, MSA, and FTD typically takes months, and sometimes a year or more, to be made. This precious time lost due to the inability to diagnose presents a significant psychological burden on patients and their families, as well as their physicians, because of the inability to prescribe medication or even to plan for treatment and patient care.

In one embodiment, there is provided a motor neuron disease and/or neurodegenerative disorder diagnostic multi-miRNA fingerprint (one or more, two or more, three or more, four or more, five or more, up to all of miRNA in each set corresponding to the specific motor neuron disease and/or neurodegenerative disorder), obtained from a standard clinical blood draw that may be useful in detecting and/or diagnosing the presence of, or risk of developing a motor neuron disease and/or neurodegenerative disorder. In an embodiment, the one or more motor neuron diseases and/or neurodegenerative disorders is selected from ALS, PLS, AD, MCI, PD, MS, HD, CJD, PSP, LBD, MSA, and FTD although the methods and techniques described herein can be used to generate larger miRNA screening panels for additional diseases and disorders, and uses of those miRNA screening panels. Each miRNA were identified from an earlier experiment on blood plasma from neural enriched extracellular vesicles in which the miRNA were shown by next generation sequencing to be differentially expressed between motor neuron disease and/or neurodegenerative disorder patients and healthy controls.

In this embodiment, the method of developing an miRNA screening panel may include isolating neural-enriched extracellular vesicles (NEEs) or exosomes from test samples from subjects who were diagnosed as having at least one motor neuron disease or neurodegenerative disorder, detecting and/or quantifying the amount of exosome-derived miRNAs for each disease or disorder that the respective subject was previously diagnosed with, comparing the presence and/or amount of exosome-derived miRNAs from the test samples from subjects who were diagnosed as having at least one motor neuron disease or neurodegenerative disorder with the same miRNAs from a sample from a healthy subject, and selecting those exosome-derived miRNAs for each disease or disorder whose presence and/or amount are statistically significantly different from the miRNA of healthy subjects, typically by being present in an amount of greater than 1.1, such as from about 1.1 to more than 20-fold higher or lower than the miRNA of a healthy subject. In accordance with this method, a plurality of miRNAs will be separately identified for each of the motor neuron diseases and/or neurodegenerative disorders, such as but not limited to ALS, PLS, AD, MCI, PD, MS, HD, CJD, PSP, LBD, MSA, and FTD thereby providing an exosome-derived miRNA screening panel.

The method of developing an miRNA screening panel may further comprise carrying out the same comparison as above, except the miRNA are isolated and detected directly from a test sample of a subject's blood, the subject having been previously diagnosed with at least one motor neuron disease and/or neurodegenerative disorder, without isolation of NEEs or exosomes, (e.g., not making use of the optional first stage), comparing the amounts or presence of each miRNA with control or healthy subject's miRNA, and selecting those miRNAs for any motor neuron disease or disorder, or selecting those miRNAs for each disease or disorder whose presence and/or amount are statistically significantly different from the miRNA of healthy subjects, typically by being present in an amount greater than 1.1, such as from about 1.1 to greater than 20-fold higher or lower than the miRNA of a healthy subject. In accordance with this method, a plurality of miRNAs will be separately identified for any motor neuron disease and/or neurodegenerative disorder in general, or for each of ALS, PLS, AD, MCI, PD, MS, HD, CJD, PSP, LBD, MSA, and FTD thereby providing a non-exosome-derived miRNA screening panel.

The method may optionally further include comparing the plurality of miRNAs in the exosome-derived miRNA screening panel (if the first stage were carried out) with the plurality of miRNAs in the non-exosome-derived miRNA screening panel, and selecting a plurality of miRNA for any motor neuron disease and/or neurodegenerative disorder in general, or for each of ALS, PLS, AD, MCI, PD, MS, HD, CJD, PSP, LBD, MSA, and FTD that are similarly detected and differentially expressed in each panel. Similarly detected and differentially expressed denotes the respective miRNA are present in an amount greater than 1.1, such as from about 1.1 to greater than 20-fold higher or lower than the control, and the respective miRNA are both either higher or lower. The selection of the plurality of miRNA for any motor neuron disease and/or neurodegenerative disorder in general, or for each of ALS, PLS, AD, MCI, PD, MS, HD, CJD, PSP, LBD, MSA, and FTD provides a final miRNA screening panel that can be used in the methods described further hereinbelow. Using the guidelines provided herein, the final miRNA screening panel can be periodically updated to include additional motor neuron diseases, and/or neurodegenerative disorders.

The method of developing an miRNA screening panel for any motor neuron disease and/or neurodegenerative disorder in general, or for a plurality of motor neuron diseases and/or neurodegenerative disorders can therefore comprise: (1) optionally developing an exosome-derived miRNA screening panel for any motor neuron disease and/or neurodegenerative disorder in general, or for a plurality of motor neuron diseases and/or neurodegenerative disorders; (2) developing a non-exosome-derived miRNA screening panel for any motor neuron disease and/or neurodegenerative disorder in general, or for a plurality of motor neuron diseases and/or neurodegenerative disorders; (3) optionally comparing the miRNA screening panels developed in (1) and (2) and optionally selecting miRNA that are similarly present in both (1) and (2) to provide a final miRNA screening panel.

Development of an miRNA screening panel for ALS and/or PLS is described in, for example, U.S. patent application Ser. No. 18/794,425, filed on Dec. 9, 2024, entitled: “Methods of Detection and Analysis of Nucleic Acid in Circulating Bodily Fluids,” as well as U.S. Patent Application Publication No. 2021/0164051, the disclosures of each of which are incorporated by reference herein in their entireties. U.S. Patent Application Publication No. 2021/0164051 discloses the development of an exosome-derived miRNA screening panel for ALS and/or PLS, and U.S. patent application Ser. No. 18/794,425 discloses the development of a non-exosome-derived miRNA screening panel for ALS and/or PLS, comparing that panel with the exosome-derived screening panel, and development of a final miRNA screening panel for ALS and/or PLS. In accordance with these disclosures, an eight-miRNA fingerprint or panel was developed that was useful in diagnosing ALS and/or PLS. See, e.g., Banack S A, et al., “An miRNA fingerprint using neural-enriched extracellular vesicles from blood plasma: Towards a biomarker for amyotrophic lateral sclerosis/motor neuron disease,” Open Biology. 10(6):200116 (2020). Thirty-four miRNAs were selected, due to their magnitude of dysregulation, for validation using qPCR. The eight-miRNA were selected from these 34 miRNAs because they consistently and statistically separated ALS and/or PLS patients and control patients following qPCR experiments on three different cohorts of patient and control samples. Banack S A, et al., “miRNA extracted from extracellular vesicles is a robust biomarker of amyotrophic lateral sclerosis,” J. Neurol Sci. 442:120396 (2022). The bilipid membrane of the extracellular vesicles contributed to reproducibility in two ways: 1) protecting miRNA from degradation, and 2) facilitating purification of the sample by immunopurification through transmembrane proteins on the surface of the extracellular vesicles. Dunlop R A, et al., “L1CAM immunocapture generates a unique extracellular vesicle population with a reproducible miRNA fingerprint,” RNA biology, 20(1):140-148 (2023).

Using the techniques disclosed therein, along with the guidance provided herein, similar methodologies can be employed for additional neurodegenerative disorders, such as AD, MCI, PD, MS, HD, CJD, PSP, LBD, MSA, and FTD, to develop respective final screening panels for each of these disorders, and to develop a final screening panel for any motor neuron disease and/or neurodegenerative disorder. A final array then may be developed for any motor neuron disease and/or neurodegenerative disorder in general, or for a plurality of neurodegenerative diseases and/or disorders, including for example, ALS, PLS, AD, MCI, PD, MS, HD, CJD, LBD, MSA, FTD, and PSP, that can be used to provide an miRNA screening panel for a plurality of neurodegenerative disease and disorders. The development and use of a motor neuron disease and/or neurodegenerative disorder panel containing specific miRNAs, or subsets thereof, along with their respective coefficients indicative of each miRNA's contribution to the diagnosis, can be predictive and/or diagnostic of any motor neuron disease and/or neurodegenerative disorder in general, or of one or more motor neuron diseases and/or neurodegenerative disorders selected from at least amyotrophic lateral sclerosis (ALS), primary lateral sclerosis (PLS), Alzheimer's Disease (AD), Mild Cognitive Impairment (MCI), Parkinson's Disease (PD), Multiple Sclerosis (MS), Huntington's Disease (HD), prion diseases such as Creutzfeld-Jakob disease (CJD), Progressive Supranuclear Palsy (PSP), Lewy Body Disease (LBD), Multiple System Atrophy (MSA), and Frontotemporal Dementia (FTD) can be useful in diagnosing the mere presence of a disease or disorder, or specifically diagnosing ALS, PLS, AD, MCI, PD, MS, HD, CJD, PSP, LBD, MSA, and/or FTD, determining subjects at risk of developing any motor neuron disease and/or neurodegenerative disorder in general, or of developing ALS, PLS, AD, MCI, PD, MS, HD, CJD, LBD, MSA, FTD, and/or PSP, testing subject to exclude a future motor neuron disease and/or neurological disorder, ALS, PLS, AD, MCI, PD, MS, HD, CJD, LBD, MSA, FTD, and/or PSP diagnosis, and in assessing therapies for ALS, PLS, AD, MCI, PD, MS, HD, CJD, LBD, MSA, FTD, and/or PSP. Table 1 below lists exemplary miRNA identified using the techniques described herein for developing final miRNA panels for each of the respective diseases and/or disorders listed therein. Table 2 lists exemplary miRNA identified using the techniques described herein for developing a final miRNA panel for any motor neuron diseases and/or neurodegenerative disorder.

TABLE 1
Motor Neuron Disease or Neurological Disorder

| ALS | PLS | AD | MCI | PD | MS | HD | CJD | PSP |
|---|---|---|---|---|---|---|---|---|
| 151a-3p | let-7c-5p | 151a-3p | 151a-3p | 146a-5p | 145 | 9 | let-7i-5p | 22-3p |
| 151a-5p | | 151a-5p | 151a-5p | | 320c | 10b-5p | 16-5p | 132-3p |
| 146a-5p | 146a-5p | | 146a-5p | | 23a-3p | 146a | 93-5p | 146a-5p |
| 4454 | | | 4454 | 150-5p | 326 | 196a | 106b-3p | 455-5p |
| 10b-5p | 10b-5p | 10b-5p | 10b-5p | | 150-5p | 214 | 129-1-3p | 132 |
| 199a-3p | | | 199a-3p | | 320a-3p | 520f-3p | 129b-5p | 124 |
| 199a-5p | 199a-5p | 199a-5p | 199a-5p | | 155a-5p | 135b-3p | 337-3p | 147a |
| 29b-3p | | | 29b-3p | | 146a-5p | 4317 | let-7c-2-3p | 518e |
| 1290 | 150-5p | | 126-5p | 126-3p | 34a-3p | 3928-5p | 133a-3p | 19b-3p |
| 99a-5p | 153-3p | 150-5p | 150-5p | | 143a-3p | 8082 | 181a-5p | 33a-5p |
| 150-5p | 155-5p | 107 | 107 | | 373a-5p | 140-5p | 1a-3p | 130b-3p |
| 155-5p | | | let-7g-5p | 148b-3p | 153-3p | | 30a-3p | 136-3p |
| 223-3p | | 1290 | 1290 | | 1290 | | 3102-3p | 210-3p |
| 486-5p | | | 95-3p | | 375-3p | | 365-3p | |
| 320c | 19b-3p | 375-3p | 375-3p | | 99a-5p | | 455-3p | |
| 148b-3p | 223-3p | 92a-1-5p | 99a-5p | | 342-3p | | 505-5p | |
| let-7c-5p | | | 125b-5p | | 486-5p | | 6240 | |
| 30d-5p | | | 642a-5p | | 320b | | 328-3p | |
| 320b | | | 92a-1-5p | | 148b-3p | | 101-3p | |
| 342-3p | | | 362-5p | 375-3p | let-7c-5p | | 10a-5p | |
| 126-3p | 320b | 342-3p | 342-3p | 223-3p | 376c-3p | | 142-3p | |
| 25-3p | 320c | | 196a-5p | 486-5p | 154-5p | | 223-3p | |
| 652-3p | | | 155-5p | 376c-3p | 142-3p | | 296-5p | |
| 376c-3p | | | 409-3p | 154-5p | 103a-3p | | 370-3p | |
| 142-3p | 342-3p | 744-5p | 744-5p | 142-3p | 10b-5p | | | |
| 451a | | 223-3p | 223-3p | 107 | 199a-5p | | | |
| 126-5p | | | 584-5p | 4454 | 362-5p | | | |
| 335-5p | | | 340-5p | 10b-5p | 584-5p | | | |
| let-7g-5p | | | 421 | 1290 | 340-5p | | | |
| 125b-5p | | 486-5p | 486-5p | 95-3p | 30d-5p | | | |
| | 1290 | | 126-3p | 99a-5p | 381-3p | | | |
| | 99a-5p | 320c | 320c | 362-5p | 151a-3p | | | |
| | 486-5p | 320b | 320b | 155-5p | let-7g-5p | | | |
| | 142-3p | | let-7d-3p | 320c | 642a-5p | | | |
| | 148b-3p | | 27b-3p | 320b | 223-3p | | | |
| | | | 148b-3p | 25-3p | 652-3p | | | |
| | | | 143-3p | 153-3p | 451a | | | |
| | | | 326 | let-7c- | 30c-5p | | | |
| | | | | 5p | | | | |
| | | | 335-5p | 652-3p | 4454 | | | |
| | | 25-3p | 25-3p | 381-3p | | | | |
| | | 153-3p | 153-3p | 103a-3p | | | | |
| | | 19b-3p | 19b-3p | | | | | |
| | | let-7c-5p | let-7c-5p | | | | | |
| | | 652-3p | 652-3p | | | | | |
| | | | 376c-3p | | | | | |
| | | | 154-5p | | | | | |
| | | | 376a-3p | | | | | |
| | | 30d-5p | 30d-5p | | | | | |
| | | 381-3p | 381-3p | | | | | |
| | | 451a | 451a | | | | | |
| | | 142-3p | 142-3p | | | | | |
| | | | 103a-3p | | | | | |
| | | | 23a-3p | | | | | |
| | | | 30c-5p | | | | | |

TABLE 2

| 151a-3p | let-7g-5p | 155-5p | let-7d-3p | 376c-3p |
|---|---|---|---|---|
| 151a-5p | 1290 | 409-3p | 27b-3p | 154-5p |
| 146a-5p | 95-3p | 744-5p | 148b-3p | 376a-3p |
| 4454 | 375-3p | 223-3p | 143-3p | 30d-5p |
| 10b-5p | 99a-5p | 584-5p | 326 | 381-3p |
| 199a-3p | 125b-5p | 340-5p | 335-5p | 451a |
| 199a-5p | 642a-5p | 421 | 25-3p | 142-3p |
| 29b-3p | 92a-1-5p | 486-5p | 153-3p | 103a-3p |
| 126-5p | 362-5p | 126-3p | 19b-3p | 23a-3p |
| 150-5p | 342-3p | 320c | let-7c-5p | 30c-5p |
| 107 | 196a-5p | 320b | 652-3p | |

The miRNA identified in the above tables are exemplary and not limiting. The miRNA listed in Table 2, the miRNA listed in Table 1 for ALS, PLS, AD, MCI, and PD were all determined using the techniques described herein, and the miRNA listed in Table 1 for MS, HD, CJD, and PSP were taken from published literature and have not been verified using the methods described herein. Those skilled in the art will be capable of determining and confirming a suitable suite or panel of miRNAs for each of the above listed diseases/disorders using the guidelines provided herein, and will be capable of determining and confirming a suitable suite or panel of miRNAs for additional diseases/disorders to increase the breadth of the final miRNA panel. For each disease/disorder in the panel, the methods of diagnosis, assessing disease progression, and assessing treatments will vary depending on the total number of miRNAs needed to confirm a statistically meaningful diagnosis. For example, the inventors have determined that, for ALS, anywhere from 2 to 8, 3 to 8 of the first 8 miRNA listed in the above table, preferably 4 to 8, or 5 to 8, or 6 to 8, or 7 to 8, or all 8, are identified in a subject's blood as up or down regulated, and used to ensure a statistically meaningful diagnosis with greater than 90% accuracy, preferably greater than 95% accuracy. The same or similar analysis can be carried out for each of the diseases/disorders in the final miRNA panel and the appropriate number and type of miRNA determined to ensure a statistically meaningful diagnosis for each disease/disorder, and to differentiate between the respective diseases/disorders. Accordingly, a subject suspected of having a motor neuron disease and/or neurodegenerative disorder may be tested by withdrawing a sample from the subject, analyzing the miRNA in the sample, conducting a comparison of the miRNA identified by presence and amount, (including direction of fold regulation), and diagnosing the presence or absence of any motor neuron disease and/or neurodegenerative disorder (Table 2), or diagnosing the presence or absence of one or more of the motor neuron diseases and/or neurodegenerative disorders (Table 1).

The identified final miRNA panel fills an unmet motor neuron disease and/or neurodegenerative disorder drug development and medical diagnostic need and could facilitate the identification of any motor neuron disease and/or neurodegenerative disorder or one or more of ALS, PLS, AD, MCI, PD, MS, HD, CJD, and/or PSP, or subjects at risk of developing any motor neuron disease and/or neurodegenerative disorder, or one or more of ALS, PLS, AD, MCI, PD, MS, HD, CJD, LBD, MSA, FTD, and/or PSP, at its earliest stages, thereby reducing diagnostic uncertainty. The inventors surprisingly discovered that the final miRNA panel can be used to test a subject to diagnose or to exclude a future diagnosis of any motor neuron disease and/or neurodegenerative disorder, or of one or more of ALS, PLS, AD, MCI, PD, MS, HD, CJD, LBD, MSA, FTD, and/or PSP, from a standard clinical blood draw, without the need to isolate exosomes.

A “sample” or “samples”, as used interchangeable herein, is often obtained from a suitable subject. A sample can be isolated or obtained directly from a subject or part thereof. In some embodiments, a sample obtained from a subject is a sample derived from the subject. Accordingly, in certain embodiments, a sample obtained from a subject is a sample obtained directly from the subject. In certain embodiments, a sample obtained from a subject is obtained from a third party, for example a third party who obtained or extracted the sample from the subject. In some embodiments, a sample is obtained indirectly from an individual or a medical professional. A sample can be any specimen that is isolated or obtained from a subject or part thereof. Non-limiting examples of samples include fluids or tissues obtained or derived from a subject, including, without limitation, circulating bodily fluids such as blood or a blood product (e.g., serum, plasma, platelets, buffy coats, lymphatic fluid or the like), umbilical cord blood, chorionic villi, amniotic fluid, cerebrospinal fluid (CSF), spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells, neurons) or parts thereof (e.g., mitochondrial, nucleus, extracts, lysates, or the like), urine, feces, sputum, saliva, nasal mucous, prostate fluid, lavage, semen, lymphatic fluid, bile, tears, sweat, breast milk, breast fluid, the like or combinations thereof. In an embodiment, a “sample” is blood.

As used herein, the expressions “circulating bodily fluid” or “circulating body fluid” denotes samples, as described above, that circulate throughout the body. Particularly preferred circulating bodily fluids include, but are not limited to, blood, serum, plasma, platelets, buffy coats, lymphatic fluid, urine, semen, spinal fluid, bile, and the like. Blood is especially preferred due to its ease of sampling.

Non-limiting examples of subjects include mammals, humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animals (e.g., mouse, rat, rabbit, and guinea pig). In some embodiments a subject is a mammal. A mammal can be any age or at any stage of development (e.g., an adult (e.g., 18, 19, 20 or 21 years and older), a senior adult (e.g., over the age of 55, over the age of 60, or over the age of 65 years), a teen (e.g., age 12 to 19 yrs.), child (e.g., age 1 to 12 yrs.), infant (e.g., from birth to 1 yr.), or a mammal in utero). A mammal can be male or female. In some embodiments, a subject is a human.

In some embodiments, a subject has, is suspected of having, or is at risk of developing, any motor neuron disease and/or a neurodegenerative disorder. In some embodiments the motor neuron disease and/or a neurodegenerative disorder is selected from one or more of ALS, PLS, AD, MCI, PD, MS, HD, CJD, LBD, MSA, FTD, and/or PSP. In some embodiments, a subject who has a motor neuron disease and/or neurodegenerative disorder is a subject diagnosed as having such a condition by a medical professional (e.g., a medical doctor) based on, for example the presence of one or more diagnostic symptoms and/or the results of one or more standardized diagnostic test results. A subject suspected of having a motor neuron disease and/or neurodegenerative disorder is a subject not yet diagnosed as having such a condition by a medical professional. In some embodiments, a subject suspected of having a motor neuron disease and/or neurodegenerative disorder may display one or more symptoms of such a condition, which symptoms are not conclusive evidence that the subject has the condition. In some embodiments, a subject who is suspected of a having a motor neuron disease and/or neurodegenerative disorder may have one or more symptoms of such a condition, but is not diagnosed as having any one particular motor neuron disease and/or neurodegenerative disorder because there is not enough data to indicate conclusively that the subject has a particular condition. In some embodiment, a subject who is suspected of having a motor neuron disease and/or neurodegenerative disorder is a subject suspected of having ALS, PLS, AD, MCI, PD, MS, HD, CJD, LBD, MSA, FTD and/or PSP, but is not diagnosed as having ALS, PLS, AD, MCI, PD, MS, HD, CJD, LBD, MSA, FTD and/or PSP by a medical professional.

In some embodiments, a subject is determined to be a subject at risk of developing a motor neuron disease and/or neurodegenerative disorder by carrying out one or more of the methods described herein. In some embodiments, a subject at risk of developing a motor neuron disease and/or neurodegenerative disorder is a subject who is asymptomatic for a motor neuron disease and/or neurodegenerative disorder. In some embodiments, a subject at risk of developing a motor neuron disease and/or neurodegenerative disorder is a subject having one or more symptoms of a motor neuron disease and/or neurodegenerative disorder, which symptoms may be mild or transient in nature. In some embodiments, a subject at risk of developing a motor neuron disease and/or neurodegenerative disorder is a subject who is not yet diagnosed as having a motor neuron disease and/or neurodegenerative disorder. In some embodiments, a subject at risk of developing a motor neuron disease and/or neurodegenerative disorder is a subject suspected of having a motor neuron disease and/or neurodegenerative disorder. In additional embodiments, a subject is a healthy individual tested as a preventative screen to rule out the possibility of being at risk of developing a motor neuron disease and/or neurodegenerative disorder and to identify the possibility of onset at an early stage before symptoms arise.

In certain embodiments, the presence or absence of any motor neuron disease and/or neurodegenerative disorder in general, or the presence or absence of ALS, PLS, AD, MCI, PD, MS, HD, CJD, LBD, MSA, FTD, and/or PSP in a subject is determined by the methods described herein. In some embodiments, wherein a subject is determined to have any motor neuron disease and/or neurodegenerative disorder in general, or to have ALS, PLS, AD, MCI, PD, MS, HD, CJD, LBD, MSA, FTD, and/or PSP that is diagnosed by one or more of the methods described herein, there is further provided a method of treating the disease or disorder in general, or in treating ALS, PLS, AD, MCI, PD, MS, HD, CJD, LBD, MSA, FTD and/or PSP by administering a suitable treatment to the subject, wherein the disease or disorder, or one or more symptoms thereof are therapeutically treated. In certain embodiments, the methods described herein identify a subject who is at risk of developing any motor neuron disease and/or neurodegenerative disorder in general, or ALS, PLS, AD, MCI, PD, MS, HD, CJD, LBD, MSA, FTD and/or PSP. In some embodiments, wherein a subject is identified as at risk of developing any motor neuron disease and/or neurodegenerative disorder in general, or ALS, PLS, AD, MCI, PD, MS, HD, CJD, LBD, MSA, FTD, and/or PSP by the methods described herein, there is further provided a method of treating that subject by administering a suitable treatment to the subject, wherein development of any motor neuron disease and/or neurodegenerative disorder in general, or ALS, PLS, AD, MCI, PD, MS, HD, CJD, LBD, MSA, FTD, and/or PSP is prevented or delayed, or wherein one or more symptoms thereof are therapeutically treated. In some embodiments, a method of treating ALS, PLS, AD, MCI, PD, MS, HD, CJD, LBD, MSA, FTD, and/or PSP is a method of inhibiting or delaying the onset or progression of ALS, PLS, AD, MCI, PD, MS, HD, CJD, LBD, MSA, FTD, and/or PSP, for example in a subject at risk of developing ALS, PLS, AD, MCI, PD, MS, HD, CJD, LBD, MSA, FTD, and/or PSP. In some embodiments, a method comprises treating ALS, PLS, AD, MCI, PD, MS, HD, CJD, LBD, MSA, FTD, and/or PSP or one or more symptoms thereof. In some embodiments, a method of treating ALS, PLS, AD, MCI, PD, MS, HD, CJD, LBD, MSA, FTD, and/or PSP is a method of inhibiting or delaying the onset or progression of one or more symptoms of ALS, PLS, AD, MCI, PD, MS, HD, CJD, LBD, MSA, FTD, and/or PSP, for example in a subject at risk of developing ALS, PLS, AD, MCI, PD, MS, HD, CJD, LBD, MSA, FTD, and/or PSP.

Non-limiting examples of a symptom of a motor neuron disease include a motor deficiency; fatigue (e.g., excessive fatigue); passivity; lethargy; inertia; tremors; ataxia; speaking difficulty (e.g., slurred, thick or irregular speech); muscle cramps (e.g., excessive muscle cramping, not necessarily induced by excessive use or excessive exercise), twitching, atrophy or weakness; shortness of breath; breathing difficulty; writing difficulty; unusual or frequent stiffness or rigidity; loss of fine or gross motor control; slowing of movement; impaired balance; body instability; posture or gait abnormality (e.g., shuffling walk, unsteady or irregular gait); reduced coordination; motor dysfunction; jerky or involuntary body movement; slowed saccadic eye movement; seizures; difficulty chewing, eating, or swallowing; loss of balance; ophthalmoparesis or impaired eye movement; impaired eyelid function; involuntary facial muscle contracture; neck dystonia or backward tilt of the head with stiffening of neck muscles; urinary/bowel incontinence; parkinsonism; the like and combinations thereof.

In some embodiments, a method comprises preventing or treating the afore-mentioned motor neuron diseases and/or neurodegenerative disorders, inhibiting or delaying the onset of, or progression of them, or inhibiting, mitigating, reducing or delaying the onset of one or more symptoms of them, where the method comprises administering a therapeutically effective amount of a drug or other therapy to treat the motor neuron disease and/or neurodegenerative disorder, non-limiting examples of which include L-serine, ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenylbut, imagabalin, atagabalin, 4-methylpregabalin, PD-217,014, Riluzole, Edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine (e.g., an immunogenic amount of an amyloid peptide, or a fragment or variant thereof, with or without an adjuvant), a cholinesterase inhibitor (e.g., donepezil, galantamine or rivastigmine), memantine, an antidepressant, an N-methyl D-aspartate (NMDA) antagonist, an omega-3 fatty acid, curcumin, or a curcumin derivative, vitamin E, a sleep aid (e.g., zolpidem, eszopiclone or zaleplon), an anti-anxiety drug (e.g., lorazepam and clonazepam), an anti-convulsant (e.g., sodium valproate, carbamazepine, or oxcarbazepine), an anti-psychotic (e.g., risperidone, quetiapine or olanzapine), carbidopa-levodopa, amantadine, a dopamine agonists (e.g., pramipexole, ropinirole, rotigotine or Apomorphine), a MAO B inhibitor (e.g., selegiline, rasagiline or safinamide), a Catechol O-methyltransferase (COMT) inhibitors (e.g., entacapone or tolcapone), an anticholinergic (e.g., benztropine or trihexyphenidyl), the like and combinations thereof. In some embodiments, a subject diagnosed as having ALS, or at risk of developing ALS and/or PLS, is treated by a method comprising administering a therapeutically effective amount of one or more of L-serine, Riluzole, Edaravone, Nusinersen, Onasemnogene abeparvovec-xioi (ZOLGENSMA™), Radicava, Rilutek, Tiglutik, Nuedexta, muscle relaxers (e.g., baclofen, tizanidine, benzodiazepines) or botulinum toxin. In some embodiments, a subject diagnosed as having ALS and/or PLS, or at risk of developing ALS and/or PLS is treated by a method comprising administering a therapeutically effective amount of L-serine. Known drugs or therapies useful in treating the motor neuron disease and/or neurodegenerative disorder, and experimental drugs or therapies can be administered and the efficacy assessed using the miRNA panel disclosed herein.

In some embodiments, a suitable method of treatment comprises administering a therapeutically effective amount of ralitoline, phenytoin, lamotrigine, carbamazepine, lidocaine, tetrodotoxin, Riluzole, Edaravone, Gabapentin, pregabalin, Mirogabalin, gabapentin enacarbil, phenylbut, imagabalin, atagabalin, 4-methylpregabalin, PD-217,014, Trihexyphenidyl, amitriptyline, baclofen, diazepam, L-serine, CK-2127107 (reldesemtiv), Nusinersen, Onasemnogene abeparvovec-xioi (ZOLGENSMA™), Radicava, Rilutek, Tiglutik, Nuedexta, the like or combinations thereof. In some embodiments, a suitable method of treatment comprises administering a therapeutically effective amount of L-serine.

### L-Serine

In some embodiments, a subject is administered a therapeutically effective amount of L-serine, a salt, metabolic precursor, derivative or conjugate thereof. In some embodiments, a subject is administered a therapeutically effective amount of free L-serine, or a salt thereof. A therapeutically effective amount of L-serine or free L-serine may be administered as a pharmaceutical composition comprising one or more pharmaceutical excipients, additives, carriers and/or diluents. In some embodiments, a method herein comprises administered a therapeutically effective amount of a composition comprising, consisting of, or consisting essentially of L-serine, a salt, metabolic precursor, derivative or conjugate thereof to a subject. In some embodiments, a method herein comprises administered a therapeutically effective amount of a composition comprising, consisting of, or consisting essentially of free L-serine, or a salt, derivative or conjugate thereof to a subject. In some embodiments, a method herein comprises administering a therapeutically effective amount of a composition comprising, consisting of, or consisting essentially of a polymer of L-serine, or a salt, derivative or conjugate thereof to a subject. In some embodiments, a composition consisting essentially of L-serine, free L-serine, or a salt, a precursor, a derivative or a conjugate thereof excludes proteins or protein fractions comprising less than 100%, 99%, 98%, less than 95%, less than 90%, less than 80%, less than 70%, less than 60%, or less than 50% L-serine (wt/wt). In some embodiments, a composition consisting essentially of L-serine, free L-serine, or a salt, a precursor, a derivative or a conjugate thereof excludes proteins or protein fractions comprising greater than 5%, greater than 10%, greater than 20%, greater than 30%, greater than 40%, greater than 50% or greater than 60% protein (wt/wt), and excludes other components that materially affect the therapeutic efficacy of the L-serine to prevent and/or treat ALS. In some embodiments, a composition consisting essentially of L-serine comprises free L-serine, or a polymer of L-serine having an amino acid content of L-serine of at least 100%, 99%, 98%, 95%, 90%, 85% or at least 80%. In some embodiments, a composition consisting essentially of L-serine excludes creatine, creatine pyruvate, guanidino-acetic acid (GA), glycocyamine, N-amidinoglycine, and salts or esters thereof. In some embodiments, a composition consisting essentially of L-serine is a composition comprising free L-serine at a purity of at least 85%, at least 90%, at least 95%, at least 98%, at least 99% or 100%. In certain embodiments, a composition consisting essentially of L-serine, free L-serine, or a salt, a precursor, derivative or conjugate of L-serine, is a composition that also comprises zinc.

Free L-serine refers to L-serine in the form of a single amino acid monomer, or a salt thereof. In some embodiments, a composition comprises free L-serine at a purity of at least 85%, at least 90%, at least 95%, at least 98%, at least 99% or 100%. In certain embodiments, free L-serine is not covalently bonded to any other amino acid.

In some embodiments, a composition comprising L-serine may exclude other active ingredients. In some embodiments, a composition may exclude proteins containing L-serine. In some embodiments, a composition may exclude proteins having a molecular weight greater than 10 kDa, greater than 20 kDa, greater than 30 kDa or greater than 50 kDa. In some embodiments, a composition may exclude proteins containing less than 99%, 98%, 95%, 92%, 90%, 80%, 70%, 60%, or less than 50% L-serine. In some embodiments, a composition may exclude creatine, or any energy metabolism precursor of creatine, such as guanidino-acetic acid (GA), equivalents thereof, and mixtures thereof.

In certain embodiments, a composition comprises L-serine, non-limiting examples of which include free L-serine, and polymers or polypeptides comprising at least a 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% L-serine by weight or amino acid content. In some embodiments, a polymer of L-serine or a polypeptide comprising L-serine includes between 2 and 50000, between 2 and 500, between 2 and 100, between 2 and 50, between 2 and 20, between 2 and 15, between 2 and 10, between 2 and 9, between 2 and 8, between 2 and 7, between 2 and 6, between 2 and 5, or between 2 and 4 L-serine amino acids linked by covalent bonds. In certain embodiments, a composition comprises L-serine, non-limiting examples of which include a polymer or polypeptide comprising from 20% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, from 95% to 100%, from 96% to 100%, from 97% to 100%, from 98% to 100%, or from 99% to 100% content of L-serine (wt/wt) or amino acid content (i.e., L-serine monomers/total amino acid monomers).

Non-limiting examples of a salt of L-serine include a sodium salt, potassium salt, calcium salt, magnesium salt, zinc salt, ammonium salt; inorganic salts such as, hydrogen chloride, sodium chloride, potassium chloride, calcium chloride, sodium phosphate, potassium phosphate, and sodium hydrogen carbonate; organic salts such as, sodium citrate, citrate, acetate, and the like. In certain embodiments, a composition comprises L-serine as an alkylated L-serine, such as L-serine with an alkyl group, or e.g., an alkyl comprising 1-20 carbon atoms. In certain embodiments, a derivative of L-serine includes an L-serine ester, an L-serine di-ester, a phosphate ester of L-serine, or a sulfate or sulfonate ester of L-serine. Non-limiting examples of a conjugate of L-serine includes a pegylated L-serine (e.g., an L-serine comprising one or more polyethylene glycol (PEG) moieties), and a lipidated L-serine. Non-limiting example of a precursor of L-serine include L-phosphoserine.

Non-limiting examples of a precursor of L-serine include a pro-form of L-serine that is broken down into L-serine monomers by the digestive system of a subject. In some embodiments, L-serine or a conjugate thereof consists of a slow-release version. In some embodiments a derivative of L-serine is conjugated to a different molecule forming a prodrug from which L-serine is released after crossing the blood/brain barrier.

In some embodiments, a composition consisting essentially of L-serine may comprise some amount of D-serine. For example, a composition consisting essentially of L-serine may include a small amount of D-serine, for example, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% D-serine by weight (e.g., wt/wt) or amino acid content (e.g., L-serine/total amino acid content). For example, a composition may include from 0.001% to 30%, from 0.005% to 30%, from 0.1% to 30%, from 1% to 30%, from 2% to 30%, from 3% to 30%, from 4% to 30%, from 5% to 30%, from 6% to 30%, from 7% to 30%, from 8% to 30%, from 9% to 30%, from 10% to 30%, from 0.001% to 20%, from 0.005% to 0%, from 0.1% to 20%, from 1% to 20%, from 2% to 20%, from 3% to 20%, from 4% to 20%, from 5% to 20%, from 6% to 20%, from 7% to 20%, from 8% to 20%, from 9% to 20%, or from 10% to 20% D-serine. In some embodiments, a composition comprising or consisting essentially of L-serine, does not comprise a substantial amount of D-serine. In some embodiments, a composition comprising or consisting essentially of L-serine, does not contain D-serine.

### Methods Using Circulating Bodily Fluid

In some embodiments, a method presented herein detects or determines an amount of one or more miRNAs associated with any motor neuron disease and/or neurodegenerative disorder in general, or ALS, PLS, AD, MCI, PD, MS, HD, CJD, LBD, MSA, FTD, and/or PSP from circulating bodily fluid, for example, from a subject's blood, without isolating exosomes. The inventors' prior efforts carried out methods using exosomes, where the isolation, separation, identification, etc. of various miRNAs can be time consuming and cumbersome. While the use of exosomes is effective in identifying panels of miRNAs associated with any motor neuron disease and/or neurodegenerative disorder in general, or an identified motor neuron disorder such as ALS, PLS, AD, MCI, PD, MS, HD, CJD, LBD, MSA, FTD, and/or PSP, using exosomes as a commercial diagnostic poses some difficulties, including difficulty in automation, labor and time intensity, etc. It also would not have been known or appreciated that the same or all of miRNAs identified using neural-derived exosomes could also be detected in a subject's circulating body fluid without isolating and analyzing exosomes, nor would it have been expected that a diagnostic test using the same or all of these miRNAs from circulating bodily fluid would be as accurate or sensitive as a diagnostic test using neural-derived exosomes.

The inventors unexpectedly discovered that, with respect to ALS, the same miRNA can be found in circulating blood plasma without the neural-enriched or derived extracellular vesicles (NEE, exosomes) isolation and analysis steps, and that the same miRNA are more concentrated, providing a better signal for diagnostic applications. In some embodiments, determining the presence or amount of one or more miRNAs selected from one or more of miR-199a-3p, miR-4454, miR-10b-5p, miR-151a-5p, miR-199a-5p, miR-151a-3p, miR-146a-5p, and/or miR-29b-3p from a subject's circulating bodily fluid and the relative contribution of each constitutes a suitable method for diagnosing ALS, and thus can be used as one of the techniques in the final miRNA panel described herein. In some embodiments, determining the presence or amount and the relative combination which includes any of the following miRNAs: miR-199a-3p, miR-199a-5p, miR-126-5p, miR-150-5p, miR-99a-5p, miR-223-3p, miR-335-5p, miR-let-7g-5p, miR-let-7c-5p, miR-486-5p, miR-155-5p, miR-4454, miR-10b-5p, miR-1290, miR-146a-5p, miR-125b-5p, from a subject's circulating bodily fluid constitutes a suitable method for diagnosing ALS, and thus can be used as one of the techniques in the final miRNA panel described herein. In some embodiments, a method herein comprises determining the presence or amount of one or more miRNAs selected from one or more of those listed in the final miRNA panel from a subject's circulating bodily fluid prepared from a sample obtained from a subject that has, or is suspected of having, a motor neuron disease and/or neurodegenerative disorders. In some embodiments, a method herein comprises determining the presence or amount of one or more miRNAs selected from one or more of those listed in the final miRNA panel from a subject's circulating bodily fluid prepared from a sample obtained from a control subject. Non-limiting examples of a control subject include healthy subjects, a subject that does not have a motor neuron disease and/or a subject that is not suspected of having a motor neuron disease.

A feature of an embodiment includes a method of identifying a subject who has, or is at risk of developing any motor neuron disease and/or neurodegenerative disorder in general, or an identified motor neuron disease such as ALS, PLS, AD, MCI, PD, MS, HD, CJD, LBD, MSA, FTD, and/or PSP, the method comprising: (a) determining a presence or amount of one or more micro-RNAs (miRNAs) selected from the group consisting of those listed in the final miRNA panel from a subject's circulating bodily fluid without determining a presence or amount from a subject's neural-derived exosomes, and determining if the subject has, or is at risk of developing any motor neuron disease and/or neurodegenerative disorder in general, or the motor neuron disease such as ALS, PLS, AD, MCI, PD, MS, HD, CJD, LBD, MSA, FTD, and/or PSP, according to the presence or amount of the one or more miRNAs in the sample. In certain embodiments, the method comprises determining the presence or amount of two or more, three or more, four or more, five or more, six or more, seven or more, or all of the miRNAs listed for the specific motor neuron disease and/or neurodegenerative disorder in the final miRNA panel array (for ALS, the miRNAs would be selected from the group consisting of miR-199a-3p, miR-4454, miR-10b-5p, miR-151a-5p, miR-199a-5p, miR-151a-3p, miR-146a-5p, and miR-29b-3p; for any motor neuron disease or neurodegenerative disorder, the miRNAs would be selected from the group consisting of MiR-151a-3p. MiR-199a-5p, MiR-150-5p, MiR-1290, MiR-99a-5p, MiR-342-3p, MiR-223-3p, and MiR-652-3p). In certain embodiments, the presence of four or more, five or more, six or more, seven or more or all of the miRNAs listed for the specific motor neuron disease and/or neurodegenerative disorder in the final miRNA panel array in, on or within a subject's circulating bodily fluid obtained from a subject, indicates that the subject has or is at risk of having the so-identified motor neuron disease and/or neurodegenerative disorder.

In some embodiments, a method herein comprises comparing an amount of one or more miRNAs in circulating bodily fluid obtained from a sample from a control subject (e.g., a subject known to be free of a motor neuron disease) to an amount of one or more miRNAs in circulating bodily fluid obtained from a sample from a test subject (e.g., a subject suspected of having a motor neuron disease). In some embodiments, the presence or absence of a motor neuron disease and/or neurodegenerative disorder in a test subject is determined according to such a comparison, after generating qPCR data for each miRNA, generating normalized qPCR values for reach miRNA, and processing the normalized qPCR values for each miRNA by computing and outputting a predicted diagnosis using the software described herein. In some embodiments, a subject at risk of developing a motor neuron disease and/or neurodegenerative disorder is identified according to such a comparison. In some embodiments, a comparison determines that the amount of one or more miRNAs associated with neural-derived exosomes and/or circulating blood obtained from a first (or test) subject are significantly lower, or significantly higher than those obtained from a control subject.

The term “significantly” as used throughout refers to a statically significant difference that can be determined using a suitable statistical method (e.g., a t-test or Mann Whitney U-test). In some embodiments, a comparison determines that the amount of one or more miRNAs obtained from a sample of a first subject's circulating bodily fluid are significantly higher than those of a control subject, thereby indicating that the first subject has a neurogenerative disease or has a high statistical likelihood of developing a neurogenerative disease.

In some embodiments, a comparison determines that the gene fold expression amount of one or more miRNAs obtained from a sample of a subject's circulating bodily fluid is from about 1.1-fold to about 50 fold higher or lower than a baseline amount of such one or more miRNAs from a healthy subject, thereby indicating that the subject has a motor neuron disease and/or neurogenerative disorder or has a statistical likelihood of developing one (i.e., is “at risk” of developing any motor neuron disease and/or neurodegenerative disorder in general, or e.g., a motor neuron disease and/or neurodegenerative disorder such as, e.g., ALS, PLS, AD, MCI, PD, MS, HD, CJD, LBD, MSA, FTD, and/or PSP). In some embodiments, a comparison determines that the gene fold expression amount of one or more miRNAs obtained from a sample of a first subject's circulating bodily fluid is from about 1.5 to about 50-fold higher or lower, or any value or ranges of values therebetween, than the baseline amount of such one or more miRNAs, thereby indicating that the subject has a motor neuron disease and/or neurogenerative disorder or has a statistical likelihood of developing one (i.e., is “at risk” of developing any motor neuron disease and/or neurodegenerative disorder in general, or e.g., a motor neuron disease such as, e.g., ALS, PLS, AD, MCI, PD, MS, HD, CJD, LBD, MSA, FTD, and/or PSP). Throughout this description, the disclosure of range, such as from about 5 to about 10, includes any value between 5 and 10, as well as any range of values between 5 and 10 such as 5-9, or 6-10, or 6-9, or 5-8, or 5, 6, 7, 8, 9, or 10, etc.

For ALS and/or PLS, the inventors previously discovered that an amount of miR-146a-5p that is at least 1.4, at least 1.5, at least 1.6, at least 1.7, at least 1.8, or at least 1.9-fold higher than a base line amount of miR-146a-5p in circulating body fluid obtained from a subject means that the subject has or is at risk of developing ALS and/or PLS. For ALS and/or PLS, the inventors previously discovered that an amount of miR-199a-3p that is at least 1.4, at least 1.5, at least 1.6, at least 1.9, at least 2.0, or at least 2.1-fold higher than a base-line amount of miR-199a-3p in circulating body fluid obtained from a subject means that subject has or is at risk of developing ALS and/or PLS.

For ALS and/or PLS, the inventors previously discovered that an amount of miR-4454 that is at least 1.9, at least 2.1, at least 2.3, at least 2.6, or at least 2.8-fold lower that a base line amount of miR-4454 in circulating body fluid obtained from a subject means that subject has or is at risk of developing ALS and/or PLS. For ALS and/or PLS, the inventors previously discovered that an amount of miR-10b-5p that is at least 6, at least 7, at least 8, or at least 10-fold lower than a base line amount of miR-10b-5p in circulating body fluid obtained from a subject means that the subject has or is at risk of developing ALS and/or PLS. The embodiments encompass the combined application of the presence, the absence, and/or the amount of miRNA found within two more miRNA that contribute to the ability to differentiate the different disease states. The embodiments further include the selection of which miRNA, when combined, diagnose the disease state.

For ALS and/or PLS, the inventors previously discovered that an amount of miR-29b-3p that is at least 1.0, at least 1.1, at least 1.2, or at least 1.3-fold lower that a base line amount of miR-29b-3p in circulating body fluid obtained from a subject means that the subject has or is at risk of developing ALS and/or PLS. For ALS and/or PLS, the inventors previously discovered that an amount of miR-151a-3p that is at least 1.4, at least 1.6, at least 1.8, at least 1.9, at least 2.1, or at least 2.5-fold higher that a base line amount of miR-151a-3p in circulating body fluid obtained from a subject means that the subject has or is at risk of developing ALS and/or PLS.

For ALS and/or PLS, the inventors previously discovered that an amount of miR-151a-5p that is at least 1.4, at least 1.5, at least 1.6, at least 1.8, or at least 1.9-fold higher than a base-line amount of miR-151a-5p in circulating body fluid obtained from a subject means that the subject has or is at risk of developing ALS and/or PLS. For ALS and/or PLS, the inventors previously discovered that an amount of miR-199a-5p that is at least 2.0, at least 2.3, at least 2.5, at least 2.6, or at least 2.8-fold higher than a base-line amount of miR-199a-5p in circulating body fluid obtained from a subject means that the subject has or is at risk of developing ALS and/or PLS.

For AD, the inventors discovered that an amount of, for example miR-151a-3p that is at least 1.4, at least 1.5, at least 1.6, at least 1.7, at least 1.8, or at least 1.9-fold higher than a base-line amount of miR-151a-3p in circulating body fluid obtained from a healthy subject means that the subject has or is at risk of developing AD. In some embodiments, an amount of miR-151a-5p that is at least 0.9, at least 1.0, at least 1.1, or at least 1.2-fold higher than a base line amount of miR-151a-5p in circulating body fluid obtained from a healthy subject means that subject has or is at risk of developing AD. In some embodiments, an amount of miR-146a-5p that is at least 1.2, at least 1.3, at least 1.4, or at least 1.45-fold higher than a base-line amount of miR-146a-5p in circulating body fluid obtained from a healthy subject means that subject has or is at risk of developing AD.

In some embodiments, an amount of miR-4454 that is at least 2.0, at least 2.1, at least 2.2, or at least 2.3-fold lower than a base-line amount of miR-4454 in in circulating body fluid obtained from a healthy subject means that subject has or is at risk of developing AD. In some embodiments, an amount of miR-10b-5p that is at least 2.9, at least 3.0, at least 3.1, or at least 3.3-fold lower than a base-line amount of miR-10b-5p in circulating body fluid obtained from a healthy subject means that subject has or is at risk of developing AD. In some embodiments, an amount of miR-199a-3p that is at least 1.3, at least 1.4, at least 1.5, or at least 1.6-fold higher than a base-line amount of miR-199a-3p in circulating body fluid obtained from a healthy subject means that subject has or is at risk of developing AD.

In some embodiments, an amount of miR-199a-5p that is at least 1.3, at least 1.4, at least 1.5, or at least 1.6-fold higher than a base-line amount of miR-199a-5p in circulating body fluid obtained from a healthy subject means that subject has or is at risk of developing AD. In some embodiments, an amount of miR-29b-3p that is at least 1.0 higher than, or at least 1.6, at least 1.8, or at least 2.0-fold lower than a base-line amount of miR-29b-35p in circulating body fluid obtained from a healthy subject means that subject has or is at risk of developing AD. In some embodiments, an amount of miR-126-5p that is at least 1.0, at least 1.05, at least 1.15, or at least 1.2-fold lower than a base-line amount of miR-126-5p in circulating body fluid obtained from a healthy subject means that subject has or is at risk of developing AD. In some embodiments, an amount of miR-150-5p that is at least 3.0, at least 3.3, at least 3.5, or at least 3.8-fold lower than a base-line amount of miR-150-5p in circulating body fluid obtained from a healthy subject means that subject has or is at risk of developing AD.

In some embodiments, an amount of, for example miR-151a-3p that is at least 1.4, at least 1.5, at least 1.6, at least 1.7, at least 1.8, or at least 1.9-fold higher than a base-line amount of miR-151a-3p in circulating body fluid obtained from a healthy subject means that the subject has or is at risk of developing MCI. In some embodiments, an amount of miR-151a-5p that is at least 0.9, at least 1.2, at least 1.3, or at least 1.7-fold higher than a base-line amount of miR-151a-5p in circulating body fluid obtained from a healthy subject means that subject has or is at risk of developing MCI. In some embodiments, an amount of miR-146a-5p that is at least 1.3, at least 1.4, at least 1.5, or at least 1.6-fold higher than a base-line amount of miR-146a-5p in circulating body fluid obtained from a healthy subject means that subject has or is at risk of developing MCI.

In some embodiments, an amount of miR-4454 that is at least 7.0, at least 7.1, at least 7.2, or at least 7.4-fold lower than a base-line amount of miR-4454 in circulating body fluid obtained from a healthy subject means that subject has or is at risk of developing MCI. In some embodiments, an amount of miR-10b-5p that is at least 4.5, at least 4.7, at least 4.9, or at least 5.0-fold lower than a base-line amount of miR-10b-5p in circulating body fluid obtained from a healthy subject means that subject has or is at risk of developing MCI. In some embodiments, an amount of miR-199a-3p that is at least 1.4, at least 1.5, at least 1.6, or at least 1.7-fold higher than a base-line amount of miR-199a-3p in circulating body fluid obtained from a healthy subject means that subject has or is at risk of developing MCI.

In some embodiments, an amount of miR-199a-5p that is at least 1.7, at least 1.8, at least 2.0, or at least 2.2-old higher than a base-line amount of miR-199a-5p in circulating body fluid obtained from a healthy subject means that subject has or is at risk of developing MCI. In some embodiments, an amount of miR-29b-3p that is at least 1.0, at least 1.1, at least 1.2, or at least 1.3-fold lower than a base-line amount of miR-29b-3p in circulating body fluid obtained from a healthy subject means that subject has or is at risk of developing MCI. In some embodiments, an amount of miR-126-5p that is at least 1.0, at least 1.1, at least 1.2, or at least 1.25-fold lower than a base-line amount of miR-126-5p in circulating body fluid obtained from a healthy subject means that subject has or is at risk of developing MCI. In some embodiments, an amount of miR-150-5p that is at least 5.7, at least 5.8, at least 5.9, or at least 6.0-fold lower than a base-line amount of miR-150-5p in circulating body fluid obtained from a healthy subject means that subject has or is at risk of developing MCI.

In some embodiments, such a comparison determines that the gene fold expression amount of one or more of the miRNAs listed in the final miRNA panel that was identified in circulating bodily fluid obtained from a subject, is from about 1.1-fold to about 75-fold higher or lower than the baseline amount of such one or more miRNAs from a healthy subject, thereby indicating that the subject has a motor neuron disease and/or neurodegenerative disorder, or has a statistical likelihood of developing one (i.e., is “at risk” of developing any motor neuron disease and/or neurodegenerative disorder in general, or e.g., a motor neuron disease such as, e.g., ALS, PLS, AD, MCI, PD, MS, HD, CJD, LBD, MSA, FTD, and/or PSP). Using the guidelines provided herein, as well as the examples provided below, a person having ordinary skill in the art will be capable of repeating the same or similar experimentation for each of the motor neuron diseases and/or neurodegenerative disorders listed in Table 1 above, as well as additional diseases and/or disorders, and developing a suitable miRNA list for each motor neuron diseases and/or neurodegenerative disorders that can be used to diagnose the specific disease and/or disorder and to distinguish it from other diseases and/or disorders listed in the final miRNA panel. Thus, the full miRNA panel can be completed and supplemented from time to time.

The term “base-line amount” as used herein refers to an average, mean, or absolute amount of one or more miRNAs obtained from circulating bodily fluid obtained from one or more suitable control subjects. For example, a control subject can be a subject that does not have a motor neuron disease and/or neurodegenerative disorder. In one embodiment, a healthy subject is a young adult (e.g., within the ages of 18-30) that shows no signs or symptoms of a motor neuron disease and/or have no family history of a motor neuron disease. In another embodiment, a healthy subject is an older adult (e.g., within the ages of 45-100) that shows no signs or symptoms of a motor neuron disease and/or has no family history of a motor neuron disease.

In some embodiments, the presence or absence of a motor neuron disease and/or neurodegenerative disorder in a subject is determined according to an amount of one or more miRNAs that is associated with circulating bodily fluid obtained from a subject. In some embodiments, an amount of at least 0.01 mean normalized copy number/μl of miRNA, at least 0.05 mean normalized copy number/μl of miRNA, at least 0.1 mean normalized copy number/μl of miRNA, at least 0.5 mean normalized copy number/μl of miRNA, at least 1 mean normalized copy number/μl of miRNA, at least 3 mean normalized copy number/μl of miRNA, at least 5 mean normalized copy number/μl of miRNA, at least 10 mean normalized copy number/μl of miRNA, or more of miRNA that is associated with circulating bodily fluid obtained from a subject indicates that the subject has a motor neuron disease or has a statistical likelihood (i.e., is “at risk”) of developing a motor neuron disease. In other embodiments, an amount of at least 50 pg/μL of miRNA, at least 100 μg/μL of miRNA, at least 150 μg/μL of miRNA, at least 200 pg/μL of miRNA, at least 250 μg/μL of miRNA, at least 300 μg/μL of miRNA, at least 350 pg/μL of miRNA, at least 400 μg/μL of miRNA, at least 450 μg/μL of miRNA, at least 500 pg/μL of miRNA, at least 550 μg/μL of miRNA, at least 600 μg/μL of miRNA, at least 650 pg/μL of miRNA, at least 700 μg/μL of miRNA, at least 750 μg/μL of miRNA, at least 800 pg/μL of miRNA, at least 850 μg/μL of miRNA, at least 900 μg/μL of miRNA, at least 950 pg/μL of miRNA, at least 1000 μg/μL of miRNA, or more of miRNA that is associated with circulating bodily fluid obtained from a subject indicates that the subject has a motor neuron disease or has a statistical likelihood (i.e., is “at risk”) of developing a motor neuron disease. In an embodiment, the one or more miRNAs are selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, or from any listed in Tables 1 and 2 above.

In some embodiments, methods are provided for monitoring the progression of a motor neuron disease and/or neurodegenerative disorder in a subject. In some embodiments, such a method comprises a) obtaining a circulating bodily fluid sample from the subject; (b) determining an amount of one or more miRNAs present in the circulating bodily fluid, such as one or more miRNAs selected from those listed in the final miRNA panel, without determining an amount from a subject's neural-derived exosomes; and (c) comparing the amount of the one or more miRNAs determined in step (b) to a baseline amount of the one or more miRNAs; and (d) determining the progression or lack of progression of the motor neuron disease and/or neurodegenerative disorder based on the comparison in (c).

In some embodiments, methods are provided for monitoring a response to treatment of a motor neuron disease and/or neurodegenerative disorder in a subject. In some embodiments, such a method comprises a) obtaining a circulating bodily fluid sample from the subject; (b) determining an amount of one or more miRNAs present in the circulating bodily fluid, such as one or more miRNAs selected from those listed in the final miRNA panel, without determining an amount from a subject's neural-derived exosomes; (c) comparing the amount of the one or more miRNAs determined in step (b) to a baseline amount of the one or more miRNAs; and (d) monitoring the response to treatment based on the comparison in (c). In some embodiments, a difference between an amount of one or more miRNAs from the circulating bodily fluid obtained from a subject after treatment of the subject has commenced and a baseline amount that is less than the difference obtained at an earlier point in time indicates that the subject has responded favorably to the treatment. In some embodiments, a difference between an amount of one or more miRNAs from the circulating bodily fluid obtained from a subject and a baseline amount that is greater than the difference obtained at an earlier point in time indicates that the subject has not responded favorably to the treatment. In embodiments discussed herein, reference to determining an amount of miRNA may denote determining the gene fold expression amount based on a geometric mean representing the normalization of raw cycle Cq numbers with three or more endogenous miRNA, the geometric mean represented by a deltaCq value.

In some embodiments, the baseline amount may be an amount considered ‘normal’ for the particular miRNA (e.g., an average amount for age-matched individuals not diagnosed with the motor neuron disease), or the baseline amount may be a historical reference amount for the particular subject (e.g., a baseline amount that was obtained from a circulating bodily fluid sample derived from the same subject, but at an earlier point in time). Quantitative baseline amounts that are determined contemporaneously (e.g., a reference value that is derived from a pool of samples including the sample being tested) are also contemplated. Accordingly, in some embodiments, methods are provided for monitoring progression of a motor neuron disease and/or neurodegenerative disorder in a subject by obtaining a quantitative measured amount for one or more miRNAs present in circulating bodily fluid obtained from a sample and comparing such measured value to a baseline amount. In some embodiments, a difference between an amount of one or more miRNAs from circulating bodily fluid obtained from a subject and a baseline amount that is less than the difference obtained at an earlier point in time indicates that the progression of the disease and/or disorder has diminished. In some embodiments, a difference between an amount of one or more miRNAs from circulating bodily fluid obtained from a subject and a baseline amount that is greater than the difference obtained at an earlier point in time indicates that the progression of the disease and/or disorder has increased.

### Administration

Any suitable method of administering a treatment or drug to a subject can be used. Any suitable formulation and/or route of administration can be used for administration of a treatment or drug disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's disease, condition, symptoms, weight, age, and/or general health. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments administering a drug to a subject comprises providing the drug to the subject, for example for self-administration or for administration to the subject by another (e.g., by a non-medical professional). As another example, a drug can be provided as an instruction written by a medical practitioner that authorizes a patient to be provided a drug or treatment described herein (e.g., a prescription). In yet another example, a drug can be provided to a subject where the subject self-administers a composition orally, intravenously or by way of an inhaler, for example. Alternately, one can administer a drug in a local rather than systemic manner, for example, via direct application to the skin, mucous membrane or region of interest for treating, including using a depot or sustained release formulation. In certain embodiments a drug is administered alone (e.g., as a single active ingredient (AI) or, e.g., as a single active pharmaceutical ingredient (API)). In other embodiments, a drug is administered in combination with one or more additional AIs/APIs, for example, as two separate compositions or as a single composition where the one or more additional AIs/APIs are mixed or formulated together with a drug in a pharmaceutical composition.

In some embodiments, an amount of a motor neuron disease and/or neurodegenerative disorder drug administered to a subject is a therapeutically effective amount. In some embodiments, a therapeutically effective amount of a drug is an amount needed to obtain an effective therapeutic outcome. In certain embodiments, a therapeutically effective amount of a drug is an amount sufficient to treat, reduce the severity of, inhibit or delay the onset of, mitigate and/or alleviate one or more symptoms of a motor neuron disease. Determination of a therapeutically effective amount is well within the capability of those skilled in the art, especially in light of the detailed disclosure provided herein.

In certain embodiments, a therapeutically effective amount is an amount high enough to provide an effective therapeutic effect (e.g., a beneficial therapeutic effect) and an amount low enough to minimize unwanted adverse reactions. Accordingly, in certain embodiments, a therapeutically effective amount of a drug may vary from subject to subject, often depending on age, weight, general health condition of a subject, severity of a condition being treated and/or a particular combination of drugs administered to a subject. Thus, in some embodiments, a therapeutically effective amount is determined empirically. Accordingly, in certain embodiments, a therapeutically effective amount of a drug that is administered to a subject can be determined by one of ordinary skill in the art based on amounts found effective in animal or clinical studies, a physician's experience, and/or suggested dose ranges or dosing guidelines.

In certain embodiments, a therapeutically effective amount of L-serine or a composition disclosed herein comprises one or more doses (administered to a subject) comprising at least 0.1 mg/kg, at least 5 mg/kg, at least 10 mg/kg, at least 15 mg/kg, at least 20 mg/kg, at least 25 mg/kg, at least 50 mg/kg, at least 100 mg/kg, at least 250 mg/kg, at least 500 mg/kg, at least 1000 mg/kg, at least 5000 mg/kg, or at least 7500 mg/kg of L-serine, or a salt, a precursor, derivative or conjugate thereof, per kg body weight of a subject.

In some embodiments administering a therapeutically effective amount of a motor neuron disease and/or neurodegenerative disorder drug or composition disclosed herein comprises administering a suitable dose hourly, every 2 hours, every 4 hours, every 6 hours, every 8 hours, or every 12 hours. In certain embodiments, a motor neuron disease and/or neurodegenerative disorder drug can be administered at least one, at least two, at least three, at least four, at least five, or at least six times per day, e.g., 1 to 12 times per day, 1 to 8 times per day, or 1 to 4 times per day per day. In certain embodiments, a motor neuron disease drug disclosed herein can be administered once, twice, 3 times, 4 times, 5 times, 6 times, 7 times, 8 times, 9 times, 10 times, 11 times, or 12 times per day. A motor neuron disease and/or neurodegenerative disorder drug may be administered in a single dosage form or one or more dosage forms. A daily dose can be achieved in the form of a single dose or in the form of a plurality of partial doses.

A motor neuron disease and/or neurodegenerative disorder drug disclosed herein can be administered on a daily basis or on a schedule containing days where dosing does not take place. For example, dosing may take place every other day, or dosing may take place for 2, 3, 4, or 5 consecutive days of a week, then be followed by from 1 to 5 non-dosing days.

A motor neuron disease and/or neurodegenerative disorder drug can be administered for at least a day, at least two days, at least three days, at least four days, at least five days, at least a week, at least two weeks, at least three weeks, at least a month, at least two months, at least three months, at least six months, at least a year, at least two years, or more, or for any extended duration to further improve, maintain, or retain therapeutic efficacy. In certain embodiments, a motor neuron disease and/or neurodegenerative disorder drug is administered for a duration of 1 week to 10 years or more. In some embodiments administering a therapeutically effective amount of a drug, or a pharmaceutical composition comprising a drug, comprises administering a suitable dose at a frequency or interval as needed to obtain an effective therapeutic outcome. In some embodiments administering a therapeutically effective amount of a drug or a pharmaceutical composition disclosed herein comprises administering a suitable dose hourly, every 2 hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. In some embodiments, a therapeutically effective amount of a drug or a pharmaceutical composition comprising a therapeutically effective amount of drug is administered continuously by, for example by intravenous administration.

### Kits

In some aspects, presented herein is one or more kits useful in carrying out a method of identifying a subject who has, or is at risk of developing any, or an identified motor neuron disease and/or neurodegenerative disorder that includes one or more multi-well plates, with each well containing one or more miRNA primers targeting the miRNA selected from those listed in the final miRNA panel, and a mechanism to calculate the likelihood of a positive or negative diagnosis, and/or probability of having or not having any, or an identified motor neuron disease and/or neurodegenerative disorder. In one embodiment, the kit includes two well-plates in which one or both plates include wells containing one or more miRNA primers targeting the miRNA selected from the list above, and Quality Control (QC)/spike-in/calibration controls in which one or more wells contain QC primers, no-template controls, and/or spike-in primers designed to monitor the success of the method. In another embodiment, the kit includes one well-plate in which the wells include one or more miRNA primers targeting the miRNA selected from the list above, QC primers, no-template controls, and/or spike-in primers designed to monitor the success of the method. In a further embodiment, the mechanism to calculate the likelihood of a positive or negative diagnosis, and/or probability of having or not having a motor neuron disease and/or neurodegenerative disorder includes software configured to carry out calculations using random forest machine learning algorithms and/or logistic regression algorithms, and/or other algorithms developed for classification of known disease and healthy control plasma samples.

The calculation may be performed using random forest machine learning algorithms and/or logistic regression algorithms developed from machine learning classification of known disease and healthy control plasma samples. Random forest machine learning algorithms are well-known and described in the art, and typically rely on random forest algorithms that have three main hyperparameters: (i) node size; (ii) number of trees; and (iii) number of features sampled, that are predetermined. Logistic regression machine learning algorithms also are well known in the art and are used to accomplish binary classification tasks by predicting the probability of an outcome, event, or observation. Using the techniques described herein, those skilled in the art will be capable of using random forest machine learning algorithms and/or logistic regression algorithms to develop software or a protocol for carrying out the method of identifying a subject who has, or is at risk of developing a motor neuron disease and/or neurodegenerative disorder using the values obtained from sampling and quantification of the gene fold expression, and normalization thereof, of two or more, three or more, four or more, five or more, six or more, seven or more, or up to all of the miRNA in each column of Table 1 above (i.e., the miRNA listed for each disease or disorder in Table 1). The software can be provided with one or more kit(s) described herein the first time the purchaser acquires a kit, and loaded onto a computing device integral to, incorporated into, or in communication with a device for conducting qPCR.

The well plates useful in the kits described herein may include, for example, qPCR plates of varying sizes, including, but not exclusively, 48, 96, and 384 well plates. Each of the respective wells can pre-loaded with one or more of miRNA primers targeting the miRNA selected from those listed in the final miRNA panel, as well as QC primers, no-template controls, and/or spike-ins. Particularly preferred QC primers, no-template controls, and/or spike-in primers that may be used in the kit(s) described herein are described in more detail in the examples below. In one embodiment, a kit may contain one or two 96-well plates suitable for 10 μL or 25 μL reactions in which one of the well-plates contains wells spiked/spotted with one or more of the miRNA primers targeting one or more of the miRNAs selected from those listed in the final miRNA panel, and the other well-plates contains wells filed with QC primers, no-template controls, and/or spike-in primers. In another embodiment, a kit may contain one 96-well plates suitable for 10 μL or 25 μL reactions in which the well-plates are filled with one or more of the miRNA primers detailed above. In another embodiment, the kit may contain a 384-well plate suitable for 10 μL reactions, in which all of the miRNA, QC primers, no-template controls, and/or spike-in primers are included on the same plate or one 384-well plate with only primers from above, or two plates in which all of the miRNA, QC primers, no-template controls, and/or spike-in primers are included across the two plates.

The kits are particularly suited for automated pipetting using a liquid handling robot (e.g., an Opentrons OT-2 robot) for sample loading into the respective wells in the plate(s) of the respective kit(s) that are preloaded with the reagents discussed above. Operators supply the RNA/cDNA. The kits can be designed for use in any analyzer capable of carrying out qPCR analysis on the circulating bodily fluid samples added to the respective well-plates, and indicating a presence and/or amount of one or more of the miRNAs selected from those listed in the final miRNA panel. The method of diagnosis can be carried out using the information generated regarding the presence and/or amount of one or more of the miRNAs selected from those listed in the final miRNA panel.

The diagnostic method preferably is carried out on-site using a mechanism for calculating the likelihood of a positive or negative diagnosis, and/or probability of having or not having a motor neuron disease and/or neurological disorder. A preferred device for conducting the qPCR includes the use of a conventional real-time PCR (qPCR) machine, such as those available as BioRad CFX series, Applied Biosystems, Edvotek, MSE PRO series, Benchmark Scientific, Roche, Thermo Scientific and the like. The data acquired from the qPCR machine can be fed to a computing apparatus configured to calculate the likelihood of a positive or negative diagnosis, and/or probability of having or not having a motor neuron disease and/or neurological disorder.

The calculation may be performed using random forest machine learning algorithms, logistic regression algorithms (e.g., cross-validation models, XGBoost, Lasso, glm, glm-net, etc.), and/or other classification algorithms (eg. K-Nearest Neighbors, Decision Tree, Support Vector Machine (SVM), Naïve Bayes, K-means Clustering, Hierarchical Clustering, Gaussian Mixture Models (GMMs), Pseudo-Labeling, or Co-Training) developed from machine learning classification of known disease and healthy control plasma samples, or plasma from other disease classes. Using the guidelines provided herein, those skilled in the art are capable of using random forest machine learning algorithms and/or logistic regression algorithms to develop software or a protocol for carrying out the method of identifying a subject who has, or is at risk of developing a motor neuron disease and/or neurodegenerative disorder using the discoveries described herein and using the presence or absence of two or more, three or more, four or more, five or more, six or more, seven or more, or eight or more of miRNA selected from those listed in the final miRNA panel. The software can be provided with one or more kit(s) described herein the first time the purchaser acquires a kit, and loaded onto a computing device integral to, incorporated into, or in communication with a device for conducting qPCR.

### The Model

Certain embodiments also relate to a model and system useful in detecting the presence or absence of one or more motor neuron diseases and/or neurodegenerative disorders. The model can be developed using samples obtained from healthy individuals and from individuals known to have a specific motor neuron disease and/or neurodegenerative disorder such as but not limited to ALS, PLS, AD, MCI, PD, MS, HD, CJD, LBD, MSA, FTD, and PSP, in which the samples are processed to extract RNA, synthesize cDNA, amplify miRNA using, for example, qPCR, and assessing the differences in expression of various miRNA between healthy individuals and those known to have a specific motor neuron disease and/or neurodegenerative disorder. The model can be created by comparing miRNA from diseased subjects to healthy control subjects, assessing the fold differences between each miRNA from diseased subjects and healthy control subjects, and generating a list of miRNAs that exhibited sufficiently consistent fold changes between diseased subjects and healthy controls. Sufficiently consistent fold changes denote a fold change of at least 1.0 between either diseased subjects and healthy controls, as well as whether the miRNA were upregulated or downregulated. For example, if miR-151a-3p was consistently upregulated with a fold change of more than 1.5 in AD subjects when compared to healthy controls, then miR-151a-3p would be included in the AD list in the miRNA panel. Similarly, if miR-4454 was consistently downregulated by more than 2.0 in AD subjects when compared to healthy controls, then miR-4454 would be included in the AD list in the miRNA panel.

Using the miRNA identified above as having sufficiently consistent fold changes, the model can be further developed by quantifying the abundance of each of the miRNAs identified in the model using qPCR techniques. The raw cycle quantitation (Cq) numbers can then be normalized by the geomean of three or more endogenous miRNA (generally the more stably expressed miRNAs, one or more of which may not be among the miRNA listed in the panel), thus generating a delta Cq value for each miRNA. These deltaCq values may then be used in a logistic regression analysis, using a Lasso selection feature to determine which of the miRNA from the list above contributed the most to separating a specific motor neuron disease and/or neurodegenerative disorder subject from healthy controls, and then assigning a coefficient to each of the selected miRNA based on the relative contribution of each miRNA in separating a specific motor neuron disease and/or neurodegenerative disorder subject from healthy control subjects. Once the miRNAs are selected, the final diagnostic/prognostic test may be run without the Lasso feature selection. The logistic regression analysis may be carried out by conducting a relaxed fit, no penalty, multinomial logistic regression (glmnet) in R-studio with four libraries: data.table, glmnet, knitr, and caret. The regression used a lasso feature selection which kept out-of-fold predictions and included the following parameters: seed set of 42, multinomial family, grouped type, mse measure, Z-score standardization, intercept included, lambda sequence (exp(seq(−4, −1, length.out=1000), 20 nfolds, convergence threshold of 1 e−6, 1 e7 maximum iterations. The analysis creates a select list of miRNAs that contributed the most to distinguishing between disease and healthy subject, and assigned a coefficient to each miRNA.

Using this model, the combined qPCR data from a single individual can then be compared to the model to determine a disease classification prediction for that individual. Specifically, the raw cycle quantitation (Cq) numbers for the list of miRNAs developed in the model can then be normalized by the geomean of three or more endogenous miRNA chosen for their stability within the sample matrix, thus generating a delta Cq value for each miRNA, using the techniques discussed above and in the examples below. These delta Cq values can then be compared to the logistic regression model developed from the database to determine the prediction of disease state incrementally using the value of each miRNA. In the multinomial model, a positive coefficient means that a one-unit increase in the value for that specific miRNA increases the log-odds of the individual having disease A, (e.g., AD or ALS), and a negative coefficient decreases the log-odds of the person having disease B (e.g., PD or PLS) and C (healthy), relative to the overall average. When the coefficient is negative, a one-unit increase in the value for that specific miRNA decreases the log-odds of the individual having disease A, while holding other variables constant. The combination of log-odds for each miRNA in the model (e.g., the equation of the log-odds calculated which includes the coefficients) determines the final prediction that a person does or does not have the disease or disorder. The model adds up the likelihood that the person has A (e.g., AD or ALS) by adding up each of the log odds ratios calculated from the coefficients assigned to each miRNA in the model. It then generates a prediction number that indicates that the person is predicted to have the disease with a certain percentage certainty, (e.g., person has AD with 95% certainty).

The model of the embodiments therefore includes at least: (1) a database stored in a storage medium, wherein the database comprises delta Cq values for individuals with a known disease status, a list of miRNA for each disease or disorder present in a final miRNA panel, and a coefficient associated with each miRNA, wherein the list of miRNA is selected from 4 or more miRNA selected from the specific column in the final miRNA panel (e.g., Table 1) that corresponds to the disease. The respective coefficients are used to calculate the log-odds that the subject has a given disease state (or does not have a certain disease state); and (2) software configured to access the database, process qPCR generated quantification data for each miRNA in the final miRNA panel; generate normalized qPCR values for each miRNA in the final miRNA panel, and process the normalized qPCR values for each miRNA by computing and outputting a predicted diagnosis of the presence or absence of a motor neuron disease and/or neurodegenerative disorder using the software. The predictive certainty uses parameters pre-set into the software to provide the computation.

In one embodiment, the database of the model includes a list of miRNA useful in distinguishing between various motor neuron diseases or neurodegenerative disorders and healthy subjects. The list of miRNAs can be in the form of an miRNA panel that includes columns (or sets) indicative a specific motor neuron disease or neurodegenerative disorder listing the miRNA relevant to that specific motor neuron disease or neurodegenerative disorder. An exemplary list of miRNAs is shown in Table 1 above. The database also will include a coefficient associated with miRNA in the miRNA panel.

Another embodiment includes a method of creating a model suitable for use in diagnosing the presence or absence of one or more motor neuron diseases and/or neurodegenerative disorders. The method includes a method of generating a database comprising a set of miRNA and coefficients corresponding to each miRNA in the set, and creating software configured to process miRNA data from a sample from a subject and generate a diagnosis of whether the subject has, is susceptible to having, or does not have a specific motor neuron disease and/or neurodegenerative disorder. The method of generating the database may include obtaining samples from subjects known to have a motor neuron disease and/or neurodegenerative disorder, and subjects known to be healthy, extracting miRNA from exosomes from the respective samples, synthesizing cDNA for each miRNA, amplifying the cDNA using, for example, qPCR to determine the quantification cycle (Cq) values for each miRNA. The database may be further created by normalizing the Cq values for each of miRNA using the geometric mean of three or more stable reference miRNAs, and determining the gene-fold expression of each miRNA from the delta Cq values.

The database can further be created by assessing the differences in expression of the extracted miRNA between healthy individuals and those known to have a specific motor neuron disease and/or neurodegenerative disorder. The database can be created by comparing miRNA from specific motor neuron disease and/or neurodegenerative disorder subjects (“diseased subjects”) to healthy control subjects, assessing the fold differences between each miRNA from diseased subjects and healthy control subjects, and generating an initial list of miRNA that exhibited sufficiently consistent fold changes between diseased subjects and healthy controls. Sufficiently consistent fold changes denote a fold change of at least 1.0 between either AD or MCI and healthy controls, as well as whether the miRNA were upregulated or downregulated.

The database can be further created by conducting a logistic regression analysis on the initial list of miRNA by subjecting the delta Cq values for each of the miRNA in the initial list to an iterative regression analysis using a Lasso selection feature by comparing delta Cq values of miRNA from known diseased subjects to delta Cq values of miRNA from known healthy controls. The iterative regression analysis with the Lasso selection feature can determine which miRNA contribute the most to a specific disease or disorder, and which miRNA contribute the most to healthy controls, and will generate a second, smaller list of miRNA from the above initial list, along with a coefficient associated with each miRNA in the second list that represents the relative contribution of each miRNA in separating diseased subjects from one another, and from healthy control subjects.

The iterative regression analysis with the Lasso selection feature ends when the predictive accuracy of disease or disorder, or healthy is at or above about 80%, thereby generating a database that includes a second list of miRNA and coefficients corresponding to each miRNA. The completion of the regression analysis also establishes the parameters in the software for carrying out subsequent regression analyses on samples from unknown subjects, thereby creating the software configured to process miRNA data from a sample from a subject and generate a diagnosis of whether the subject has, is susceptible to having, or does not have a motor neuron disease and/or neurodegenerative disorder. The model can be continuously updated by repeating the above method with additional samples from subjects known to have a specific motor neuron disease and/or neurodegenerative disorder, or from healthy controls, where the updated model may create minor modifications to the miRNA in the second list along with their respective coefficients, and modify the parameters in the software.

The embodiments also include a method of using the model to diagnose a subject as either having or being susceptible of developing a specific motor neuron disease and/or neurodegenerative disorder, or as not having or being susceptible of developing a specific motor neuron disease and/or neurodegenerative disorder. The method includes obtaining a sample from a subject, extracting miRNA from the sample, synthesizing cDNA for each miRNA listed in the model (e.g., those miRNA from the second list), amplifying the cDNA using, for example, qPCR and determining the quantification cycle (Cq) values for each miRNA. The raw cycle quantitation (Cq) numbers for the miRNA developed in the model can then be normalized by the geomean of three or more endogenous miRNA, thus generating a delta Cq value for each miRNA, using the techniques discussed above. These delta Cq values may then be input into the software created in developing the model in which they are subjected to a mathematical comparison to the logistic regression analysis with its predetermined parameters generated in developing the model.

The method may then determine how each miRNA value contributes to the prediction of disease state incrementally and will generate a coefficient for each miRNA that is indicative of the log-odds of the subject having or being susceptible of developing a specific motor neuron disease and/or neurodegenerative disorder, or as not having or being susceptible of developing a specific motor neuron disease and/or neurodegenerative disorder. The method may further include combining the log-odds for each miRNA in the model to generate the final prediction that a person does or does not have, or is not susceptible to having a specific motor neuron disease and/or neurodegenerative disorder, in accordance with the following equation 1 below, a linear regression model used in predictive modeling. For example, the software may add up each of the log odds ratios using the coefficients assigned to each miRNA in the model, and then generate a prediction number that indicates that the person is predicted to have the disease with a certain percentage certainty, (e.g., person has AD). with 95% certainty).

$$\begin{matrix}{p}_{j} ( x ) := ℙ [ Y = j \vert {X}_{1} = {x}_{1} , … , {X}_{p} = {x}_{p} ] \\ = \frac{{e}^{{β}_{\text{?}} + {β}_{\text{?}} {X}_{\text{?}} + … + {β}_{\text{?}} {X}_{\text{?}}}}{1 + {\sum }_{ℓ = 1}^{J - 1} {e}^{{β}_{\text{?}} + {β}_{\text{?}} {X}_{\text{?}} + … + {β}_{\text{?}} {X}_{\text{?}}}}\end{matrix} \qquad Equation 1$$

$$\text{?} \text{indicates text missing or illegible when filed}$$

In Equation 1, pj(x) is the prediction of disease state, Y represents categorical data variables, {1, . . . ,J} represents the disease groups, and X1, . . . . Xp represents the miRNA predictors. Information regarding predictive modeling is available, for example, at egarpor.github.io/PM-UC3M, incorporated herein by reference in its entirety.

### EXAMPLES

The Examples of U.S. Patent Application Publication No. 2021/0164051 are incorporated herein by reference in their entirety. The Examples of this earlier application provide detailed information regarding the use of neural derived exosomes to determine the presence or amount of one or more, or combinations of certain miRNA associated with the neural derived exosomes. These Examples provide additional information regarding the determination of whether a distinct difference in the presence and/or amounts of one or more, or combinations of certain miRNA associated with the neural derived exosomes is found between healthy, or control, subjects, and subjects known to have a motor neuron disease. The examples below show how the method can be carried out to determine a suite of miRNAs for ALS, PLS, AD, and MCI. These examples can be repeated for additional motor neuron diseases and/or neurodegenerative disorders such as MS, HD, PD, CJD, PSP, and others.

### Example 1—an miRNA Fingerprint of miRNA for Amyotrophic Lateral Sclerosis/Motor Neuron Disease Using Neural-Derived Exosomes

Using retrospective blood samples from previously identified ALS, primary lateral sclerosis, Parkinson's disease patients, and healthy controls, an eight-miRNA fingerprint was evaluated as an aid to diagnosing ALS and/or PLS.

Blood plasma from 119 ALS, 42 primary lateral sclerosis, 20 Parkinson's disease patients, and 150 healthy controls were evaluated for miRNAs contained within neural enriched extracellular vesicles. Primary lateral sclerosis is considered an ALS-mimic and Parkinson's disease was also included for comparison as a neurological control. Ghasemi, “M. Amyotrophic lateral sclerosis mimic syndromes,” Iran J Neurol. 15(2):85-91 (2016). The sample number was limited by the availability of samples. Sample inclusion eligibility was based on diagnosis for ALS, primary lateral sclerosis, or Parkinson's disease, and the absence of a neurological diagnosis for healthy control samples.

100 ALS blood samples, collected across the USA between 2017-2018, were accessed through the USA National ALS Biorepository maintained by the Centers for Disease Control and Prevention, and the Agency for Toxic Substances and Disease Registry (CDC, Advarra IRB Pro00053269). Nineteen ALS blood samples came from a Phase IIa clinical trial (NCT03580616, Dartmouth Institutional Review Board D18095, collected between 2019-2022). 42 blood plasma samples were used from the multi-site primary lateral sclerosis natural history study by Dr. Hiroshi Mitsumoto (Approved by the Columbia University Institutional Review Board (IRB), and by the IRB at each individual site; samples were collected in 2021-2023 following the protocols set forth in Mitsumoto et al., “Primary lateral sclerosis natural history study-planning, designing, and early enrollment,” Amyotrophic Lateral Sclerosis and Frontotemporal Degeneration, 24(5-6):394-404 (2023)).

Primary lateral sclerosis was characterized clinically by pure upper motor neuron dysfunction, however, the time since symptom onset is widely accepted by investigators as part of the diagnostic criteria. Samples used in this study fell into three categories: (1) “early PLS” was defined as having upper motor neuron signs present for less than two years after symptom onset (n=4); (2) “probable PLS” was defined as having symptom onset between two to four years (n=18); and (3) patients with a disease duration longer than 4 years since symptom onset were defined as “definite PLS” (n=19). However, no patients were admitted with a disease duration more than 15 years.

20 Parkinson's disease blood plasma samples were obtained from Precision for Medicine (Bethesda, MD), collected across the USA between 2018 and 2020 (Norton, M A, CR00425931). The 150 controls, without known neurological disease, were sourced from Innovative Research Inc (Novi, M I, FDA Approval, #3003372368, collected across the USA prior to 2022) and Precision for Medicine (Norton, M A, CR00425931, collected across the USA from 2020-2022) and were considered to be healthy controls. Selection of these healthy controls were chosen to match the gender and age characteristics of the CDC ALS cohort where possible. Informed consent from all participants was obtained through the respective institutions involved. Since this study used only de-identified participant data, Advarra IRB (Pro00053269) determined that it “does not meet the DHHS definition of human subjects research under 45 CFR 46 and, therefore, does not require IRB oversight.” During the laboratory analyses, researchers were blinded as to the status of the samples and whether they represented disease or control patients.

Blood samples were collected in K2 EDTA tubes and because the samples were obtained from different institutions, plasma processing protocols varied. Samples were blinded before being processed and analyzed. The following methods were used to analyze the samples.

### Extracellular Vesicle Isolation

Extracellular Vesicles (EV) were processed as reported in prior studies using polymer-based precipitation and immunoassay purification: Banack S A, et al., “An miRNA fingerprint using neural-enriched extracellular vesicles from blood plasma: Towards a biomarker for amyotrophic lateral sclerosis/motor neuron disease,” Open Biology, 10(6):200116 (2020); and Banack S A, et al., “miRNA extracted from extracellular vesicles is a robust biomarker of amyotrophic lateral sclerosis,” J. Neurol Sci., 442:120396 (2022). Characterization of EVs and differences between total EV collection, neural enriched EV, and the total minus neural enriched extracellular vesicles fraction has been previously published: Banack (2020); and Dunlop R A, et al., “L1CAM immunocapture generates a unique extracellular vesicle population with a reproducible miRNA fingerprint,” RNA biology, 20(1):140-148 (2023).

### RNA Extraction

RNA extraction was conducted according to the instructions in Qiagen RNeasy Midi Kit Part 2: RNA isolation. We added spike-ins for monitoring RNA extraction efficiency (UniSp2, UniSp4, UniSp5, available as part of the RNA Spike-in Kit, For RT, (Qiagen, cat. no. 339390) at 1 μL per 700 μL lysis buffer (See Dunlop et al., “A comparison of the efficiency of RNA extraction from extracellular vesicles using the Qiagen RNeasy MinElute versus Enzymax LLC RNA Tini Spin columns and qPCR of miRNA,” Biol Methods Protoc., 6(1):1-9 (2021) for a detailed modified protocol).

cDNA Synthesis

cDNA was synthesized using the miRCURY LNA RT Kit (Qiagen, cat. no. 339340) according to the manufacturer's instructions, and as described previously in Dunlop (2021) above. Modifications included the addition of 1 μL of a spike-in control mix containing UniSp6 and Caenorhabditis elegans cel-miR-39-3p to each cDNA reaction to monitor cDNA synthesis (reverse transcription) efficiency. UniSp6 is provided in the miRCURY LNA RT kit, and cel-miR-39-3p is included in the RNA Spike-in Kit, For RT. 4 μL of total RNA was used rather than 2 μL as recommended by the manufacturer, since it was previously determined that 4 μL returns a more robust signal. Each 10 μL cDNA synthesis reaction was conducted in duplicate and pooled to create a total of 20 μL cDNA. Prior to storage at −20 °C, cDNA was aliquoted into 3 μL aliquots to avoid multiple freeze/thaw cycles.

Real-Time Quantitative PCR (qPCR) Quality Control

Following cDNA synthesis, quality control (QC) qPCR was carried out for every sample on eleven QC miRNA targets. To determine RNA extraction efficiency, the Cqs for the spike-ins UniSp2, UniSp4, and UniSp5 were measured. To determine if reverse transcription (RT) (cDNA synthesis) proceeded without inhibition, the spike-ins UniSp6 and cel-miR-39-3p were measured. Sample signal is a measure of the amount of cDNA in each sample and determines whether samples are suitable for downstream analysis. For determination of sample signal, five endogenous miRNAs: miR-142-3p, miR-451a, miR-23a-3p, miR-30c-5p, and miR-103a-3p, were measured. Matias-Garcia P R, et al., “Impact of long-term storage and freeze-thawing on eight circulating microRNAs in plasma samples,” PLOS ONE, 15(1):e0227648 (2020).

qPCR of miRNA Using SYBR Green

qPCR for all QC, target and reference miRNA was conducted using Qiagen miRCURY LNA miRNA SYBR PCR Assays (cat. no. 339306) and the miRCURY LNA™ SYBR Green PCR Kit (Qiagen, cat. no. 339347) according to the manufacturer's instructions, and as described previously (Dunlop (2021)). cDNA 1/30 was diluted into nuclease-free water and 3 μL in each 10 μL were used in each qPCR reaction. The pipetting of master mix (7 μL) and samples (3 μL) into 384-well plates was automated using the Opentrons OT-2 liquid-handling robot using a custom protocol written in Python. qPCR was conducted on the Bio-Rad CFX Opus 384 in 384-well plates (Bio-Rad Hard Shell PCR Plates, thin-wall, cat. no. HSP3805). Data in Bio-Rad CFX Maestro version 2.3 were acquired after 40 cycles. Each plate contained triplicate inter-plate calibrators (TATAA IPC, TATAA Biocenter, cat. no. IPC250S) pipetted into the same wells on every plate, to enable comparison of samples run in January 2023 with samples run in August 2023. An IPC correction factor was applied to all samples to enable cross-plate comparison. A melt curve and triplicate no-template controls in each assay were included to check for primer specificity and any nonspecific amplification.

### Hemolysis Calculation

Samples that have undergone hemolysis may show artificially increased miRNA signals, which could distort subsequent results. The gold-standard method for assessing hemolysis is considered a comparison of two miRNA, where miR-23a-3p is stable and not affected by hemolysis while the expression of miR-451a is erythrocyte specific with increased concentrations when hemolysis is present, as described in, for example, Smith M D, et al., “Haemolysis detection in microRNA-seq from clinical plasma samples,” Genes, 13(7):1288 (2022). A delta Cq (ΔCq) for miR-23a-3p minus miR-451a was therefore calculated, where larger values indicate potential hemolysis, as described in, for example, Blondal T, et al, “Assessing sample and miRNA profile quality in serum and plasma or other biofluids,” Methods, 59 (1): S1-6 (2013). Those samples having ΔCq≥7 were selected for further in-depth evaluation according to standard protocols (see Smith (2022) above). The highest result was 8.66 (S141 from January 2023) with a handful of samples returning values ≥7≤8.30. The miRNA Cq values for these samples were otherwise within acceptable ranges as determined using the interquartile range (Q1=30.6, Q3=34.9, IQR=4.3, interquartile multiplier value k=1.5), so they were not excluded from further analysis.

Relative quantitation using 2^{-(ΔΔCq)}

A NormFinder analysis confirmed the stability of miRNAs used for normalization and the same miRNA as those used in prior experiments were selected for consistency and reproducibility (miR-146a-5p, miR-29b-3p, and miR-126-5p). The geometric mean was used for relative quantitation/normalization and a suitability check following the protocol of Vandesompele was performed (Vandesompele J, et al., “Accurate normalization of real-time quantitative RT-PCR data by geometric averaging of multiple internal control genes,” Genome Biol., 3(7):1-12 (2002)). Since the standard deviation of the ratio V 3/4 was less than 0.15, no additional reference miRNAs were needed. Gene fold expression changes were calculated using 2^{-(ΔΔCq)}, with ΔΔCq calculated as the normalized sample Cq value minus the mean of the normalized control sample Cqs. Fold regulation was calculated as relative mean gene fold expression changes in ALS/controls, Parkinson's disease/ALS, and primary lateral sclerosis/ALS, respectively, and fold regulation was defined as equal to gene fold change when greater than one and as negative one divided by gene fold change when fold change was less than one.

### Statistical Analyses

The skewed distribution of the data which varied between miRNA suggested that a non-parametric statistical analyses would be preferable. A Kruskal-Wallis test was performed to assess two alternative hypotheses at the level of p<0.05:
H₀: The gene fold changes, 2^{-(ΔΔCq)}, of miRNA from all disease groups were the same.
H₁: The gene fold changes, 2^{-(ΔΔCq)}, of miRNA from all disease groups were not the same.
   If the null hypothesis H₀ were to be rejected, we would perform post-hoc analyses on gene fold change values (2^{-(ΔΔCq)}) between the following pairs of disease groups, ALS versus Controls, ALS versus primary lateral sclerosis, ALS versus Parkinson's disease, using Mann Whitney tests, at the level of p<0.05 to test the following alternative hypotheses:
H₀: There is no difference in the median gene fold change, 2^{-(ΔΔCq)}, for miRNA between the two disease groups.
H₁: There is a difference in the median gene fold change, 2^{-(ΔΔCq)}, for miRNA between the two disease groups.

Individual miRNA (2^{-(ΔΔCq)}) representing values greater than four standard deviations from the mean were identified as extreme outliers and removed before gene fold expression calculation (one from Parkinson's disease and five from control samples). Three samples were considered invalid and removed entirely from gene fold expression calculations (1 ALS, 1 Control, 1 primary lateral sclerosis) because ≥50% of the miRNA values exceeded four standard deviations from the mean suggesting that the plasma samples from these individuals was compromised. Although nothing was flagged for these samples in the quality control measures built into the methods described herein, plasma samples were collected, processed, and stored initially by numerous individuals across the USA, and consequently, finding three samples of concern out of 331 total represents less than one percent of the total samples analyzed.

Sensitivity and specificity were evaluated using normalized ΔCq values. Sensitivity is a measure of the ability of a screening test to positively classify someone who has the disease. It is calculated as a percent identification of true positive cases in the dataset divided by the total number of individuals with the disease, as defined by a gold standard method of disease identification, described in, for example, Trevethan R, “Sensitivity, Specificity, and Predictive Values: Foundations, Pliabilities, and Pitfalls in Research and Practice,” Front Public Health, 5:30 (2017). The current gold standard for an ALS diagnosis is a clinical evaluation by a neurologist who has evidence of progressive upper and lower motor neuron degeneration as seen in EMG, the ALSFRS-R score, and forced vital capacity (FVC) based upon ratified consensus criteria (Verma A, “Clinical Manifestation and Management of Amyotrophic Lateral Sclerosis in Amyotrophic Lateral Sclerosis. In: Toshiyuki A, ed.,” Amyotrophic Lateral Sclerosis, Exon Publication; 1-14 (2021)). Specificity is a measure of the ability of a screening test to accurately classify someone who does not have the disease, comparing true negative cases to the total number of individuals without the disease in the dataset. Also important for biomarker evaluation is a measure of positive predictive value (PPV) for the test, which compares true positive cases with false positive identification. The negative predictive value (NPV) of a test is the number of true negative cases identified by the test relative to false negative identification (see Trevethan above).

Three orthogonal measures were used to determine ALS classification accuracy against healthy controls in RStudio. First, a logistic regression was calculated to determine a Receiver Operator Characteristics curve (ROC) using only the current data and a random 80% split for training and 20% for testing purposes. Values from all ALS and non-neurological controls (n=269) were included in the model without removing outliers. Seed set was 375 and all miRNA data were included in this model. Second, a random forest machine learning algorithm was used applying all of the current data from this paper (n=269) without removing outliers and a 79% split between training and testing data. The model was tested with a seed set of 375 and iteratively removing miRNA with low model contribution. Third, a second random forest algorithm was used testing the current data set (n=269) against a training set comprised of ΔCq values from an independent cohort previously published (n=100, Banack et al. (2020) Table 3, below). The model was tested with a seed set of 101 and by iteratively removing miRNA with low contributions to the classification. Parkinson's disease and primary lateral sclerosis samples were not included in these models due to sample sizes that were too low to give meaningful results. Hanczar, B, Hua, et al., “Small-sample precision of ROC-related estimates,” Bioinformatics, 26(6):822-830 (2010).

TABLE 3

| | | | | | Data from Banack et | |
|---|---|---|---|---|---|---|
| | | | | | al. 2022 used for | |
| | | | | | Machine Learning | |
| | Amyotrophic | | | [symbol] Primary | Training Data Set | |
| | Lateral | Healthy | Parkinson's | Lateral | | Healthy |
| | Sclerosis | Control | Disease | Sclerosis | ALS | Control |
| Total Samples | 119 | 150 | 20 | 42 | 50 | 50 |
| Male/Female | 72/28 | 110/40 | 14/6 | 24/17 | 35/15 | 30/20 |
| Age Group | | | | | | |
| <30 | 0 | 5 | 0 | 0 | 2 | 14 |
| 30-39 | 3 | 17 | 0 | 1 | | 13 |
| 40-49 | 14 | 15 | 0 | 5 | 7 | [symbol] |
| 50-59 | 21 | [symbol] | 5 | 12 | 19 | 10 |
| 60-69 | 32 | 26 | 3 | 16 | 15 | 4 |
| 70-79 | 28 | 1 | 9 | 4 | 6 | 0 |
| 80-89 | 2 | 0 | 3 | 3 | 1 | 0 |
| ALSFRS-R | | | | | | |
| Mean, Median (Min-Max) | [symbol] | | | | | |
| [symbol] | 50 | | | | | |
| [symbol] | 50 | | | | | |
| Progression | | | | | | |
| [symbol] | [symbol] | | | | [symbol] | |
| [symbol] | 9 | | | | 10 | |
| [symbol] | 0 | | | | 6 | |
| Onset | | | | | | |
| Speech and/or swallowing | [symbol] | | | | 5 | |
| muscles | | | | | | |
| Arm or Hand | 25 | | | | [symbol] | |
| Neck, Back, or Abdominal area | 7 | | | | 0 | |
| Leg or Foot | 35 | | | | 8 | |
| Breathing Muscles | 2 | | | | | |
| All over body | 2 | | | | | |
| ALS recognized in other family | 3 | | | | 0 | |
| members | | | | | | |
| Months since diagnosis | | | | | | |
| Mean, Median (Min-Max) | [symbol] | | [symbol] | [symbol] | | |
| | [symbol] | | [symbol] | [symbol] | | |
| Other | | | | | | |
| [symbol] | | | 13 | | | |
| [symbol] | | | 7 | | | |

[symbol]

indicates data missing or illegible when filed

Table 3 represents Characteristics of study cohort. * Primary Lateral Sclerosis samples from the natural history study (Mitsumoto et al. 2023). +Disease duration at baseline. Amyotrophic Lateral Sclerosis (ALS) progression data were calculated on all samples wherein at least two slopes were provided in the accompanying database. Complete individual characteristics for all patients were not available in the respective databases.

### Results

A comparison of the eight-miRNA ALS fingerprint between 119 ALS and 150 healthy controls indicated identical fold regulation direction with similar magnitude to those found in prior published studies (Table 3 above). A comparison of median gene fold change between ALS, Parkinson's disease, primary lateral sclerosis and healthy controls using a Kruskal-Wallis analysis was highly significant for all eight-miRNA with H values ranging from 38 to 136 (critical value=16, p<0.001), suggesting that at least one of the disease groups was not from the same population. Table 4 below shows the fold regulation of an 8 miRNA ALS fingerprint across 449 independent plasma samples over 4 unique cohort experiments, and reveals remarkable consistency in both direction and values. Negative numbers (in parenthesis) indicated down-regulation of miRNA in ALS relative to controls and positive numbers (in bold) indicated up-regulation. Prior published fold regulation data are from Banack (2020) and Banack (2022).

TABLE 4
Table 4. Fold regulation of an eight miRNA ALS fingerprint
across 449 independent plasma samples over four unique
cohort experiments showed remarkable consistency in both
direction and values. Negative numbers (in parenthesis)
indicated down-regulation of miRNA in ALS relative to controls
and positive numbers indicate up-regulation.

| | Fold Regulation | | | |
|---|---|---|---|---|
| | | | Banack | Banack |
| | | Banack | et al.²² | et al.²² |
| | Current | et al.²³ | Experiment 1 | Experiment 2 |
| | (n = 269) | (n = 100) | (n = 240) | (n = 240) |
| miR-10b-5p | (−7.38) | (−3.2) | (−2.1) | (−7.0) |
| miR-4454 | (−2.55) | (−2.7) | (−1.7) | (−1.8) |
| miR-199a-3p | 2.03 | 1.0 | 1.4 | 2.7 |
| miR-151a-3p | 2.09 | 1.1 | 1.5 | 2.2 |
| miR-151a-5p | 1.77 | 1.1 | 1.4 | 3.2 |
| miR-199a-5p | 2.54 | 1.1 | 1.9 | 4.2 |
| miR-146a-5p | 1.57 | 1.1 | 1.2 | 1.4 |
| miR-29b-3p | (−1.35) | (−1.5) | (−1.7) | (−1.7) |

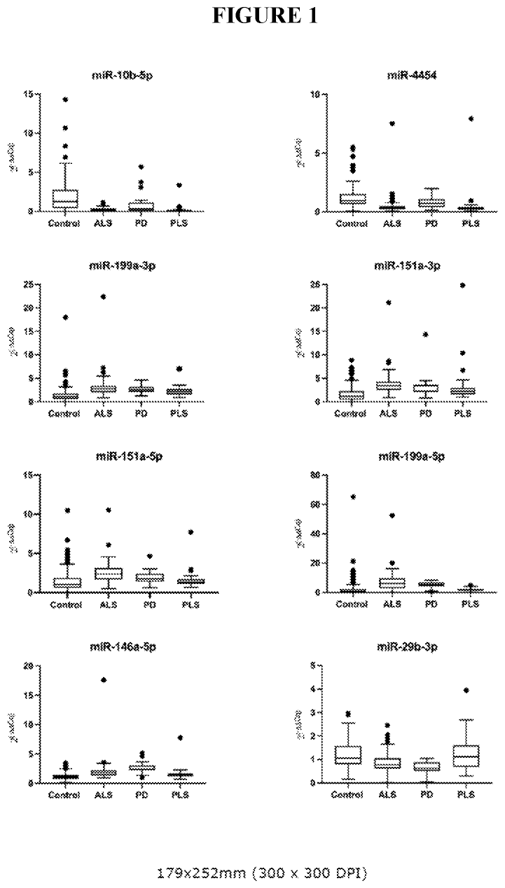

Given the non-normal distribution of miRNA fold change values (FIG. 1), a post-hoc Mann Whitney analyses was used to compare the median gene fold change (2^{-(ΔΔCq)}) for each miRNA for the following disease groups: ALS versus healthy controls, Parkinson's disease versus ALS, and primary lateral sclerosis versus ALS. For all eight-miRNAs, the gene fold change between ALS and control samples had p-values <0.05 (Table 4). The same was true between primary lateral sclerosis and ALS. For the comparison between Parkinson's disease and ALS, five out of eight-miRNAs were different. Interestingly, the three miRNA that were not identified as different between ALS and Parkinson's disease on the Mann Whitney analysis were also not different in another study comparing ALS with controls and may be an artifact of a small sample size. Table 5 provides the fold regulation of ALS miRNA fingerprints, as determined by qPCR from L1CAM enriched EV extractions of blood plasma. Z-statistic was from a two-tailed Mann Whitney post-hoc analysis. Median values are reported as gene fold expression (2^{-(ΔΔCq)}. Z-statistic are reported as absolute values.

TABLE 5
Fold regulation of ALS miRNA fingerprint, as determined by qPCR from L1CAM
enriched EV extractions of blood plasma. Z-statistic was from a two-tailed
Mann Whitney post-hoc analysis. Median values are reported as gene fold expression
(2^{(-ΔΔCq)}). Z-statistic are reported as absolute values.

| | Amyotrophic Lateral Sclerosis (ALS) versus Controls | | | | | |
|---|---|---|---|---|---|---|
| | | | Median | Median | | |
| | | | ALS | Control | Fold | |
| | p-value | Z-statistic | (n = 119) | (n = 150) | Regulation | Regulation |
| miR-10b-5p | p < 0.00001 | 10.39 | 0.15 | 1.27 | (−7.38) | down- |
| | | | | | | regulated |
| miR-4454 | p < 0.00001 | 9.97 | 0.38 | 0.95 | (−2.55) | down- |
| | | | | | | regulated |
| miR-199a-3p | p < 0.00001 | 9.76 | 2.74 | 1.07 | 2.03 | up-regulated |
| miR-151a-3p | p < 0.00001 | 9.49 | 3.39 | 1.18 | 2.09 | up-regulated |
| miR-151a-5p | p < 0.00001 | 8.46 | 2.37 | 1.02 | 1.77 | up-regulated |
| miR-199a-5p | p < 0.00001 | 9.02 | 6.00 | 0.93 | 2.54 | up-regulated |
| miR-146a-5p | p < 0.00001 | 8.66 | 1.81 | 1.04 | 1.57 | up-regulated |
| miR-29b-3p | p < 0.00001 | 4.87 | 0.78 | 1.05 | (−1.35) | down- |
| | | | | | | regulated |

| | Parkinson's Disease (PD) versus ALS | | | | | |
|---|---|---|---|---|---|---|
| | | | Median PD | Median ALS | Fold | |
| | p-value | Z-statistic | (n = 20) | (n = 119) | Regulation | Regulation |
| miR-10b-5p | p < 0.05 | 2.35 | 0.36 | 0.15 | 4.21 | up-regulated |
| miR-4454 | p < 0.001 | 3.58 | 0.72 | 0.38 | 1.64 | up-regulated |
| miR-199a-3p | NS | 0.71 | 2.54 | 2.74 | (−1.06) | down- |
| | | | | | | regulated |
| miR-151a-3p | NS | 0.95 | 3.34 | 3.39 | (−1.20) | down- |
| | | | | | | regulated |
| miR-151a-5p | p < 0.05 | 2.55 | 1.74 | 2.37 | (−1.27) | down- |
| | | | | | | regulated |
| miR-199a-5p | NS | 1.08 | 5.31 | 6.00 | (−1.23) | down- |
| | | | | | | regulated |
| miR-146a-5p | p < 0.00001 | 4.11 | 2.37 | 1.81 | 1.46 | up-regulated |
| miR-29b-3p | p < 0.05 | 2.46 | 0.62 | 0.78 | (−1.37) | down- |
| | | | | | | regulated |
| | Primary Lateral Sclerosis (PLS) versus ALS | | | | | |
| | | | Median PLS | Median ALS | Fold | |
| | p-value | Z-statistic | (n = 42) | (n = 119) | Regulation | Regulation |
| miR-10b-5p | p < 0.05 | 2.41 | 0.08 | 0.15 | (−1.80) | down- |
| | | | | | | regulated |
| miR-4454 | p < 0.05 | 2.32 | 0.27 | 0.38 | (−1.54) | down- |
| | | | | | | regulated |
| miR-199a-3p | p < 0.001 | 3.32 | 2.13 | 2.74 | (−1.32) | down- |
| | | | | | | regulated |
| miR-151a-3p | p < 0.00001 | 4.30 | 2.22 | 3.39 | (−1.37) | down- |
| | | | | | | regulated |
| miR-151a-5p | p < 0.05 | 2.32 | 0.27 | 0.38 | (−1.54) | down- |
| | | | | | | regulated |
| miR-199a-5p | p < 0.00001 | 6.67 | 1.79 | 6.00 | (−3.24) | down- |
| | | | | | | regulated |
| miR-146a-5p | p < 0.001 | 3.63 | 1.50 | 1.81 | (−1.29) | down- |
| | | | | | | regulated |
| miR-29b-3p | p < 0.01 | 2.98 | 1.12 | 0.78 | 1.36 | up-regulated |

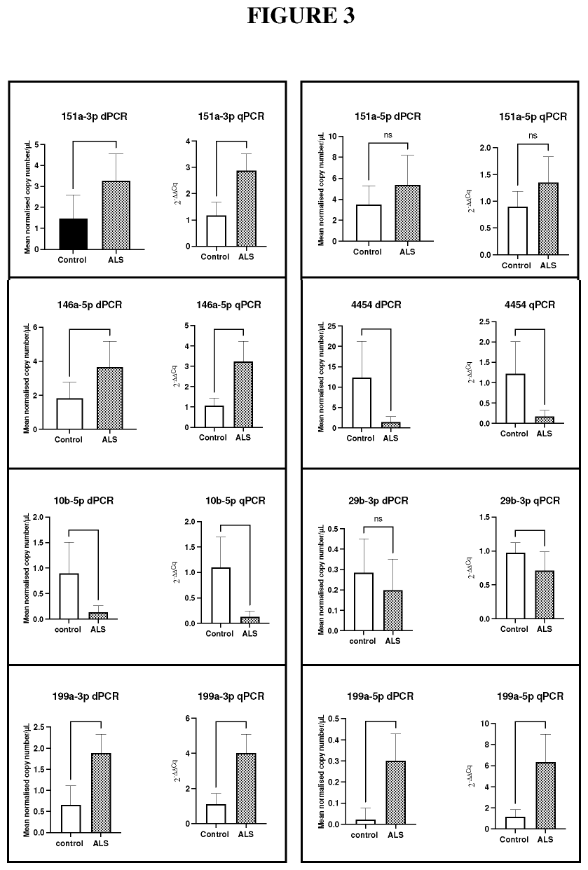

Classification accuracy was tested using three orthogonal measures (Tables 6, 7, 8). Using only the current data of ALS and healthy controls (n=269) and a logistic regression model (Table 7) with a random data split, sensitivity and negative predictive value were both at 100% while specificity and positive predictive value were at 97% and 96% respectively (Table 8). A receiver operator characteristics curve demonstrated an area under the curve of 98% (FIG. 3). Deviance residuals for the model were as follows: Min=−2.10, 1^{rst} Quartile=−0.28, Median=0.03, 3^{rd} Quartile=0.24, Max=3.79, null deviance=296.8 (215 degrees of freedom), residual deviance=96.3 (207 degrees of freedom). Akaike information criterion was 114.3, f1 score was 98%, and the number of Fisher Scoring iterations was 7. A random forest analysis with a random split, also using only the current data, had good sensitivity, specificity, positive predictive value, and negative predictive value all between 96-97% (Table 6). Good results in this analysis used only six of the eight-miRNA (mi-29b-3p and miR-146a-5p were removed) and the contribution of each remaining miRNA was determined (Table 6). When the same data were tested against a training data set pulled from an independent cohort of samples (Banack (2022)), sensitivity dropped to 82% but specificity remained at 97% (Table 8).

Table 6 provides a random forest machine learning analysis, where Mean Decrease in Gini is a measure of the contribution of each miRNA to the homogeneity of the random forest trees where an increased value represents a larger contribution to classification criteria. Random Split data were exclusively from the current data set with a 79% random split. Separate Cohorts used the current data set to test against the training data published in Banack (2022) with used plasma from an independent sample cohort.

TABLE 6
Table 6: Random forest machine learning analysis. Mean Decrease
in Gini is a measure of the contribution of each miRNA to the
homogeneity of the random forest trees where an increased value
represents a larger contribution to classification criteria. Random
Split data are exclusively from the current data set with a 79% random
split. Separate Cohorts used the current data set to test against
the training data, previously published²³, which used plasma
from an independent sample cohort

Mean Decrease Gini

| | | Separate Cohorts: |
|---|---|---|
| | Random Split | Train²³ n = 100; |
| | (n = 269) | Test n = 269 |
| miR-10b-5p | 33.6 | 9.0 |
| miR-4454 | 21.4 | 9.6 |
| miR-199a-3p | 17.4 | 12.2 |
| miR-151a-3p | 15.8 | 4.9 |
| miR-151a-5p | 9.1 | 7.7 |
| miR-199a-5p | 7.8 | 6.0 |

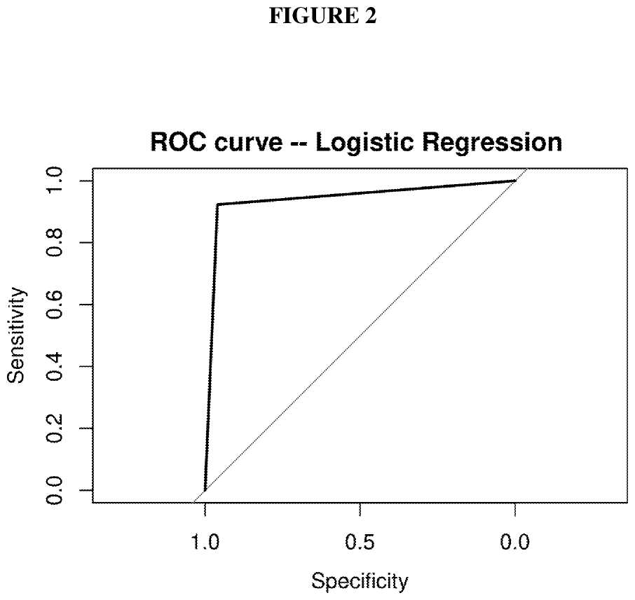

Table 7 below provides the logistic regression model used to calculate the receiver operator characteristics (ROC) curve shown in FIG. 2.

TABLE 7

| | Estimate Std | Std. Error | z value | Pr(>\|z\|) |
|---|---|---|---|---|
| Intercept | −4.58 | 1.76 | −2.61 | 0.009** |
| miR-10b-5p | −0.21 | 0.17 | −1.26 | 0.21 |
| miR-4454 | −1.15 | 0.29 | −3.98 | 6.84e−05*** |
| miR-199a-3p | 1.22 | 0.50 | 2.43 | 0.01* |
| miR-151a-3p | 0.48 | 0.30 | 1.60 | 0.11 |
| miR-151a-5p | 1.17 | 0.37 | 3.19 | 0.001** |
| miR-199a-5p | −0.11 | 0.23 | −0.47 | 0.64 |
| miR-146a-5p | 1.59 | 0.62 | 2.55 | 0.011* |
| miR-29b-3p | 1.72 | 0.46 | 3.70 | 0.0002*** |

***p < 0.001,
**= p < 0.01,
*= p < 0.05

Table 8 below provides results from the three orthogonal models that were used to interrogate ALS classification between ALS and healthy controls.

TABLE 8

| | | | Positive | Negative | Overall | | | Data |
|---|---|---|---|---|---|---|---|---|
| | Sensitivity | Specificity | Predictive | Predictive | Accuracy | # miRNA | Sample | Source |
| | (%) | (%) | Value (%) | Value (%) | (%) | included | Size | (details) |
| 1. Random | 96 | 97 | 96 | 97 | 96 | 6 | n = 269 | Current |
| forest split | | | | | | | | data (79% |
| | | | | | | | | split) |
| 2. Random | 82 | 97 | 95 | 87 | 90 | 6 | n = 369 | Current |
| forest separate | | | | | | | | data (Test, |
| cohorts | | | | | | | | n = 269) + |
| | | | | | | | | Train |
| | | | | | | | | data²³ |
| | | | | | | | | (n = 100) |
| 3. Logistic | 100 | 97 | 96 | 100 | 98 | 8 | n = 269 | Current |
| Regression | | | | | | | | data (80% |
| | | | | | | | | split) |

### Example 2—Using the miRNA Fingerprint of miRNA for Amyotrophic Lateral Sclerosis/Motor Neuron Disease without Isolating Neural-Derived Exosomes (NEEs)

Using retrospective blood samples from previously identified ALS and healthy controls, the eight-miRNA fingerprint of Example 1 was evaluated in circulating blood, without exosome extraction, as an aid to diagnosing ALS.

The majority of ALS samples were obtained from the USA National ALS Biorepository maintained by the Centers for Disease Control and Prevention, and the Agency for Toxic Substances and Disease Registry (CDC, Advarra IRB Pro00053269) with additional ALS blood samples obtained through a Phase IIa clinical trial (NCT03580616, Dartmouth Institutional Review Board D18095, collected between 2019-2022). Control samples were obtained from Innovative Research Inc (Novi, M I, FDA Approval, #3003372368, collected across the USA prior to 2022) and Precision for Medicine (Norton, M A, CR00425931, collected across the USA from 2020-2022) and were all reported to be healthy controls. All blood samples were collected in K2 EDTA tubes and samples blinded before being processed and analyzed. Every patient sample was from a unique individual with no overlap between cohorts.

TABLE 9
Fold regulation values for eight ALS-related miRNAs comparing circulating blood plasma
and neural-enriched extracellular vesicles. Patients were different between cohorts and
therefore there was no overlap between cohorts. The direction of fold-regulation remained
the same between group comparisons (control relative to ALS), but the magnitude of the
fold changes was increased in miR-4454 and miR-10b-5p in the newly developed methods.

| | (NEE miRNA) | | | | | | | | |
|---|---|---|---|---|---|---|---|---|---|
| | Banack | Banack | Banack | Banack | | | | | |
| | et al. | et al. | et al. | et al. | | | | | |
| | 2020 | 2020 | 2022 | 2024 | (Circulating miRNA) | | | | |
| | Cohort | Cohort | Cohort | Cohort | Cohort | Cohort | Cohort | Cohort | Cohort |
| | 1 | 2 | 3 | 4 | 1 | 2 | 3 | 4 | 5 |
| 151a-3p | 1.5 | 2.2 | 1.1 | 2.1 | 3.5 | 1.8 | 3.0 | 3.0 | 3.1 |
| 151a-5p | 1.4 | 3.2 | 1.1 | 1.8 | 2.3 | 2.2 | 2.4 | 3.4 | 4.5 |
| 146a-5p | 1.2 | 1.4 | 1.1 | 1.6 | 2.3 | 1.5 | 2.2 | 1.5 | 1.5 |
| 4454 | (−1.7) | (−1.8) | (−2.7) | (−2.6) | (−10.3) | (−12.3) | (−9.0) | (−4.6) | (−10.0) |
| 10b-5p | (−2.1) | (−7.0) | (−3.2) | (−7.4) | (−23.0) | (−14.0) | (−14.8) | (−14.5) | (−24.4) |
| 199a-3p | 1.4 | 2.7 | 1 | 2.0 | 2.7 | 1.9 | 3.4 | 2.8 | 2.6 |
| 199a-5p | 1.9 | 4.2 | 1.1 | 2.5 | 3.8 | 2.8 | 6.7 | 5.6 | 4.6 |
| 29b-3p | (−1.7) | (−1.7) | (−1.5) | (−1.4) | (−1.3) | (−1.2) | (−1.5) | (−1.2) | (−1.4) |

| | | | | | Totals | | | | | | Totals |
|---|---|---|---|---|---|---|---|---|---|---|---|
| n = ALS | 10 | 10 | 50 | 119 | 189 | 20 | 132 | 59 | 82 | 100 | 393 |
| n = Controls | 10 | 10 | 50 | 150 | 220 | 20 | 133 | 60 | 82 | 100 | 395 |

The data from “Cohort 1” in the circulating miRNA method were analyzed from blood plasma miRNA representing 20 ALS patients and 20 controls. The data from “Cohort 2” included 132 ALS patient plasma and 133 controls. “Cohort 3” came from 59 ALS patients and 60 controls. The data from “Cohort 4” came from 82 ALS patients and 82 controls and “Cohort 5” came from 100 ALS and 100 controls.

### RNA Extraction

In order to compare the circulating miRNA results with previous data obtained from isolated exosomes (“NEE”), the same plasma samples of Example 2 extracted total RNA without first isolating NEE. A slightly different protocol for total RNA extraction from plasma which varies by the volume of lysis reagent and number and volume of ethanol washes was used.

Control and ALS plasma was removed from an −80 °C freezer, thawed at room temperature, then 200 μL aliquoted into a 1.5 mL LoBind® Tube (Eppendorf). Total RNA was extracted using the Qiagen miRNeasy Serum/Plasma Kit (50) Cat. No./ID: 217184 according to the manufacturer's instructions. Briefly, 1 mL Qiazol Lysis buffer, containing 1 μL UniSp2, 4 and 5 spike-in, was added to each sample and mixed by pipetting up and down several times. Samples were incubated at RT for 5 mins. An equal volume of chloroform (200 μL) was added to each sample, then the tubes were vigorously shaken for 15 seconds. The samples were incubated at RT for 3 mins, then centrifuged at 12,000×g for 15 min at 4 °C, to generate three distinct layers. The top aqueous layer containing RNA was carefully removed without touching the middle layer containing DNA and placed in a new clean 2 mL LoBind® tube. To this were added 1.5 volumes of 100% ethanol (to 700 μL RNA was added 1050 μL 100% ethanol). The samples were mixed thoroughly by pipetting up and down. To a spin column, 700 μL sample was added, then centrifuged at 10,000×g for 15 secs at RT, then the eluent was discarded. This step was repeated until all the sample had been added to the spin column (three spins).

About 700 μL Buffer RWT then was pipetted into each column, then spun at 10,000×g for 15 secs at RT. The eluent was discarded. Next, about 500 μL Buffer RPE was added to the spin-column then centrifuged at 10,000×g for 15 secs at RT, and the eluent was discarded. About 500 μL 80% ethanol then was added to the spin column then spun for 2 min at 10,000×g at RT, and the eluent discarded. The spin-column was placed in a new 2 mL collection tube (with no lid), and centrifuged at 17,000×g for 5 min, RT to dry the spin column. Finally, the spin column was placed in a clean 1.5 mL LoBind® tube, and 15 μL nuclease-free water added directly to the membrane. The spin-columns were spun for 1 min at 17,000×g to elute the RNA. The RNA was frozen at −80 °C until required for cDNA synthesis.

### CDNA Synthesis

cDNA was synthesised as described previously (Dunlop et al., 2021), and according to the manufacturer's instructions using the Qiagen miRCURY LNA RT Kit (#339340). cDNA was synthesised in duplicate, and to each 10 μL reaction were added 0.5 μL of UniSp6 and cel-miR-39-3p spike-ins (UniSp6 is a part of the miRCURY LNA RT Kit #339340 and cel-miR-39-3p is part of the miRCURY LNA RNA Spike-in Kit #339390. For instructions on how to reconstitute these, see RNA Spike-in Kit, for RT, Handbook #HB-2433-002). About 4 μL total RNA was used in the cDNA synthesis reaction, since it was previously demonstrated that 4 μL returns a more robust Cq in genes of low copy number (Dunlop et al., 2021). The conditions for the RT reaction were as follows: 95 °C. 5 min, 42 °C. 60 mins, 4 °C. HOLD. Duplicate cDNA reactions were pooled into a final volume of 20 μl, then aliquoted into 3 μL aliquots before being stored at −20 °C.

For each sample, quality control was conducted prior to downstream qPCR analysis. Each sample was assayed for the following spike-ins; UniSp2, 4 and 5 to measure the effectiveness of the RNA extraction step, and UniSp6 and cel-miR-39-3p to measure the success of the cDNA/RT reaction step. All samples were deemed to have passed QC if the Cqs for spike-ins varied by less than two or three standard deviations from the mean raw Cq for each target.

### Sample Signal

As a method for determining whether there was sufficient cDNA in each sample to proceed with downstream reactions, five miRNAs were measured that were collectively referred to as “sample signal.” Five miRNA that were predicted to be either in low or high copy-number in human plasma were measured; miR-142-3p, miR-451a, miR-23a-3p, miR-30c-5p, and miR-103a-3p. The presence of these targets with raw Cqs≤34 constituted a “pass” for use in downstream experiments.

### Hemolysis Calculation

Certain miRNAs are enriched in red blood cells, and if haemolysis occurs during plasma processing, the release of these miRNAs into the plasma can artefactually affect results. Thus, miR-451a, miR-23a-3p were measured, and ΔCq for (miR-23a-3p-miR-451a) was calculated. Any ΔCq that was ≥7 indicated haemolysis may have occurred and these samples should be assessed.

### Efficiency Calculations

The efficiency of primer pairs to amplify the target was calculated in two ways: Using the standard curve method, and the LinRegPCR method. For the standard curve method, cDNA was randomly pooled from the total sample set. A cDNA dilution series of six samples diluted 1/2 was prepared from an initial dilution of ⅕ into nuclease-free water. Standard curves were run in triplicate, and data acquired in Maestro version 2,3 on the BioRad Opus CFX 384. Reaction conditions were as follows: initial activation, 2 min, 95 °C; 2-step cycling: denaturation, 10 secs, 95 °C; combined annealing/extension; 60 secs, 56 °C; for a total of 40 cycles. Efficiency was calculated by Maestro 2.3 from the slope of each standard curve. Primer efficiencies between 94% and 102.7% were achieved. Individual reaction efficiencies for every sample were also calculated in LinRegPCR (Untergasser A. et al., Web-based LinRegPCR: application for the visualization and analysis of (RT)-qPCR amplification and melting data. BMC Bioinformatics; 22:398. 2021) using the slope determined from the window-of-linearity for each individual reaction. An average PCR efficiency was calculated for all samples in an amplicon group, and the average efficiencies for all primer pairs should be between 1.80 and 2.00.

### RNA Extraction Efficiency

Prior to lysing plasma samples, 1 μL of combined UniSp2, Unip4 and UniSp5 synthetic spike-in was added to each 1 mL of Qiagen QIAzol® Lysis Reagent (#79306, Lot No. 57204842). Spike-ins were prepared according to the manufacturer's instructions (Qiagen miRCURY LNA RNA Spike-in Kit #339390, Lot No. PA03). RNA extraction efficiency was determined by measuring UniSp2, 4 and 5 in all samples by qPCR using miRCURY LNA miRNA PCR assays #339306.

Real-Time PCR (qPCR) and Analysis

cDNA was removed from the freezer (−20 °C) and diluted 1/30 into nuclease-free water. qPCR was conducted using Qiagen miRCURY LNA PCR Assays #339306 according to the manufacturer's instructions. Preparation of 384-well PCR plates was conducted on the Opentrons OT-2 liquid-handling robot using a custom protocol designed in Python 3.0. To 7 μL master mix, 3 μL sample was added to each well and mixed five times using the robot. Each qPCR plate contained three no-template controls per gene and triplicate inter-plate calibrators to allow for cross-plate comparison (TATAA, Sweden). qPCR was run on the BioRad CFX Opus 384 using the following conditions: initial activation, 2 min, 95 °C; 2-step cycling: denaturation, 10 secs, 95 °C; combined annealing/extension; 60 secs, 56 °C; for a total of 40 cycles. A melt-curve (60-95 °C) for each primer was used to check for non-specific amplification using the sample maximization method. Cq accounting for efficiency was determined from the double derivative method using LinRegPCR (Untergasser A. et al., Web-based LinRegPCR: application for the visualization and analysis of (RT)-qPCR amplification and melting data. BMC Bioinformatics; 22:398. 2021).

Cq values were normalized using the geometric mean of three stable reference genes (miR-146a-5p, miR-29b-3p, and miR-126-5p) and a suitability test was calculated to assess suitability (Banack et al. 2024, Vandesompele et al. 2002). Gene-fold expression, 2^{-(ΔΔCq)}, was calculated from delta-delta Cq (normalized sample Cq minus the mean of normalized control sample Cqs). Fold regulation was calculated using the mean gene fold expression for ALS divided by the mean gene fold expression for control samples. For this analyses values greater than four standard deviations were considered extreme outliers and were eliminated from the analysis for that specific miRNA alone (n=0 for circulating cohort 1; n=6 for circulating cohort 2; and n=8 for circulating cohort 3). Fold regulation was defined as equal to gene fold change when it was greater than one and was calculated as negative one divided by gene fold change when the fold change was less than one.

Digital PCR (dPCR)

Total RNA was extracted from 200 μL plasma samples and cDNA synthesized as described above. Twenty-four of these RNA samples were simultaneously synthesized in triplicate into cDNA, and from the extra reaction, 2 μL was aliquoted and diluted with 58 μL water to make a dilution of 1/30. Diluted samples were frozen at −20 °C until needed. For the dPCR reaction, an 8500 partition 96-well plate was used on the Qiagen QIAcuity instrument. In each 15 μL dPCR reaction, 3 μL of diluted cDNA and 1.5 μL primer was used (1/10 dilution, as per the qPCR reaction). To allow for direct comparison, the exact same primers were used for this experiment at the same dilution (1/10) as qPCR comparisons. dPCR and qPCR data were normalized, to account for variations across samples in RNA concentrations, using primers 146a-5p, 29b-3p, and 126-5p. The normalization was performed using equations 1 through 4. dPCR data was not obtained for all samples with primer 29b-3p; those samples without a measured copies per microliter were assigned a copies per microliter value of 0.25 thus facilitating the calculation of a geometric mean.

$$( dPCR data normalization ) Δ {C}_{qprimer i , sample j} = {C}_{qprimer i , sample j} - {C}_{qgeometricmean , sample j} \qquad Eq . 1$$

$$( dPCR data normalization ) {β}_{primer i , sample j} = \frac{{α}_{primer i , sample j}}{{α}_{geometricmean , sample j}} \qquad Eq . 2$$

$${C}_{qgeometricmean , sample j} = {( {C}_{q 146 a - 5 p , sample j} \times {C}_{q 29 b - 3 p , sample j} \times {C}_{q 126 - 5 p , sample j} )}^{1 / 3} \qquad Eq . 3$$

$${α}_{geometricmean , sample j} = {( {α}_{146 a - 5 p , sample j} \times {α}_{29 b - 3 p , sample j} \times {α}_{126 - 5 p , sample j} )}^{1 / 3} \qquad Eq . 4$$

where: ΔCq is the concentration normalized Cq
α is the measured copies per microliter
β is the concentration normalized copies per microliter

Fold regulation was calculated for qPCR and dPCR using Equations 5 through 9.

ΔΔC_{qprimeri,samplej}=ΔC_{qprimeri,samplej}−medianΔC_{qprimeri,forallcontrolsamples} Eq. 5

Foldregulation(qPCR)=medianALS2^{-ΔΔCqprimeri}/mediancontrol2^{-ΔΔCqprimeri} Eq. 6

Δβ_{primeri,samplej}=β_{primeri,samplej}−medianβ_{primeri,forallcontrolsamples} Eq. 7

Foldregulation(dPCR)=medianALS2^{-Δβprimeri}/mediancontrol2^{-ΔΔprimeri} Eq. 8

when Foldregulation(qPCR V dPCR)<1Foldregulation=1/−Foldregulation Eq. 9

A comparison between qPCR and dPCR was made for eight miRNAs, and is shown in FIG. 3. As shown in the Figure, all miRNAs that were previously statistically significant and differentiated ALS from controls were also highly significant when quantitated using dPCR. All samples were normalized to a geometric mean of 146a-5p, 29b-3p, and 126-5p. Unpaired non-parametric T-test, Mann-Whitney post-hoc analysis in GraphPad Prism version 10 was used.

Fold-regulation direction was the same in dPCR, but the change was of smaller magnitude in upregulated genes and larger in down-regulated genes, as shown in Table 10 below. The fold regulation in Table 10 was defined as equal to fold change (median ALS/median Control) when greater than one, and as negative one divided by fold change when fold change was less than one. The direction of fold-regulation was mostly reproduced for dPCR, but the magnitude was smaller for positive changes, and larger for negative changes.

TABLE 10

| | 151a-3p | 151a-5p | 146a-5p | 4454 | 10b-5p | 29b-3p | 199a-3p | 199a-5p | 107 | 150-5p | let-7g-5p | 126-5p |
|---|---|---|---|---|---|---|---|---|---|---|---|---|
| Fold | 1.88 | 1.07 | 1.27 | −8.84 | −5.84 | −1.01 | 2.50 | 1.80 | −1.18 | −13.43 | −3.65 | −2.63 |
| regulation | | | | | | | | | | | | |
| dPCR | | | | | | | | | | | | |
| Fold | 2.92 | 1.14 | 3.21 | −7.8 | −10.55 | −1.55 | 4.04 | 6.04 | −1.43 | −7.46 | −2.82 | −1.9 |
| regulation | | | | | | | | | | | | |
| qPCR | | | | | | | | | | | | |

### Classification Algorithms

The inventors used a cross-validation logistic regression model to evaluate classification accuracy on samples from 393 ALS patients and samples from 395 healthy controls (e.g., without evidence of neurological disease). Since machine learning classification algorithms are sensitive to sample size, it was important to create an algorithm on the complete dataset from five independently analyzed cohorts. Classification analysis was performed using R Statistical Software v4.1.2. A logistic regression was conducted on non-IPC corrected, ΔCq data with a Lasso feature selection using the following model parameters: seed set 42, multinomial family, MSE measure, Z-score standardization, relaxed glm fit without penalty, lambda sequence (−4, −1, length.out=1000, min), 40 nfolds, 1 e⁻⁷ convergence threshold, and 1 e⁵ maximum iterations (eMethods-Supplement 1). All 8-miRNA identified as the ALS fingerprint were considered for model inclusion (miR-151a-3p, miR-151a-5p, miR-4454, miR-146a-5p, miR-10b-5p, miR-199a-3p, miR-199a-5p, miR-29b-3p).

The model used the combined contribution of six miRNAs, 151a-3p, 151a-5p, 4454, 10b-5p, 199a-5p, 29b-3p (Table 11) as sufficient to classify the 788 samples with 0.98 AUC (95% CI 97-99), 95% balanced accuracy (95% CI 93-97), 97% sensitivity (95% CI 96-98), and 93% specificity (95% CI 90-96). The positive and negative predictive values of the model were 0.93 (95% CI 0.91-0.96), and 0.97 (95% CI 0.96-0.98), respectively.

Table 11: The coefficients of the cross-validation logistic regression model used for sample classification. Data were calculated using ΔCq values.

TABLE 11

| | ALS | Control |
|---|---|---|
| (Intercept) | 3.11 | −3.11 |
| miR-151a-3p | −0.69 | 0.69 |
| miR-151a-5p | −0.46 | 0.46 |
| miR-4454 | 0.32 | −0.32 |
| miR-10b-5p | 0.13 | −0.13 |
| miR-199a-5p | −0.36 | 0.36 |
| miR-29b-3p | −0.87 | 0.87 |

In order to predict the probability that the individual who provided a blood sample is at risk of having a neurological disease, a combination of ΔCq values from the individual are created weighting the value of each miRNA contribution relative to its predictive value as determined by a logistic regression model, random forest machine learning classification model, or other classification software, and our proprietary classification algorithm. The classification algorithm is trained on data consisting of ΔCq values obtained for each miRNA from hundreds of individual blood samples with known clinical diagnosis, either diseased or normal healthy control. In the classification algorithm model, the predictive contribution of each miRNA is evaluated, and a classification algorithm assigns a coefficient which designates the predictive value for that specific miRNA. In the model, the dependent variable can be binary indicating two possible outcomes (ALS or not-ALS) and/or the dependent variable can be multi-factorial indicating the assignment to a specific class of disease among several choices (eg. ALS, PD, PLS, Control). The dependent variable represents the probability that the blood sample came from one of the tested classes. The independent variables in the model are the ΔCq values of each miRNA.

The algorithm equation predicts the probability that the blood sample came from someone with ALS or not-ALS or other combinations. The coefficients, which define the model, indicate the change in the likelihood of the outcome per one-unit change in the predictor variable, while holding all other variables constant. The coefficients developed are based on our extensive laboratory research combining the measured miRNA concentrations of hundreds of individual patients and determining which miRNA change as the disease develops in an individual. However, since one miRNA can interact with multiple physiological pathways, the absolute quantification as measured by either Cq or ΔCq is insufficient. Therefore, it is not the quantification of miRNAs alone that determines the disease state, but the relative contribution of related miRNA. Determining the combination of miRNA which are important for each disease is critical as any one miRNA alone can reflect a multitude of physiological changes due to other health indications or medications within any given individual. Importantly, the results of our proprietary algorithm may suggest that the person involved does not have a particular neurological disease.

In order to determine the coefficients for the algorithm, we modeled multiple cohorts of diseased and normal blood samples which enabled us to identify unique combinations of miRNA sequences and the combined relative contribution of each miRNA to specific disease predictions. Once the specific miRNA and their coefficients are identified, any subsequent blood sample can then be compared against the classification algorithm to determine the probability that the sample came from someone with the particular disease of interest.

The coefficient ranges useful to diagnose ALS were calculated based on analysis of hundreds of ALS and normal control blood samples. The range comes from the analyses of different cohorts using the combined importance of ΔCq values in determining group classification (Table 12).

TABLE 12

| miRNA | ALS | Control |
|---|---|---|
| miR-151a-3p | −0.12 to −0.86 | 0.12 to 0.86 |
| miR-151a-5p | −0.29 to −0.79 | 0.29 to 0.79 |
| miR-4454 | 0.20 to 0.48 | −0.20 to −0.48 |
| miR-10b-5p | 0.06 to 0.34 | −0.06 to −0.34 |
| miR-199a-5p | 0.02 to −0.72 | −0.02 to 0.72 |
| miR-29b-3p | −0.87 to −1.3 | 0.87 to 1.3 |
| miR-146a-5p | −0.11 to −0.65 | 0.11 to 0.65 |
| miR-199a-3p | −0.70 to 0.44 | 0.70 to −0.44 |

Data also were analyzed to determine the classification efficiency between: (a) PLS healthy control samples; (b) PLS and ALS; and (c) PLS and Parkinson's Disease. The results are shown in Table 13 below.

TABLE 13

| | | | | | | False | False | | |
|---|---|---|---|---|---|---|---|---|---|
| | | | | | | Negative = | positive = do | | |
| | | | | | | have PLS | not have | | |
| PLS | | | | | | but test says | PLS but test | % overall | |
| Sample Size | Condition | Sensitivity | Specificity | PPV | NPV | not | says you do | accuracy | Kappa |
| 67 | Control, n = 183 | 100 | 100 | 100 | 100 | 0/13 | 0/36 | 100 | 1 |
| 67 | ALS, n = 137 | 92 | 89 | 80 | 96 | 1/13 | 3/27 | 90 | 0.78 |
| 67 | PD, n = 26 | 100 | 100 | 100 | 100 | 0/13 | 0/5 | 100 | 1 |

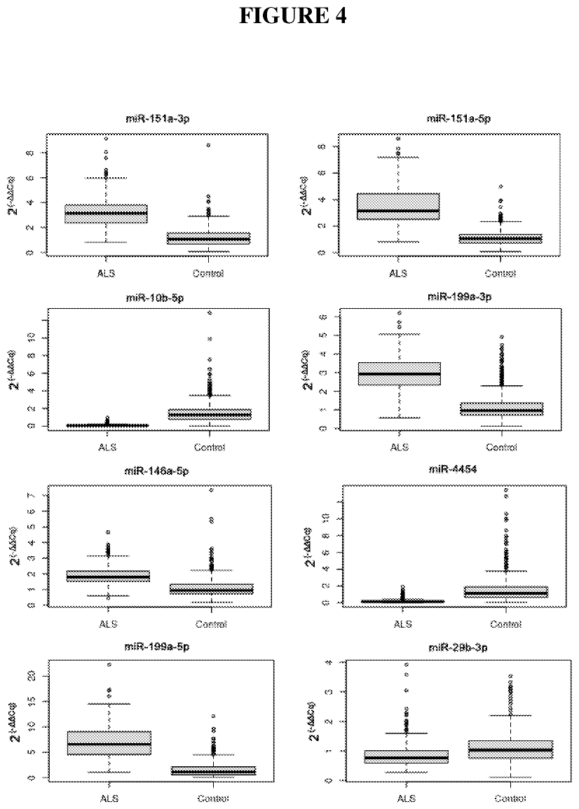

The above example was repeated based on 788 ALS patients and controls, and the box and whisker plots of the 8-miRNA developed using the model of Example 1 were included as variables in a cross-validation logistic regression model for ALS. The box and whisker plots are provided in FIG. 4. A cross-validation logistic regression model was used to evaluate classification accuracy on samples from 393 ALS patients and samples from 395 healthy controls (e.g., without evidence of neurological disease). The model identified six miRNAs of the eight entered (Table 3) as sufficient to classify the 788 samples with 0.98 AUC (95% CI 97-99), 95% balanced accuracy (95% CI 93-97), 97% sensitivity (95% CI 96-98), and 93% specificity (95% CI 90-96). The positive and negative predictive values of the model were 0.93 (95% CI 0.91-0.96), and 0.97 (95% CI 0.96-0.98), respectively

Using the techniques described herein, ALS could be detected and differentiated from other neurological disorders, including primary lateral sclerosis (0.92 AUC), multiple sclerosis (0.99 AUC), Parkinson's disease (0.97 AUC), and Lyme disease (0.94 AUC). All individuals with known ALS genetic mutations were correctly classified as ALS with this diagnostic test (20 C9orf, 3 SOD1, 1 ALS2, 1VAPD, 2 SETX, 2 “Other”).

A method of developing a model for a neurodegenerative disorder may begin with determining a model using neural derived exosome fractions, as described above, and testing that model with results from extracting miRNA from other bodily fluids. For ALS, the method may include extracting extracellular vesicles from venous blood samples, and enriching a neural fraction by contact with L1CAM antibody, and isolating that fraction. The method may include extracting RNA from the enriched fraction, synthesizing the cDNA of the extracted RNA, and amplifying the extracted miRNA using qPCR to a quantifiable fraction and assigning each of the miRNA an Cq value. The Cq values then can be normalized using the geometric mean of suitable endogenous reference genes to obtain ΔCq values for each of the miRNA in the model. The relative contribution of each miRNA then can be determined by weighting the ΔCq values of each miRNA relative to its predictive value as determined by logistic regression analysis against a library of previously determined ΔCq values obtained from each miRNA extracted from samples from individuals with known clinical diagnosis, either ALS or a healthy control.

The method then may assign a coefficient to each of the miRNA based on the relative contribution of each miRNA, the coefficient designating each miRNA's predictive value, and comparing the coefficients of the miRNA to the coefficients in a library of previously determined coefficients from each miRNA extracted from samples from individuals with known clinical diagnosis, either ALS or a healthy control. The subject may be diagnosed with ALS when two or more, or three or more, or four or more, or five or more, or six or more of the miRNA in the model have coefficients that substantially match the coefficients of the same miRNA in the library of previously determined coefficients.

### Example 3—Developing an miRNA Fingerprint of miRNA for Alzheimer's Disease and/or Mild Cognitive Impairment

Plasma samples were collected in K2 EDTA tubes from a total of 126 AD subjects, 66 MCI subjects, and 224 healthy subjects and were separated into three cohort groups. The first Cohort included 39 AD, 39 MCI and 42 control samples; the second Cohort included 62 AD, 21 MCI and 82 control samples; and the third Cohort included 25 AD, 6 MCI, and 100 control samples. The AD samples were obtained from Precision for Medicine (Norton, MA, CR00425931, collected across the USA) and Dartmouth Hitchcock Medical School (NCT03062449), MCI samples were obtained from Houston Methodist's Neurological Institute (HMCI-2), and healthy control samples were obtained from Innovative Research Inc (Novi, MI, FDA Approval, #3003372368, collected from across the USA) and Precision for Medicine (Norton, MA, CR00425931, collected from across the USA). All blood samples were collected in K2 EDTA tubes and samples blinded before being processed and analyzed. Every patient sample was from a unique individual with no overlap between cohorts.

### RNA Extraction

Control and AD and/or MCI plasma was removed from an −80 °C freezer, thawed at room temperature, then 200 μL aliquoted into a 1.5 mL LoBind® Tube (Eppendorf). Total RNA was extracted using the Qiagen miRNeasy Serum/Plasma Kit (50) Cat. No./ID: 217184 according to the manufacturer's instructions. Briefly, 1 mL Qiazol Lysis buffer, containing 1 μL UniSp2, 4 and 5 spike-in, was added to each sample and mixed by pipetting up and down several times. Samples were incubated at room temperature (RT) for 5 mins. About 200 μL of chloroform was added to each sample, then the tubes were vigorously shaken for 15 seconds. The samples were incubated at RT for 3 mins, then centrifuged at 12,000×g for 15 min at 4 °C, to generate three distinct layers. The top aqueous layer containing RNA was carefully removed without touching the middle layer containing DNA and placed in a new clean 2 mL LoBind® tube. To this were added 1.5 volumes of 100% ethanol (to 700 μL RNA was added 1050 μL 100% ethanol). The samples were mixed thoroughly by pipetting up and down. To a spin column, 700 μL sample was added, then centrifuged at 10,000×g for 15 secs at RT, then the eluent was discarded. This step was repeated until all the sample had been added to the spin column (three spins).

About 700 μL Buffer RWT then was pipetted into each column, then spun at 10,000×g for 15 secs at RT. The eluent was discarded. Next, about 500 μL of Buffer RPE was added to the spin-column then centrifuged at 10,000×g for 15 secs at RT, and the eluent was discarded. About 500 μL 80% ethanol then was added to the spin column then spun for 2 min at 10,000×g at RT, and the eluent discarded. The spin-column was placed in a new 2 mL collection tube (with no lid), and centrifuged at 17,000×g for 5 min, RT to dry the spin column. Finally, the spin column was placed in a clean 1.5 mL LoBind® tube, and 15 μL nuclease-free water added directly to the membrane. The spin-columns were spun for 1 min at 17,000×g to elute the RNA. The RNA was frozen at −80 °C until required for cDNA synthesis.

### CDNA Synthesis

cDNA was synthesised as described previously (Dunlop et al., 2021), and according to the manufacturer's instructions using the Qiagen miRCURY LNA RT Kit (#339340). cDNA was synthesised in duplicate, and to each 10 μL reaction were added 0.5 μL of UniSp6 and cel-miR-39-3p spike-ins (UniSp6 is a part of the miRCURY LNA RT Kit #339340 and cel-miR-39-3p is part of the miRCURY LNA RNA Spike-in Kit #339390. For instructions on how to reconstitute these, see RNA Spike-in Kit, for RT, Handbook #HB-2433-002). About 4 μL total RNA was used in the cDNA synthesis reaction, since it was previously demonstrated that 4 μL returns a more robust Cq in genes of low copy number (Dunlop et al., 2021). The conditions for the RT reaction were as follows: 95 °C. 5 min, 42 °C 60 mins, 4 °C. HOLD. Duplicate cDNA reactions were pooled into a final volume of 20 μl, then aliquoted into 3 μL aliquots before being stored at −20 °C.

For each sample, quality control was conducted prior to downstream qPCR analysis. Each sample was assayed for the following spike-ins; UniSp2, 4 and 5 to measure the effectiveness of the RNA extraction step, and UniSp6 and cel-miR-39-3p to measure the success of the cDNA/RT reaction step. All samples were deemed to have passed QC if the Cqs for spike-ins varied by less than two standard deviations from the mean raw Cq for each target.

### Sample Signal

As a method for determining whether there was sufficient cDNA in each sample to proceed with downstream reactions, five miRNAs were measured that were collectively referred to as “sample signal.” Five miRNA that were predicted to be either in low or high copy-number in human plasma were measured; miR-142-3p, miR-451a, miR-23a-3p, miR-30c-5p, and miR-103a-3p. The presence of these targets with raw Cqs≤34 constituted a “pass” for use in downstream experiments.

### Haemolysis Calculation

Certain miRNAs are enriched in red blood cells, and if haemolysis occurs during plasma processing, the release of these miRNAs into the plasma can artefactually affect results. Thus, miR-451a, miR-23a-3p were measured, and ΔCq for miR-23a-3p-miR-451a was calculated. Any ΔCq that was ≥7 indicated haemolysis may have occurred and these samples would be assessed.

Real-Time PCR (qPCR) and Analysis

cDNA was removed from the freezer (−20 °C) and diluted 1/30 into nuclease-free water. qPCR was conducted using Qiagen miRCURY LNA PCR Assays #339306 according to the manufacturer's instructions. Preparation of 384-well PCR plates was conducted on the Opentrons OT-2 liquid-handling robot using a custom protocol designed in Python 3.0. To 7 μL master mix, 3 μL sample was added to each well and mixed five times using the robot. Each plate contained three no-template controls per gene and triplicate inter-plate calibrators to allow for cross-plate comparison (TATAA, Sweden). qPCR was run on the BioRad CFX Opus 384 using the following conditions: initial activation, 2 min, 95 °C; 2-step cycling: denaturation, 10 secs, 95 °C; combined annealing/extension; 60 secs, 56 °C; for a total of 40 cycles. A melt-curve (60-95 °C) for each primer was used to check for non-specific amplification using the sample maximization method. Cq accounting for efficiency was determined from the double derivative method using LinRegPCR (Untergasser A. et al., Web-based LinRegPCR: application for the visualization and analysis of (RT)-qPCR amplification and melting data. BMC Bioinformatics; 22:398. 2021).

Cq values were normalized using the geometric mean of three stable reference genes (miR-146a-5p, miR-29b-3p, and miR-126-5p) and a suitability test was calculated to assess suitability (Banack et al. 2024, Vandesompele et al. 2002). Gene-fold expression, 2^{-(ΔΔCq)}, was calculated from delta-delta Cq (normalized sample Cq minus the mean of normalized control sample Cqs). Fold regulation was calculated using the mean gene fold expression for ALS divided by the mean gene fold expression for control samples. For this analyses values greater than four standard deviations were considered extreme outliers and were eliminated from the analysis for that specific miRNA alone (n=0 for circulating cohort 1; n=6 for circulating cohort 2; and n=8 for circulating cohort 3). Fold regulation was defined as equal to gene fold change when it was greater than one and was calculated as negative one divided by gene fold change when the fold change was less than one.

The data from this analysis was used to compare miRNA from AD and/or MCI subjects to healthy control subjects to assess the fold difference, and those miRNAs that exhibited sufficiently consistent differences are provided in Table 14 below. The data also was used to compare AD and MCI subjects (MCI subjects in this instance could be considered a control) to assess the fold differences in miRNA and thereby provide an miRNA fingerprint that could be used to assess whether a subject was, or was at risk of progressing from MCI to AD. The miRNA that showed sufficiently consistent fold changes (e.g., increases or decreases) along with the respective fold changes are provided in Table 14 below:

TABLE 14

| | AD/Control | | | | | |
|---|---|---|---|---|---|---|
| | | | Cohort 2 + 3 | | MCI/Control | |
| | Cohort 1 | | 87 AD (62 + 25)/ | | Cohort 1 | |
| | 39 AD/42 Control | | 182 Control (82 + 100) | | 39 MCI/42 Control | |
| | up | down | up | down | up | down |
| miRNA | regulated | regulated | regulated | regulated | regulated | regulated |
| 151a-3p | 1.99 | | 2.12 | | 2.01 | |
| 151a-5p | 1.23 | | 1.87 | | 2.22 | |
| 146a-5p | 2.22 | | 1.45 | | 1.61 | |
| 4454 | | −4.25 | | −2.31 | | −8.96 |
| 10b-5p | | −3.70 | | −3.33 | | −5.03 |
| 199a-3p | 1.67 | | 1.75 | | 1.79 | |
| 199a-5p | 1.63 | | 2.92 | | 2.21 | |
| 29b-3p | | −2.03 | 1.00 | | | −1.30 |
| 126-5p | | −1.21 | | −1.33 | | −1.29 |
| 150-5p | | −5.74 | | −3.83 | | −6.15 |
| 107 | | −1.24 | 1.75 | | 1.78 | |
| let-7g-5p | | −1.32 | | −13.61 | 1.04 | |
| 1290 | | −7.42 | | −7.86 | | −14.87 |
| 95-3p | | −4.17 | | −3.80 | | −3.85 |
| 375-3p | | −2.11 | | −2.56 | | −1.69 |
| 99a-5p | | −2.55 | | −2.79 | | −3.21 |
| 125b-5p | | −2.52 | | −2.65 | | −2.49 |
| 642a-5p | | −4.76 | | −1.30 | | −6.19 |
| 92a-1-5p | | −4.22 | | −2.61 | | −3.98 |
| 362-5p | | −4.64 | | −17.87 | | −2.60 |
| 342-3p | | −2.48 | | −2.75 | | −2.16 |
| 196a-5p | | −2.89 | | −2.09 | | −1.82 |
| 155-5p | | −2.10 | | −1.74 | | −1.76 |
| 409-3p | 3.20 | | 2.70 | | 1.74 | |
| 744-5p | 2.27 | | 2.20 | | 2.28 | |
| 223-3p | | −1.06 | 2.13 | | 1.34 | |
| 584-5p | 2.90 | | 1.50 | | 1.49 | |
| 340-5p | 2.56 | | | −3.28 | 1.64 | |
| 421 | 2.08 | | 1.44 | | 1.56 | |
| 486-5p | | −1.06 | 1.08 | | | −1.08 |
| 126-3p | | −1.90 | | −1.70 | | −1.44 |
| 320c | 1.16 | | | −1.69 | | −2.22 |
| 320b | 1.21 | | | −1.67 | | −2.03 |
| let-7d-3p | 1.11 | | | −1.45 | | −1.49 |
| 27b-3p | 1.43 | | 1.51 | | 1.43 | |
| 148b-3p | 2.05 | | | −1.09 | 1.48 | |
| 143-3p | 1.04 | | | −22.16 | 1.60 | |
| 326 | 2.10 | | 1.51 | | | −1.03 |
| 335-5p | 2.60 | | 1.01 | | 1.28 | |
| 25-3p | | −1.19 | 1.68 | | 1.09 | |
| 153-3p | | −1.27 | | −1.48 | 1.46 | |
| 19b-3p | | −1.68 | 2.00 | | 1.18 | |
| let-7c-5p | | −1.17 | | −1.14 | 1.04 | |
| Cq-652-3p | no data | | 1.95 | | no data | |
| Cq-376c-3p | no data | | 2.52 | | no data | |
| Cq-154-5p | no data | | 1.61 | | no data | |
| Cq-376a-3p | no data | | | −5.26 | no data | |
| Cq-30d-5p | no data | | 2.10 | | no data | |
| Cq-381-3p | no data | | 2.46 | | no data | |

| | MCI/Control | | Disease Progression from MCI to AD | | | |
|---|---|---|---|---|---|---|
| | Cohort 2 | | | | Cohort 2 | |
| | 27 MCI (21 + 6)/ | | Cohort 1 | | 27 MCI (21 + 6)/ | |
| | 182 Control (82 + 100) | | 39 MCI 39 AD | | 87 AD (62 + 25) | |
| | up | down | up | down | up | down |
| miRNA | regulated | regulated | regulated | regulated | regulated | regulated |
| 151a-3p | 2.40 | | | −1.01 | | −1.13 |
| 151a-5p | 3.38 | | | −1.81 | | −1.81 |
| 146a-5p | 1.70 | | 1.38 | | | −1.18 |
| 4454 | | −7.47 | 2.11 | | 3.23 | |
| 10b-5p | | −11.32 | 1.36 | | 3.40 | |
| 199a-3p | 2.47 | | | −1.07 | | −1.41 |
| 199a-5p | 5.73 | | | −1.35 | | −1.96 |
| 29b-3p | | −1.36 | | −1.56 | 1.36 | |
| 126-5p | | −1.52 | 1.06 | | 1.14 | |
| 150-5p | | −8.77 | 1.07 | | 2.29 | |
| 107 | 3.62 | | | −2.22 | | −2.07 |
| let-7g-5p | | −15.98 | | −1.37 | 1.17 | |
| 1290 | | −24.02 | 2.00 | | 3.06 | |
| 95-3p | | −7.61 | | −1.08 | 2.00 | |
| 375-3p | | −4.11 | | −1.25 | 1.61 | |
| 99a-5p | | −5.58 | 1.26 | | 2.00 | |
| 125b-5p | | −4.22 | | −1.01 | 1.59 | |
| 642a-5p | | −7.25 | 1.30 | | 5.57 | |
| 92a-1-5p | | −3.33 | | −1.06 | 1.28 | |
| 362-5p | | −37.96 | | −1.78 | 2.12 | |
| 342-3p | | −3.24 | | −1.15 | 1.18 | |
| 196a-5p | | −2.70 | | −1.59 | 1.29 | |
| 155-5p | | −1.38 | | −1.20 | | −1.26 |
| 409-3p | 4.17 | | 1.84 | | | −1.55 |
| 744-5p | 3.88 | | 1.00 | | | −1.76 |
| 223-3p | 2.47 | | | −1.42 | | −1.16 |
| 584-5p | 1.44 | | 1.95 | | 1.04 | |
| 340-5p | | −2.62 | 1.56 | | | −1.25 |
| 421 | 1.24 | | 1.33 | | 1.16 | |
| 486-5p | | −2.64 | 1.02 | | 2.84 | |
| 126-3p | | −1.37 | | −1.32 | | −1.24 |
| 320c | | −2.68 | 2.58 | | 1.59 | |
| 320b | | −2.43 | 2.46 | | 1.46 | |
| let-7d-3p | | −1.66 | 1.65 | | 1.14 | |
| 27b-3p | 1.85 | | | −1.01 | | −1.23 |
| 148b-3 p | 1.55 | | 1.39 | | | −1.69 |
| 143-3p | | −29.07 | | −1.54 | 1.31 | |
| 326 | 1.61 | | 2.15 | | | −1.06 |
| 335-5p | | −1.22 | 2.03 | | 1.24 | |
| 25-3p | | −1.14 | | −1.29 | 1.91 | |
| 153-3p | | −1.24 | | −1.86 | | −1.19 |
| 19b-3p | 1.33 | | | −1.98 | 1.51 | |
| let-7c-5p | 1.30 | | | −1.21 | | −1.47 |
| Cq-652-3p | 2.75 | | no data | | | −1.41 |
| Cq-376c-3p | 2.86 | | no data | | | −1.14 |
| Cq-154-5p | 1.63 | | no data | | | −1.01 |
| Cq-376a-3p | | −7.97 | no data | | 1.52 | |
| Cq-30d-5p | 2.35 | | no data | | | −1.12 |
| Cq-381-3p | 3.99 | | no data | | | −1.62 |

### Classification Algorithms

The data from Table 14 was used to create a prediction model for the assessment of accuracy, sensitivity, and specificity. The data were subject to a ten-fold cross-validation, multinomial, relaxed fit, glmnet model in R-studio using the several libraries (data.table, glmnet, knitr, and caret), softmax function, deltaCq values from 50 miRNA as the X variable and class identification as the y variable, seedset of 42, mean square error as the measure, z-score data standardization, lasso variable selection, with a convergence threshold of 1 e−7, and 1 e7 maximum iterations. An algorithm was created, using the techniques described herein, that selected, through a feature selection or variable selection, which of the miRNA examined through qPCR contributed the most to separating AD, MCI, and healthy controls. Using the classification algorithm, a model was created based on the data from hundreds of individual patients with known disease classification. Coefficients were assigned to each of the miRNA based on the relative contribution of each miRNA to the model. Using this model, the combined qPCR data from a single individual can then be compared to the model to determine a disease classification prediction for that individual.

The Cross-Validation glmnet Model in R data is provided in Table 15 below.

TABLE 15

| Class | AD (%) | Control (%) | MCI (%) |
|---|---|---|---|
| Sensitivity | 79 | 91 | 79 |
| Specificity | 90 | 90 | 96 |
| Positive | 78 | 91 | 79 |
| Predictive | | | |
| Value | | | |
| Negative | 91 | 90 | 96 |
| Predictive | | | |
| Value | | | |
| Balanced | 84 | 91 | 88 |
| Accuracy | | | |
| AUC | 80 | 92 | 91 |

Accuracy: 0.85
95% CI: (0.82, 0.89)
P-Value [Acc > NIR]: <2e−16
Kappa: 0.75
Mcnemar's Test P-Value: 0.83

### Example 4—Creating a Predictive Model and System to Diagnose AD and/or MCI

Mild cognitive impairment (MCI) is generally considered a transitional state between normal cognitive aging and Alzheimer's disease (AD). However, the line at which an individual crosses over from MCI to AD is murky, and reversal from MCI back to a healthy state has been calculated in 16% of diagnosed patients (Koepsell T D, Monsell S E, “Reversion from mild cognitive impairment to normal or near-normal cognition: risk factors and prognosis,” Neurology, 2012 Oct. 9; 79 (15): 1591-8. doi: 10.1212/WNL.0b013 e31826 e26b7. Epub 2012 Sep. 26. PMID: 23019264; PMCID: PMC3475624). Furthermore, the underlying pathology and risk factors leading to MCI and from MCI to AD is controversial. In any given year, the probability of developing AD after the initial diagnosis of MCI is about 15% (Wang Y, Li M, Haughton D, Kazis L E, “Transition of mild cognitive impairment to Alzheimer's disease: Medications as modifiable risk factors,” PLoS ONE 19 (8): e0306270, 2024; https://doi.org/10.1371/journal.pone.0306270).

This example identifies a miRNA signature found in blood plasma that aids in diagnosing and separating MCI and AD, with the aid of a multinomial logistic regression model to create the miRNA signature model and system. Venous blood samples from individuals identified by physicians with MCI (n=92) and AD (n=185), as well as from individuals without known cognitive impairment (n=428) were collected in K2 EDTA tubes and centrifuged to separate the blood plasma. Extraction of neural-enriched extra-cellular vesicles was conducted from a subset of blood samples using immunoaffinity, as described in Example 1 above, in U.S. Patent Application Publication No. 2021/0164051, and Banack et al., “An miRNA fingerprint using neural-enriched extracellular vesicles from blood plasma: towards a biomarker for amyotrophic lateral sclerosis/motor neuron disease,” Open Biol., Vol. 10, pp 1-12 (2020), the disclosure of which is incorporated by reference herein in its entirety. Next generation sequencing was performed to identify differentially expressed miRNA between groups (Banack et al. 2020). RNA was then extracted directly from the blood plasma of a large number of individuals. Blood plasma includes RNA circulating in the blood plus RNA found in extracellular vesicles within the blood and the RNA extraction process collects both simultaneously. Following cDNA synthesis, a qPCR analysis was carried out on the 49 miRNA targets listed in Table 14 above as a more precise measurement of miRNA abundance, using the same techniques discussed in Example 3. The raw data from qPCR was normalized using the geometric mean of 3 stable endogenous miRNA to account for RNA loading.

The normalized qPCR data from all 705 individuals were then used to conduct a relaxed fit, no penalty, multinomial logistic regression (glmnet) in R-studio with four libraries: data.table, glmnet, knitr, and caret. The model used a lasso feature selection which kept out-of-fold predictions and included the following parameters: seed set of 42, multinomial family, grouped type, mse measure, Z-score standardization, intercept included, lambda sequence (exp(seq(−4, −1, length.out=1000), 20 nfolds, convergence threshold of 1 e−6, 1 e7 maximum iterations.

The fit model calculated an AUC score of 0.84, which is a measure of the entire two dimensional area under the Receiver Operation Characteristic curve. The mean standard error was 0.08. Overall accuracy was 0.84 (95% CI: 0.812, 0.8674) and a Kappa score of 0.70. The classification sensitivity, specificity, and model predictive values were high (Table 16).

TABLE 16

| Class | AD (%) | Control (%) | MCI (%) |
|---|---|---|---|
| Sensitivity | 76.2 | 91.8 | 64.1 |
| Specificity | 92.3 | 83 | 96 |
| Positive Predictive | 77.9 | 89.3 | 70.2 |
| Value | | | |
| Negative Predictive | 91.6 | 86.8 | 94.7 |
| Value | | | |
| Prevalence | 26.2 | 60.7 | 13.1 |
| Detection Rate | 20 | 55.7 | 8.4 |
| Detection | 25.7 | 62.4 | 11.9 |
| Prevalence | | | |
| Balanced Accuracy | 84.3 | 87.4 | 80 |

Carrying out the above analysis, 16 miRNAs were identified in the model generated to differentiate between AD, MCI, and controls, each with a coefficient identifying the contribution of the miRNA to the model, as shown in Table 17 below. The model used a Lasso feature selection that iteratively determined which miRNA add more to distinguishing the respective disease states and eliminated miRNA that added less. The Lasso feature selection selected the 16 miRNA, which were determined to have the greatest discriminatory power and provided the requisite predictive accuracy. Table 17 provides the general linear model coefficients for Alzheimer's Disease, Mild Cognitive Impairment, and healthy controls.

TABLE 17

| miRNA | Class AD | Class Control | Class MCI |
|---|---|---|---|
| (Intercept) | 7.7818 | −0.3909 | −7.3909 |
| 151a-3p | 0.3876 | 0.7540 | −1.1416 |
| 199a-5p | −0.0529 | 0.1793 | −0.1263 |
| 150-5p | 0.6419 | −0.4377 | −0.2042 |
| 375-3p | 0.1726 | 0.3627 | −0.5352 |
| 99a-5p | −0.3193 | −1.2245 | 1.5439 |
| 642a-5p | −0.4019 | 0.1991 | 0.2028 |
| 92a-1-5p | −0.2409 | 0.0424 | 0.1986 |
| 223-3p | −0.6022 | 0.0793 | 0.5229 |
| 421 | −0.2634 | 0.1374 | 0.1260 |
| 320b | −0.6531 | −0.0350 | 0.6881 |
| 335-5p | 0.1225 | −0.3280 | 0.2055 |
| 153-3p | −0.1706 | 0.1432 | 0.0274 |
| 19b-3p | 0.0911 | 0.5968 | −0.6878 |
| 652-3p | 0.0385 | −0.1141 | 0.0757 |
| 30d-5p | −0.1829 | 0.0420 | 0.1409 |

It is interesting that from the individuals with diagnosed MCI, 16% were predicted to have AD which is consistent with the expected transition rate (Wang et al. 2024). In addition, 20% of diagnosed MCI patients were classified with the control group and this also represents approximately the number that is expected to revert back to normal (Koepsell et al. 2012). The sensitivity of the model, or its ability to predict AD, which is a later disease stage with more severe loss of cognitive function, was higher (76%) and the ability of the model to exclude AD as a diagnosis was 92%. Those skilled in the art recognize that a glm model is a complicated model of logarithmic odds. In the binomial glm model of just (AD and MCI), a one unit change in the miRNA delta Cq multiplies the odds of the person having AD by eB1 (Equation 1). Roughly speaking, a higher coefficient adds more to the model than a lower coefficient. A positive coefficient number in the Class AD column for each respective miRNA (e.g., miR-151a-3p has a Class AD coefficient of 0.3876) indicates that its presence makes it more likely that the person has AD. In contrast, a negative number (e.g., miR-199a-5p has a Class AD coefficient of −0.0529) makes it less likely (when the miRNA is abundant) that the person has AD. The sum of the coefficients in each row for each respective miRNA therefore results in a value of zero. The combined effect of all the miRNA coefficients in each column makes the final prediction.

Carrying out the above analysis, 11 miRNAs were identified in the model (using the Lasso feature selection method) that was used to differentiate between AD and MCI, each with a coefficient identifying the contribution of the miRNA to the model, as shown in Table 8 below. Using the same parameters in a multinomial logistic regression model of 185 AD and 92 MCI patients, the model was capable of predicting AD with 90% sensitivity and MCI with 78% sensitivity. The model had an AUC score of 0.93 and mean standard error of 0.11. Table 18 provides the general linear model coefficients for differentiating Alzheimer's Disease from Mild Cognitive Impairment.

TABLE 18

| miRNA | Class AD | Class MCI |
|---|---|---|
| (Intercept) | 1.2565 | −1.2565 |
| 4454 | −0.6488 | 0.6488 |
| 95-3p | 0.3241 | −0.3241 |
| 375-3p | 0.3554 | −0.3554 |
| 642a-5p | −0.3370 | 0.3370 |
| 92a-1-5p | −0.0570 | 0.0570 |
| 196a-5p | 0.2718 | −0.2718 |
| 223-3p | 0.0741 | −0.0741 |
| 421 | −0.1832 | 0.1832 |
| 320b | −0.9178 | 0.9178 |
| 335-5p | 0.0712 | −0.0712 |
| 30d-5p | −0.0781 | 0.0781 |

As expected, this model was more accurate for AD since the underlying disease etiology is less variable than MCI. With the model in place, the combined qPCR data from a single individual can then be compared to the model to determine a disease classification prediction for that individual.

The model, which is a predictive algorithm, may be created using a large number of individuals with a known disease status, and can and likely will be repeatedly updated as more data is fed into the model. Thus, the miRNA and their respective coefficients may change over time (e.g., the identified miRNA and their respective coefficients provided in Tables 7 and 8 above may change over time). But once the algorithm is created as described above in Examples 1 and 2, individuals of unknown disease status can be tested using the model, which will provide a prediction of the likelihood that the individual has or does not have the disease. This can be accomplished by first drawing a blood sample, centrifuging the blood to retrieve the blood plasma fraction, extracting the RNA, creating cDNA, and quantifying the abundance of each of the miRNAs identified in the model using qPCR techniques. The raw cycle quantitation (Cq) numbers can then be normalized by the geomean of three endogenous miRNAs as described above, thus generating a delta Cq value for each miRNA, using the techniques discussed in the examples hereinabove (see Banack et al., (2024), and Vandesompele et al., (2002)). These deltaCq values are then entered into the model (e.g., predictive algorithm), and each miRNA value contributes to the prediction of disease state incrementally. In the multinomial model, a positive coefficient means that a one-unit increase in the value for that specific miRNA increases the log-odds of the individual having disease A, and decreases the log-odds of the person having disease B and C, relative to the overall average. When the coefficient is negative, the value for that specific miRNA decreases the log-odds of the individual having disease A. The combination of log-odds for each miRNA in the model determines the final prediction that a person does or does not have the disease. The model adds up the likelihood that the person has AD by adding up each of the log odds ratios. It then generates a prediction number that indicates that the person is predicted to have AD with 98% certainty, for example.

### Example 5 Developing an miRNA Fingerprint for any Motor Neuron Disease or Neurodegenerative Disorder

Plasma samples were collected in K2 EDTA tubes from a total of 1,142 individuals, including 318 normal controls (healthy subjects) and 824 subjects with neurological impairment covering a variety of neurological diseases (ALS, MS, PD, PLS, AD, MCI, Lyme disease), and were separated into cohort groups. Normal, healthy control subjects had a mean age of 54 (min age=41, max age=71). All blood samples were collected in K2 EDTA tubes and samples blinded before being processed and analyzed. Every patient sample was from a unique individual with no overlap between cohorts.

### RNA Extraction

RNA extraction was carried out according to the manufacturer's instructions in Qiagen miRNeasy Serum/Plasma Kit (Qiagen, cat. no. 217184). To 200 μL plasma, were added 1 ml QIAzol lysis buffer spiked with 1 μL of UniSp2, UniSp4, and UniSp5 available as part of the RNA Spike-in Kit, For RT, (Qiagen, cat. no. 339390). Spike-ins were to monitor RNA extraction efficiency.

### CDNA Synthesis

cDNA was synthesized using the miRCURY LNA RT Kit (Qiagen, cat. no. 339340) according to the manufacturer's instructions, but with the modification of 4 μL total RNA per reaction, rather than 2 μL as recommended by the manufacturer. This was because it was previously determined that 4 μL returns a more robust signal (Dunlop et al, 2021). Included was the addition of 1 μL of a spike-in control mix containing UniSp6 and Caenorhabditis elegans cel-miR-39-3p to each cDNA reaction to monitor cDNA synthesis (reverse transcription) efficiency. UniSp6 was provided in the miRCURY LNA RT kit, and cel-miR-39-3p was included in the RNA Spike-in Kit, for RT. Each 10 μL cDNA synthesis reaction was conducted in duplicate and pooled to create a total of 20 μL cDNA. cDNA was aliquoted into aliquots to avoid multiple freeze/thaw cycles, prior to storage at −20 °C.

Real-Time Quantitative PCR (qPCR) Quality Control

Following cDNA synthesis, quality control (QC) qPCR was carried out for every sample on ten QC miRNA targets. To determine the success of the RNA extraction step, raw Cqs for the spike-ins UniSp2, UniSp4, and UniSp5 were obtained. A “pass” was given if the standard deviation (SD) for all individual samples varied by less than two SD from the mean raw CQ for all samples in amplicon group. To determine if reverse transcription (RT) (cDNA synthesis) proceeded without inhibition, the spike-ins UniSp6 and cel-miR-39-3p were obtained. A “pass” was given if the standard deviation (SD) for all individual samples varied by less than two SD from the mean raw CQ for all samples in amplicon group.

“Sample signal” was used to estimate the amount of cDNA in each sample and determine whether samples were concentrated enough for downstream analysis. For determination of sample signal, five endogenous miRNAs: miR-142-3p, miR-451a, miR-23a-3p, miR-30c-5p, and miR-103a-3p were measured using qPCR. Matias-Garcia P R, et al., “Impact of long-term storage and freeze-thawing on eight circulating microRNAs in plasma samples,” PLOS ONE, 15(1):e0227648 (2020). Samples that returned raw Cqs≤34 were given a “pass.”

qPCR of miRNA Using SYBR Green

qPCR for all QC, target, and reference miRNAs was conducted using Qiagen miRCURY LNA miRNA SYBR PCR Assays (cat. no. 339306, and the miRCURY LNA™ SYBR Green PCR Kit (Qiagen, cat. no. 339347) according to the manufacturer's instructions. cDNA was diluted 1/30 into nuclease-free water and 3 μL aliquoted into each 10 μL qPCR reaction. The pipetting of master mix (7 μL) and samples (3 μL) into 384-well plates was automated using the Opentrons OT-2 liquid-handling robot using a custom protocol written in Python. qPCR was conducted on the Bio-Rad CFX Opus 384 in 384-well plates (Bio-Rad Hard Shell PCR Plates, thin-wall, cat. no. HSP3805). Data in Bio-Rad CFX Maestro version 2.3 were acquired after 40 cycles. Raw Cqs were processed in web-based LinRegPCR to obtain efficiency-corrected Cqs ((Untergasser A. et al., Web-based LinRegPCR: application for the visualization and analysis of (RT)-qPCR amplification and melting data. BMC Bioinformatics; 22:398. 2021). Primer amplification efficiencies varied by less than 5%, meaning efficiency corrections were not required to compare results across primers. Each plate contained triplicate inter-plate calibrators (TATAA IPC, TATAA Biocenter, cat. no. IPC250S) pipetted into the same wells on every plate, to enable comparison of samples with prior cohorts. A melt curve and triplicate no-template controls in each assay were included to check for primer specificity and any nonspecific amplification.

### Sample Normalization (ΔCq)

Disease samples were normalized to healthy controls for RNA loading and reaction inhibition using the geometric mean of three stable endogenous miRNA following the protocol of Vandesompele, (Vandesompele J, et al., “Accurate normalization of real-time quantitative RT-PCR data by geometric averaging of multiple internal control genes,” Genome Biol., 3(7):1-12 (2002)).

### Calculations

Sensitivity and specificity were evaluated using normalized ΔCq values. Sensitivity is a measure of the ability of a screening test to positively classify someone who has the disease. It is calculated as a percent identification of true positive cases in the dataset divided by the total number of individuals with the disease, as defined by a gold standard method of disease identification, described in, for example, Trevethan R, “Sensitivity, Specificity, and Predictive Values: Foundations, Pliabilities, and Pitfalls in Research and Practice,” Front Public Health, 5:30 (2017). Specificity is a measure of the ability of a screening test to accurately classify someone who does not have the disease, comparing true negative cases to the total number of individuals without the disease in the dataset. Also important for biomarker evaluation is a measure of positive predictive value (PPV) for the test, which compares true positive cases with false positive identification. The negative predictive value (NPV) of a test is the number of true negative cases identified by the test relative to false negative identification (see Trevethan above). Area under the cure (auc) and mean standard error (mse) may also be helpful in determining the accuracy of the model.

The calculation to determine if a person has or is at risk of developing a motor or neurological disorder was performed a logistic regression algorithms. A logistic regression was carried out on non-IPC corrected, ΔCq data with a Lasso feature selection using the following model parameters: seed set 42, multinomial family, MSE measure, Z-score standardization, relaxed glm fit without penalty, lambda sequence (−4, −1, length.out=1000, min), 40 nfolds, 1 e⁻⁷ convergence threshold, and 1 e⁵ maximum iterations. Logistic regression machine learning algorithms also are well known in the art and are used to accomplish binary classification tasks by predicting the probability of an outcome, event, or observation. Those skilled in the art are capable of using random forest machine learning algorithms, logistic regression algorithms, or other classification techniques to develop software or a protocol for carrying out the method of identifying a subject who has, or is at risk of developing a motor neuron disease or other neurological disease using the presence or absence of two more of miRNA selected from miR-151a-3p, miR-199a-5p, miR-150-5p, miR-1290, miR-99a-5p, miR-342-3p, miR-223-3p, miR-652-3p. The software can be provided with one or more kit(s) described herein the first time a kit is purchased, and loaded onto a computing device integral to, incorporated into, or in communication with a device for conducting qPCR.

In accordance with this example, which carried out testing on 1142 individuals including 318 normal, healthy controls and 824 patients with neurological impairment, 8 miRNAs were identified, which, together can differentiate individuals who are healthy from those with neurological impairment with high accuracy. The 8 miRNAs are listed in Table 2 above, and include miR-151a-3p, miR-199a-5p, miR-150-5p, miR-1290, miR-99a-5p, miR-342-3p, miR-223-3p, miR-652-3p. The Cross-Validation glmnet Model in R data is provided in Table 19 below.

TABLE 19

| Class | Median (95% Confidence Interval) |
|---|---|
| Sensitivity | 0.93 (0.90-0.96) |
| Specificity | 0.97 (0.96-0.98) |
| Positive | 0.93 (0.90-0.95) |
| Predictive | |
| Value | |
| Negative | 0.97 (0.96-0.98) |
| Predictive | |
| Value | |
| Balanced | 0.96 (0.95-0.97) |
| Accuracy | |
| AUC | 0.99 (0.98-0.99) |
| mse | 0.03 (0.02-0.04) |

### Example 6 Use of the Model on ALS Subjects

A comparison of models indicates high classification accuracy across models, as noted below:

Models: LogisticReg, ElasticNet, RandomForest, XGBoost
Number of resamples: 50

| F1 | Min. | 1st Qu. | Median | Mean | 3rd. Qu. | Max. | NA's |
|---|---|---|---|---|---|---|---|
| LogisticReg | 0.8907563 | 0.9313267 | 0.9464286 | 0.9472368 | 0.9649123 | 0.9913043 | 0 |
| ElasticNet | 0.9122807 | [symbol] | 0.9561370 | 0.9566469 | 0.9655172 | 0.9911504 | 0 |
| RandomForest | 0.9310345 | 0.9672572 | 0.9819805 | 0.9771202 | 0.9911504 | 1.0000000 | 0 |
| XGBoost | 0.9482759 | 0.9730926 | 0.9824561 | 0.9798536 | 0.9911504 | 1.0000000 | 0 |
| Precision | | | | | | | |
| LogisticReg | 0.8412698 | [symbol] | [symbol] | 0.9423346 | 0.9645933 | 1 | 0 |
| ElasticNet | [symbol] | 0.9310345 | 0.9491525 | 0.9491327 | 0.9647556 | 1 | 0 |
| RandomForest | 0.9000000 | 0.9642857 | 0.9821429 | 0.9741633 | 1.0000000 | 1 | 0 |
| XGBoost | 0.9193548 | 0.9649123 | 0.9824561 | 0.9780942 | 1.0000000 | 1 | 0 |
| Recall | | | | | | | |
| LogisticReg | 0.8596491 | 0.9298246 | 0.9473684 | 0.9532206 | 0.9821429 | 1 | 0 |
| ElasticNet | 0.8928571 | 0.9473684 | 0.9642857 | 0.9648997 | 0.9823778 | 1 | 0 |
| RandomForest | 0.9464286 | 0.9649123 | 0.9821429 | 0.9804825 | 1.0000000 | 1 | 0 |
| XGBoost | 0.9285714 | 0.9649123 | 0.9824561 | 0.9819361 | 1.0000000 | 1 | 0 |
| ROC | | | | | | | |
| LogisticReg | 0.9371867 | 0.9847526 | 0.9895930 | 0.9883641 | 0.9950523 | 0.9990602 | 0 |
| ElasticNet | 0.9765038 | 0.9864574 | 0.9931672 | 0.9911869 | 0.9958682 | 1.0000000 | 0 |
| RandomForest | 0.9786022 | 0.9945959 | 0.9981365 | 0.9962755 | 0.9993924 | 1.0000000 | 0 |
| XGBoost | 0.9839685 | 0.9942043 | [symbol] | 0.9959846 | 0.9993844 | 1.0000000 | 0 |
| Sens | | | | | | | |
| LogisticReg | [symbol] | 0.9298246 | 0.9473684 | 0.9532206 | 0.9821429 | 1 | 0 |
| ElasticNet | 0.8928571 | 0.9473684 | 0.9642857 | 0.9648997 | [symbol] | 1 | 0 |
| RandomForest | 0.9464286 | [symbol] | 0.9821429 | [symbol] | 1.0000000 | 1 | 0 |
| XGBoost | [symbol] | [symbol] | 0.9824561 | 0.9819361 | 1.0000000 | 1 | 0 |
| Spec | | | | | | | |
| LogisticReg | [symbol] | 0.9137931 | 0.9473684 | 0.9414035 | [symbol] | 1 | 0 |
| ElasticNet | 0.8947368 | [symbol] | 0.9473684 | [symbol] | [symbol] | 1 | 0 |
| RandomForest | 0.8947368 | 0.9649123 | [symbol] | 0.9738234 | 1.0000000 | 1 | 0 |
| XGBoost | 0.9137931 | 0.9649123 | [symbol] | [symbol] | 1.0000000 | 1 | 0 |

[symbol]

indicates data missing or illegible when filed

The model discussed in the examples above was used to analyze data from known subjects with ALS, and known healthy subjects. A number of samples from subjects known to have ALS, or having previously been diagnosed with ALS, and a number of samples from subjects known not to have ALS were obtained and analyzed as described in the examples of above. The Cq, delta Cq values, and coefficients for the miRNA listed in Table 20 below were determined using the model.

TABLE 20

| miRNA | ALS | Control |
|---|---|---|
| (Intercept) | −8.5997 | 8.5997 |
| 199a-5p | −0.6212 | 0.6212 |
| 126-5p | 0.2511 | −0.2511 |
| 150-5p | 0.5237 | −0.5237 |
| 99a-5p | 1.1806 | −1.1806 |
| 223-3p | −0.8449 | 0.8449 |
| 335-5p | 0.5372 | −0.5372 |
| let-7g-5p | 0.3496 | −0.3496 |
| let-7c-5p | −0.2842 | 0.2842 |
| 486-5p | −0.7630 | 0.7630 |
| 199a-3p | 0.4431 | −0.4431 |
| 155-5p | −0.5309 | 0.5309 |

This model had an overall accuracy of 94% (95% CI: 0.9248, 0.9533). The sensitivity of the model, or ability to correctly classify ALS patients, was 96% and the specificity of the model, or ability of the model to correct classify non-ALS patients was 93%.

### Discussion of Results

The above examples can be repeated using samples from healthy subjects and compared to subjects known to have or previously diagnosed with additional motor neuron diseases and/or neurodegenerative disorders, such as MS, HD, CJD, PSP, LBD, MSA, FTD, and others, to provide lists of miRNA suitable for diagnosis and disease and/or disorder differentiation for multiple diseases and/or disorders. The aforementioned lists can be incorporated into a suitable final miRNA panel that can be used to diagnose (and monitor treatment of) a motor neuron disease and/or neurodegenerative disorder in general, or any one of a variety of motor neuron diseases and/neurodegenerative disorders using a single blood draw or minimal number of blood draws.

Patients having or suspected of having a motor neuron disease and/or neurodegenerative disorder, and the neurologists that diagnose them need a suitable diagnostic technique to diagnose the disease and/or disorder early and begin treatment, if needed. Despite worldwide awareness of the motor neuron diseases and/or neurodegenerative disorders discussed herein, and a plethora of scientific research, patients are still faced with a significant diagnosis delay. The rapid progression of the disease increases the need for a quick and accurate diagnostic test from easily obtained biosamples. Adding a blood-based diagnostic test to confirm suspected motor neuron disease and/or neurodegenerative disorder would help physicians and benefit patients. The results presented herein have demonstrated the robust, reproducible, sensitive, and accurate ability of a final miRNA panel that can be used to diagnose a motor neuron disease and/or neurodegenerative disorder in general, or one or more identified motor neuron diseases and/or neurodegenerative disorders, including ALS, PLS, AD, MCI, PD, MS, HD, CJD, LBD, MSA, FTD, and/or PSP, to test subjects to rule out a future general or ALS, PLS, AD, MCI, PD, MS, HD, CJD, LBD, MSA, FTD, and/or PSP diagnosis, and to differentiate between ALS, PLS, AD, MCI, PD, MS, HD, CJD, LBD, MSA, FTD, and/or PSP, and other neurological diseases, as well as healthy subjects. This final miRNA panel (or biomarker panel) can be utilized as a simple and rapid secondary measure of disease and/or disorder diagnosis following a physician's clinical evaluation. The enhanced diagnostic confidence provided by a biomarker would enable physicians to diagnosis a motor neuron disease and/or neurodegenerative disorder in general, or to specifically diagnose ALS, PLS, AD, MCI, PD, MS, HD, CJD, LBD, MSA, FTD, and/or PSP patients, as well as identify subjects at risk of developing a motor neuron disease and/or neurodegenerative disorder, or developing ALS, PLS, AD, MCI, PD, MS, HD, CJD, LBD, MSA, FTD, and/or PSP, at an earlier stage. This in turn would benefit patients by providing an opportunity for earlier use of disease modifying a motor neuron disease and/or neurodegenerative disorder in general, or ALS, PLS, AD, MCI, PD, MS, HD, CJD, LBD, MSA, FTD, and/or PSP drugs or other therapies.

In order to qualify as a new diagnostic biomarker with Food and Drug Administration (FDA) approval, four criteria must be met: 1) a needs assessment; 2) context of use; 3) benefit/risk; 4) evidence to support qualification. U.S. Department of Health and Human Services. Biomarker Qualification: Evidentiary Framework Guidance for Industry and Staff 2018, available on the FDA website. The high sensitivity, specificity, positive predictive value, and negative predictive value suggests that the benefits of the final miRNA panel (biomarker panel) in conjunction with clinical evaluation would outweigh any risk, particularly given the rapid rate of motor neuron disease and/or neurodegenerative disorder progression. Further evidence to support qualification includes consistent fold-change calculations and highly different fold-regulation between comparison groups as noted by a Mann Whitney analyses.

miRNA represent good candidate biomarker targets due to their regulatory roles in essential cell functions. Many miRNA have been identified as dysregulated in various motor neuron diseases and/or neurodegenerative disorders with on-going research investigating their biomarker potential.

Disparity between studies is a concern in miRNA research and can be attributed largely to tissues, methods, sample size, and research focus with a resulting multiplicity of fine detail that can alter the end results between studies. See, e.g., Koshiol J, et al., “Strengths and limitations of laboratory procedures for microRNA detection,” Cancer Epidemiol Biomarkers Prev., 19(4):907-911 (2010); and Shademan B, et al., “MicroRNAs as Targets for Cancer Diagnosis: Interests and Limitations,” Adv Pharm Bull., 13(3):435 (2023). Reproducibility in miRNA studies can be impacted by: a) pre-analytical variables including sample collection and processing procedures; b) RNA isolation, quantification, and handling methods; c) limited or lack of internal quality control measures; d) data acquisition, processing, and statistical analysis variability. Lakkisto P, et al., “Development of circulating microRNA-based biomarkers for medical decision-making: a friendly reminder of what should NOT be done,” Crit Rev Clin Lab Sci., 60(2):141-152 (2023). Furthermore, few studies have been adequately validated using large samples sizes of unique patient cohorts.

The embodiments disclosed herein provide a method to enhance reproducible miRNA discovery intended for clinical applications. This robust final miRNA panel (“biomarker panel”) for a motor neuron disease and/or neurodegenerative disorder in general, or specifically ALS, PLS, AD, MCI, PD, MS, HD, CJD, LBD, MSA, FTD, and/or PSP was identified through a series of steps, and then surprisingly discovering that the biomarker panel can be used with a simple blood draw and provide a more accurate diagnosis of a motor neuron disease and/or neurodegenerative disorder in general, or specifically ALS, PLS, AD, MCI, PD, MS, HD, CJD, LBD, MSA, FTD, and/or PSP. The current cohort of samples represent blood plasma collected from several sources using generally accepted plasma collection protocols but without standardization. These samples are thus more heterogeneous in nature, varied in storage duration, and diagnosing neurologist. This diversity in pre-analytical sample sources suggests that the final miRNA panel (biomarker panel) was not sensitive to specific blood plasma collection protocols, an assertion that is consistent with the inventors' prior studies, and increases the likelihood of future reproducibility.

In comparison to all miRNA found to be dysregulated in a motor neuron disease and/or neurodegenerative disorder in general, or specifically ALS, PLS, AD, MCI, PD, MS, HD, CJD, LBD, MSA, FTD, and/or PSP, the final miRNA panel identified herein that has been found to be valuable in various combinations, are a small subset of the those identified. However, the final miRNA panel has important links to a motor neuron disease and/or neurodegenerative disorder in general, or specifically ALS, PLS, AD, MCI, PD, MS, HD, CJD, LBD, MSA, FTD, and/or PSP biological pathways. The precise function of each miRNA and target pathways related to disease and/or disorder onset and progression are not yet clear. Prior research suggests that the final miRNA panel affects biological processes consistent with motor neuron diseases and/or neurodegenerative disorders affecting such biological processes as oxidative stress, cell viability, motor neuron loss, synaptic transmission, neuron regeneration, neural inflammation, and more. However, many miRNsA have multiple targets; including several miRNAs from different pathways can increase the robust nature and potential specificity of the test.

In contrast to known diagnostic methods, the present inventors have discovered the ability of this final miRNA panel (biomarker panel) for a motor neuron disease and/or neurodegenerative disorder in general, or specifically ALS, PLS, AD, MCI, PD, MS, HD, CJD, LBD, MSA, FTD, and/or PSP diagnosis with high sensitivity and accuracy. The final miRNA panel described herein provides evidence of a strong relationship between this diagnostic biomarker and the gold standard method of clinical disease diagnosis. The analytical performance for NEE is good with important predictive value, while the analytical performance for blood-based detection without exosome isolation is beneficial. The use of independent cohort groups in the machine learning algorithm, with one cohort used for training and another cohort for testing, provides support for the predictive value of the test. The analytical validation of the multi-miRNA fingerprint in this study establishes that the biomarker test is both accurate and reproducible on blood plasma collected using multiple collection protocols. It is not sensitive to variation in standard blood plasma collection, storage, or processing conditions which increases its potential for clinical diagnostic evaluation. Further validation of this biomarker using samples from prospective and longitudinal studies, as well additional neurological controls, would aid its future use and accelerate its introduction into the clinic and clinical trials.

In some aspects, presented herein is one or more kits useful in carrying out a method of identifying a subject who has, or is at risk of developing a motor neuron disease that includes one or more multi-well plates, with each well contains one or more miRNA primers selected from the group consisting of miR-151a-3p, miR-199a-5p, miR-150-5p, miR-1290, miR-99a-5p, miR-342-3p, miR-223-3p, miR-652-3p, and a mechanism to calculate the likelihood of a positive or negative diagnosis, and/or probability of having or not having a neurological disease. In one embodiment, the kit includes two well-plates in which one well plate includes each well containing one or more miRNA primers selected from the list above, and the other well plate includes a Quality Control (QC)/spike-in/calibration plate in which one or more wells contain QC primers, no-template controls, and/or spike-in primers designed to monitor the success of the method. In another embodiment, the kit includes one well-plate in which the wells include one or more miRNA primers selected from the list above, QC primers, no-template controls, and/or spike-in primers designed to monitor the success of the method. In a further embodiment, the mechanism to calculate the likelihood of a positive or negative diagnosis, and/or probability of having or not having a neurological disease includes software configured to carry out the calculation using random forest machine learning algorithms and/or logistic regression algorithms developed from machine learning classification of known disease and healthy control plasma samples. In some embodiments, a subject is a healthy individual tested as a preventative screen to rule out the possibility of being at risk of developing a motor neuron disease and to identify the possibility of onset at an early stage before symptoms arise.

The well-plates useful in the kits described herein may include, for example, qPCR plates of varying sizes, including 48, 96, and 384-well plates. Each of the respective wells can pre-loaded with one or more of the miRNA primers selected from miR-151a-3p, miR-199a-5p, miR-150-5p, miR-1290, miR-99a-5p, miR-342-3p, miR-223-3p, miR-652-3p as well as QC primers, no-template controls, and/or spike-in primers. Particularly preferred QC primers, no-template controls, and/or spike-in primers that may be used in the kit(s) described herein are described in more detail in the examples below. In one embodiment, a kit may contain two 96-well plates suitable for 25 μL reactions in which one of the well-plates contains wells filled with one or more of the miRNA primers selected from miR-151a-3p, miR-199a-5p, miR-150-5p, miR-1290, miR-99a-5p, miR-342-3p, miR-223-3p, miR-652-3p, and the other well-plates contains wells filed with QC primers, no-template controls, and/or spike-in primers. In another embodiment, the kit includes a 384-well plate suitable for 10 μL reactions, in which all of the miRNA primers, QC primers, no-template controls, and/or spike-in primers are included on the same plate. The kits can be designed for use in any analyzer capable of carrying out qPCR analysis on the circulating bodily fluid samples added to the respective well-plates, and indicating a presence and/or amount of one or more of miR-151a-3p, miR-199a-5p, miR-150-5p, miR-1290, miR-99a-5p, miR-342-3p, miR-223-3p, miR-652-3p.

In some embodiments, the baseline amount may be an amount considered ‘normal’ for the particular miRNA (e.g., an average amount for age-matched individuals not diagnosed with the motor neuron disease), or the baseline amount may be a historical reference amount for the particular subject (e.g., a baseline amount that was obtained from a circulating bodily fluid sample derived from the same subject, but at an earlier point in time). Quantitative baseline amounts that are determined contemporaneously (e.g., a reference value that is derived from a pool of samples including the sample being tested) are also contemplated. Accordingly, in some embodiments, methods are provided for monitoring progression of a motor neuron disease in a subject by obtaining a quantitative measured amount for one or more miRNAs present in circulating bodily fluid obtained from a sample and comparing such measured value to a baseline amount. In some embodiments, a difference between an amount of one or more miRNAs from circulating bodily fluid obtained from a subject and a baseline amount that is less than the difference obtained at an earlier point in time indicates that the progression of the disease has diminished. In some embodiments, a difference between an amount of one or more miRNAs from circulating bodily fluid obtained from a subject and a baseline amount that is greater than the difference obtained at an earlier point in time indicates that the progression of the disease has increased.

The kits are particularly suited for automated pipetting using a liquid handling robot (e.g., an Opentrons OT-2 robot) for sample loading into the respective wells in the plate(s) of the respective kit(s) that are preloaded with the reagents discussed above. Operators supply the RNA/cDNA. The kits can be designed for use in any analyzer capable of carrying out qPCR analysis on the circulating bodily fluid samples added to the respective well-plates, and indicating a presence and/or amount of one ore more of the miRNAs selected from miR-151a-3p, miR-199a-5p, miR-150-5p, miR-1290, miR-99a-5p, miR-342-3p, miR-223-3p, miR-652-3p. The method of diagnosis can be carried out using the information generated regarding the presence and/or amount of one ore more of the miRNAs selected from miR-151a-3p, miR-199a-5p, miR-150-5p, miR-1290, miR-99a-5p, miR-342-3p, miR-223-3p, miR-652-3p.

The diagnostic method preferably is carried out on-site using a mechanism for calculating the likelihood of a positive or negative diagnosis, and/or probability of having or not having a neurological disease. A preferred device for conducting the qPCR includes the use of a convention qPCR real time diagnostic system, such as those available as BioRad CFX series, Applied Biosystems, Edvotek, MSE PRO series, Benchmark, Roche, Thermo Scientific, and the like. These devices can be programmed to calculate the likelihood of a positive or negative diagnosis, and/or probability of having or not having a neurological disease, or the data from the qPCR system can be fed to a computing apparatus configured to calculate the likelihood of a positive or negative diagnosis, and/or probability of having or not having a neurological disease.

The calculation may be performed using random forest machine learning algorithms, logistic regression algorithms developed from machine learning classification of known disease and healthy control plasma samples or other classification algorithms. Random forest machine learning algorithms are well-known and described in the art, and typically rely on random forest algorithms that have three main hyperparameters, node size, number of trees, and number of features sampled, that are predetermined. Logistic regression machine learning algorithms also are well known in the art and are used to accomplish binary classification tasks by predicting the probability of an outcome, event, or observation. Those skilled in the art are capable of using random forest machine learning algorithms and/or logistic regression algorithms to develop software or a protocol for carrying out the method of identifying a subject who has, or is at risk of developing a motor neuron disease or other neurological disease using the presence or absence of two more of miRNA selected from miR-151a-3p, miR-199a-5p, miR-150-5p, miR-1290, miR-99a-5p, miR-342-3p, miR-223-3p, miR-652-3p. The software can be provided with one or more kit(s) described herein the first time the purchaser a kit, and loaded onto a computing device integral to, incorporated into, or in communication with a device for conducting qPCR.

Table 21 below provides the miRNA sequences

TABLE 21
miRNA Sequences

| | SEQ | Mirabase | |
|---|---|---|---|
| | ID | (www.mirabase.org) | |
| mi-RNA name | NO: | Accession No. | Sequence |
| 151a-3p | 1 | MIMAT0000757 | CUAGACUGAAGCUCCUUGAGG |
| 151a-5p | 2 | MIMAT0004697 | UCGAGGAGCUCACAGUCUAGU |
| 146a-5p | 3 | MIMAT0000449 | UGAGAACUGAAUUCCAUGGGUU |
| 4454 | 4 | MIMAT0018976 | GGAUCCGAGUCACGGCACCA |
| 10b-5p | 5 | MIMAT0000254 | UACCCUGUAGAACCGAAUUUGUG |
| 199a-3p | 6 | MIMAT0000232 | ACAGUAGUCUGCACAUUGGUUA |
| 199a-5p | 7 | MIMAT0000231 | CCCAGUGUUCAGACUACCUGUUC |
| 29b-3p | 8 | MIMAT0000100 | UAGCACCAUUUGAAAUCAGUGUU |
| 126-5p | 9 | MIMAT0000444 | CAUUAUUACUUUUGGUACGCG |
| 150-5p | 10 | MIMAT0000451 | UCUCCCAACCCUUGUACCAGUG |
| 107 | 11 | MIMAT0000104 | AGCAGCAUUGUACAGGGCUAUCA |
| let-7g-5p | 12 | MIMAT0000414 | UGAGGUAGUAGUUUGUACAGUU |
| 1290 | 13 | MIMAT0005880 | UGGAUUUUUGGAUCAGGGA |
| 95-3p | 14 | MIMAT0000094 | UUCAACGGGUAUUUAUUGAGCA |
| 375-3p | 15 | MIMAT0000728 | UUUGUUCGUUCGGCUCGCGUGA |
| 99a-5p | 16 | MIMAT0000097 | AACCCGUAGAUCCGAUCUUGUG |
| 125b-5p | 17 | MIMAT0000423 | UCCCUGAGACCCUAACUUGUGA |
| 642a-5p | 18 | MIMAT0003312 | GUCCCUCUCCAAAUGUGUCUUG |
| 92a-1-5p | 19 | MIMAT0004507 | AGGUUGGGAUCGGUUGCAAUGCU |
| 362-5p | 20 | MIMAT0000705 | AAUCCUUGGAACCUAGGUGUGAGU |
| 342-3p | 21 | MIMAT0000753 | UCUCACACAGAAAUCGCACCCGU |
| 196a-5p | 22 | MIMAT0000226 | UAGGUAGUUUCAUGUUGUUGGG |
| 155-5p | 23 | MIMAT0000646 | UUAAUGCUAAUCGUGAUAGGGGUU |
| 409-3p | 24 | MIMAT0001639 | GAAUGUUGCUCGGUGAACCCCU |
| 744-5p | 25 | MIMAT0004945 | UGCGGGGCUAGGGCUAACAGCA |
| 223-3p | 26 | MIMAT0000280 | UGUCAGUUUGUCAAAUACCCCA |
| 584-5p | 27 | MIMAT0003249 | UUAUGGUUUGCCUGGGACUGAG |
| 340-5p | 28 | MIMAT0004692 | UUAUAAAGCAAUGAGACUGAUU |
| 421 | 29 | MIMAT0003339 | AUCAACAGACAUUAAUUGGGCGC |
| 486-5p | 30 | MIMAT0002177 | UCCUGUACUGAGCUGCCCCGAG |
| 126-3p | 31 | MIMAT0000445 | UCGUACCGUGAGUAAUAAUGCG |
| 320c | 32 | MIMAT0005793 | AAAAGCUGGGUUGAGAGGGU |
| 320b | 33 | MIMAT0005792 | AAAAGCUGGGUUGAGAGGGCAA |
| let-7d-3p | 34 | MIMAT0004484 | CUAUACGACCUGCUGCCUUUCU |
| 27b-3p | 35 | MIMAT0000419 | UUCACAGUGGCUAAGUUCUGC |
| 148b-3p | 36 | MIMAT0000759 | UCAGUGCAUCACAGAACUUUGU |
| 143-3p | 37 | MIMAT0000435 | UGAGAUGAAGCACUGUAGCUC |
| 326 | 38 | MIMAT0000756 | CCUCUGGGCCCUUCCUCCAG |
| 335-5p | 39 | MIMAT0000765 | UCAAGAGCAAUAACGAAAAAUGU |
| 25-3p | 40 | MIMAT0000081 | CAUUGCACUUGUCUCGGUCUGA |
| 153-3p | 41 | MIMAT0000439 | UUGCAUAGUCACAAAAGUGAUC |
| 19b-3p | 42 | MIMAT0000074 | UGUGCAAAUCCAUGCAAAACUGA |
| let-7c-5p | 43 | MIMAT0000064 | UGAGGUAGUAGGUUGUAUGGUU |
| 652-3p | 44 | MIMAT0003322 | AAUGGCGCCACUAGGGUUGUG |
| 376c-3p | 45 | MIMAT0000720 | AACAUAGAGGAAAUUCCACGU |
| 154-5p | 46 | MIMAT0000452 | UAGGUUAUCCGUGUUGCCUUCG |
| 376a-3p | 47 | MIMAT0000729 | AUCAUAGAGGAAAAUCCACGU |
| 30d-5p | 48 | MIMAT0000245 | UGUAAACAUCCCCGACUGGAAG |
| 381-3p | 49 | MIMAT0000736 | UAUACAAGGGCAAGCUCUCUGU |
| 142-3p | 50 | MIMAT0000434 | UGUAGUGUUUCCUACUUUAUGGA |
| 451a | 51 | MIMAT0001631 | AAACCGUUACCAUUACUGAGUU |
| 23a-3p | 52 | MIMAT0000078 | AUCACAUUGCCAGGGAUUUCC |
| 103a-3p | 53 | MIMAT0000101 | AGCAGCAUUGUACAGGGCUAUGA |
| 30c-5p | 54 | MIMAT0000244 | UGUAAACAUCCUACACUCUCAGC |

Any or all “T” nucleotide bases in the sequences of the miRNAs shown in Table 14 above may be substituted with a “U” nucleotide base.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The embodiments are generally disclosed herein using affirmative language to describe the numerous features and aspects thereof. The embodiments also specifically include features in which particular subject matter is excluded, in full or in part, such as substances or materials, method steps and conditions, protocols, or procedures. For example, in certain embodiments or aspects of the invention, materials and/or method steps are excluded. Thus, even though the embodiments are generally not expressed herein in terms of what they do not include, aspects that are not expressly excluded are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. A composition “consisting essentially of” refers to a composition that includes only the active ingredients claimed (e.g., active ingredient (AI) or active pharmaceutical ingredient (API); e.g., L-serine, a salt, metabolic precursor, derivative or conjugate thereof); which composition may include other ingredients such as formulation materials, excipients, additives, carriers, preservatives, diluents, solvents, fillers, salts, buffers, coatings, binders, and lubricating agents; and which composition excludes other APIs not claimed.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

What is claimed is:

1. A method of identifying a subject who has, or is at risk of developing a motor neuron disease and/or neurodegenerative disorder selected from the group consisting of three or more of amyotrophic lateral sclerosis (ALS), primary lateral sclerosis (PLS), Alzheimer's Disease (AD), Mild Cognitive Impairment (MCI), Parkinson's Disease (PD), Multiple Sclerosis (MS), Huntington's Disease (HD), prion diseases such as Creutzfeldt-Jakob disease (CJD), Lewy Body Disease (LBD), Multiple System Atrophy (MSA), and Frontotemporal Dementia (FTD), Progressive Supranuclear Palsy (PSP), and controls without neurological symptoms the method comprising:
(a) providing a final miRNA panel comprising a list of miRNAs for each of the motor neuron diseases and/or neurodegenerative disorders, wherein each miRNA in each list has a coefficient associated therewith in which the coefficient represents the relative contribution each respective miRNA has in identifying a subject as having or not having the motor neuron disease and/or neurodegenerative disorder related to that miRNA, the list capable of identifying a subject who has, or is at risk of developing the respective motor neuron disease and/or neurodegenerative disorder;
(b) determining a presence or amount of two or more micro-RNAs (miRNAs) in a subject's circulating blood, wherein the two or more miRNA are selected from the final miRNA panel, without determining a presence or amount of the miRNAs from neural-derived exosomes;
(c) determining the Cq and delta Cq values, and coefficients for each of the two or more miRNAs in the subject's circulating blood; and
(c) determining if the subject has, or is at risk of developing a motor neuron disease and/or neurodegenerative disorder selected from the group consisting of three or more of ALS, PLS, AD, MCI, PD, MS, HD, CJD, LBD, MSA, FTD, and PSP according to the coefficients of the two or more miRNAs in the sample.

2. The method of claim 1, wherein the method further comprises a method of preventing or treating a motor neuron disease and/or neurodegenerative disorder selected from the group consisting of ALS, PLS, AD, MCI, PD, MS, HD, CJD, LBD, MSA, FTD, and PSP in a subject who has, or is at risk of developing ALS, PLS, AD, MCI, PD, MS, HD, CJD, LBD, MSA, FTD, and PSP the method comprising:
(c) administering a therapeutically effective amount of an ALS, PLS, AD, MCI, PD, MS, HD, CJD, LBD, MSA, FTD, and PSP drug to the subject when the determining of (c) determines that the subject has, or is at risk of developing ALS, PLS, AD, MCI, PD, MS, HD, CJD, LBD, MSA, FTD, and PSP.

3. The method of claim 1, wherein the amount of the micro RNAs determined in (b) is at least 1.0-fold higher or lower than a baseline amount, thereby indicating the subject has, or is a risk of developing a motor neuron disease and/or neurodegenerative disorder selected from the group consisting of at least ALS, PLS, AD, MCI, PD, MS, HD, CJD, LBD, MSA, FTD, and PSP.

4. The method of claim 1, wherein the subject is a human.

5. The method of claim 3, wherein the baseline amount is an average, mean or absolute amount of any one of the miRNAs present in a healthy control subject.

6. The method of claim 1, further comprising monitoring the progression of the motor neuron disease and/or neurodegenerative disorder in the subject, wherein the method is conducted two or more times for the subject.

7. A method of identifying a subject who has, or is at risk of developing a motor neuron disease and/or neurodegenerative disorder comprising,
(a) determining a presence or amount of two or more micro-RNAs (miRNAs) in a sample obtained from the subject wherein the two or more miRNA are selected from the group consisting of miR-151a-3p, miR-199a-5p, miR-150-5p, miR-1290, miR-99a-5p, miR-342-3p, miR-223-3p, miR-652-3p, miR-335-5p, miR-let-7g-5p, miR-let-7c-5p, miR-486-5p, miR-155-5p, miR-4454, miR-10b-5p, miR-126-5p, 362-5p, miR-146a-5p, miR-125b-5p, and miR-199a-3p;
(b) determining the Cq and delta Cq values, and coefficients for each of the two or more miRNAs, the coefficients representing the relative contribution of each miRNA to the risk determination; and
(c) determining if the subject has, or is at risk of developing a motor neuron disease and/or neurodegenerative disorder according to the coefficients of the three or more miRNAs in the sample.
